(19) United States
(12) Patent Application Publication
(10) Pub. No.: US 2026/0355353 A1
(43) Pub. Date: Nov. 12, 2026

(54) Interleukin-2 and Interleukin-12 for Cancer Therapy

(71) Applicant: KRYSTAL BIOTECH, INC., Pittsburgh, PA (US)

(72) Inventors: Suma Krishnan, Pittsburgh, PA (US); Trevor Parry, San Diego, CA (US)

(21) Appl. No.: 19/161,428

(22) PCT Filed: Mar. 1, 2024
(86) PCT No.: PCT/US2024/018084

§ 371 Date: Sep. 2, 2025

Related U.S. Application Data
(60) Provisional application No. 63/449,533, filed on Mar. 2, 2023.

(51) Int. Cl. A61K 48/00; A61K 38/20; A61P 35/00

(52) U.S. Cl. CPC A61K 48/005; A61K 38/2013; A61K 38/208; A61K 48/0033; A61K 48/0041; A61K 48/0075; A61P 35/00

(57) ABSTRACT

The present disclosure provides a first polynucleotide (e.g., a recombinant or isolated first polynucleotide) encoding an Interleukin (IL)-2 polypeptide and a second polynucleotide (e.g., a recombinant or isolated second polynucleotide) encoding an IL-12 polypeptide; recombinant nucleic acids and/or gene delivery vehicles comprising the same; compositions, formulations, and medicaments comprising the one or more of the polynucleotides, recombinant nucleic acids, and/or gene delivery vehicles; methods of their use (e.g., for the treatment of cancer); and articles of manufacture or kits thereof.

### CROSS REFERENCE TO RELATED APPLICATION

This application claims the priority benefit of U.S. Provisional Application Ser. No. 63/449,533, filed Mar. 2, 2023, which is incorporated herein by reference in its entirety.

### SUBMISSION OF SEQUENCE LISTING ON ASCII TEXT FILE

The Sequence Listing associated with this application is filed in electronic format via Patent Center and is hereby incorporated by reference into the specification in its entirety. The name of the file containing the Sequence Listing is 2401084.xml. The size of the file is 70,610 bytes, and the file was created on Mar. 1, 2024.

### FIELD OF THE INVENTION

The present disclosure relates, in part, to a first recombinant polynucleotide encoding an Interleukin (IL)-2 polypeptide and a second recombinant polynucleotide encoding an IL-12 polypeptide, recombinant nucleic acids and/or gene delivery vehicles comprising the same, pharmaceutical compositions and formulations thereof, and methods of their use (e.g., for treating cancer).

### BACKGROUND

Cancer is among the leading causes of death worldwide. Despite significant advances in clinical care and treatment methods, more effective cancer treatment options are still needed to prolong survival and decrease cancer death rates.

All references cited herein, including patent applications, patent publications, non-patent literature, and NCBI/UniProtKB/Swiss-Prot Accession numbers are herein incorporated by reference in their entirety, as if each individual reference were specifically and individually indicated to be incorporated by reference.

### BRIEF SUMMARY

In order to meet these and other needs, provided herein are polynucleotides (e.g., recombinant and/or isolated polynucleotides), in particular a first polynucleotide encoding an Interleukin (IL) 2 polypeptide and a second polynucleotide encoding an IL-12 polypeptide, for use in recombinant nucleic acids and/or gene delivery vehicles (e.g., a viral vector and/or a non-viral vector such as a lipid nanoparticle), pharmaceutical compositions and/or formulations comprising the polynucleotides, recombinant nucleic acids, and/or gene delivery vehicles, medicaments comprising the polynucleotides, recombinant nucleic acids, gene delivery vehicles, and/or pharmaceutical compositions or formulations, and/or methods of their use e.g., for treating cancer in a subject in need thereof.

Accordingly, certain aspects of the present disclosure relate to a composition or a pharmaceutical composition comprising a first recombinant polynucleotide encoding an IL-2 polypeptide and a second recombinant polynucleotide encoding an IL-12 polypeptide. In some embodiments, the first polynucleotide and the second polynucleotide are contiguous. In some embodiments, the first polynucleotide and the second polynucleotide are non-contiguous. In some embodiments, the first polynucleotide and/or the second polynucleotide comprises deoxyribonucleic acid (DNA). In some embodiments, the DNA is linear DNA or circular DNA. In some embodiments, the first polynucleotide and/or the second polynucleotide comprises ribonucleic acid (RNA). In some embodiments, the RNA is self-replicating. In some embodiments, the RNA is self-amplifying. In some embodiments, the RNA is not self-replicating. In some embodiments, the RNA is not self-amplifying. In some embodiments, the RNA is a messenger RNA (mRNA) or a modified mRNA (mmRNA). In some embodiments, the mRNA and/or the mmRNA further comprises a 5′ untranslated region (UTR), a 3′ UTR, a polyadenylation (poly(A)) tail, and/or a 5′ cap analog.

In some embodiments, the pharmaceutical composition comprises recombinant nucleic acids comprising any one or more of the recombinant polynucleotides described herein. In some embodiments, the recombinant nucleic acid is a linear DNA, a circular DNA, an RNA, a mRNA, and/or a modified mRNA. In some embodiments, the recombinant nucleic acid is a recombinant viral genome.

In some embodiments that may be combined with any of the preceding embodiments, the pharmaceutical composition comprises one or more gene delivery vehicles comprising the first polynucleotide and/or the second polynucleotide. In some embodiments, the one or more gene delivery vehicles are one or more of a viral gene delivery vehicle and/or a non-viral gene delivery vehicle. In some embodiments, the one or more gene delivery vehicles are one or more of a viral vector and/or a non-viral vector.

In some embodiments, the one or more gene delivery vehicles are one or more viral vectors. In some embodiments, the viral vector is pseudotyped. In some embodiments, the viral vector comprises a recombinant viral genome. In some embodiments, the recombinant viral genome comprises the first polynucleotide and/or the second polynucleotide. In some embodiments, the recombinant viral genome is replication competent. In some embodiments, the recombinant viral genome is replication defective. In some embodiments, the recombinant viral genome is a recombinant oncolytic virus genome. In some embodiments, the recombinant viral genome is not a recombinant oncolytic virus genome. In some embodiments, the recombinant viral genome is selected from a recombinant adenovirus genome, a recombinant retrovirus genome, a recombinant adeno-associated virus (AAV) genome, a recombinant herpes virus genome, a recombinant poxvirus genome, a recombinant bacteriophage genome, a recombinant alphavirus genome, a recombinant picornavirus genome, a recombinant iridovirus genome, a recombinant Newcastle disease virus genome, a recombinant baculovirus genome, a recombinant geminivirus genome, a recombinant caulimovirus genome, and any combinations and/or derivatives thereof.

In some embodiments, the one or more gene delivery vehicles are one or more viral vectors. In some embodiments, the viral vector is pseudotyped. In some embodiments, the viral vector comprises the first polynucleotide and/or the second polynucleotide. In some embodiments, the viral vector is replication competent. In some embodiments, the viral vector is replication defective. In some embodiments, the viral vector is an oncolytic virus. In some embodiments, the viral vector is not an oncolytic virus. In some embodiments, the viral vector is selected from an adenovirus, a retrovirus, an AAV, a herpes virus, a poxvirus, a bacteriophage, an alphavirus, a picornavirus, a iridovirus, a Newcastle disease virus, a baculovirus, a geminivirus, a caulimovirus, and any combinations and/or derivatives thereof.

In some embodiments, the one or more gene delivery vehicles are one or more non-viral vectors. In some embodiments, the non-viral vector is a chemical compound, a bacterium, a mammalian cell, or a physical delivery system. In some embodiments, the chemical compound is selected from a polymer compound, a lipid compound, an inorganic compound, and any combinations and/or derivatives thereof. In some embodiments, the polymer compound is a natural polymer, a synthetic polymer, a biopolymer, a biodegradable polymer, a cationic polymer, a protein polymer, a polysaccharide polymer, or any combinations and/or derivatives thereof. In some embodiments, the lipid compound is selected from a nanoparticle, a lipid nanoparticle, a liposome, a cationic liposome, a solid lipid nanoparticle, a lipid emulsion, a lipidoid, a cytofectin, a lipid emulsion, a surfactant, a gemini surfactant, and any combinations and/or derivatives thereof. In some embodiments, the inorganic compound is selected from a nanocarrier, a DNA nanclew, a gold nanoparticle, a carbon nanotube, a graphene, a quantum dot, an up-conversion nanoparticle, a silica nanoparticle, an iron oxide, a ferritin, and any combinations and/or derivatives thereof. In some embodiments, the physical delivery system is selected from electroporation, gene gun, jet gun, ultrasound, nucleofection, hydrodynamic gene delivery, needle injection, microinjection, ballistic DNA injection, sonoporation, photoporation, laser pulse, magnetofection, magnetoporation, magnetic particles, hydroporation, and any combinations and/or derivatives thereof.

Other aspects of the present disclosure relate to the use of any of the recombinant polynucleotides, recombinant nucleic acids and/or gene delivery vehicles comprising the recombinant polynucleotides, and/or pharmaceutical compositions described herein as a medicament.

Other aspects of the present disclosure relate to the use of any of the recombinant polynucleotides, recombinant nucleic acids and/or gene delivery vehicles comprising the recombinant polynucleotides, and/or pharmaceutical compositions described herein in a therapy.

Other aspects of the present disclosure relate to the use of any of the recombinant polynucleotides, recombinant nucleic acids and/or gene delivery vehicles comprising the recombinant polynucleotides, and/or pharmaceutical compositions described herein in the preparation of a medicament for treating cancer.

Other aspects of the present disclosure relate to a method of expressing, enhancing, increasing, augmenting, and/or supplementing the levels of an IL-2 and/or IL-12 polypeptide in one or more cells of a subject comprising administering to the subject an effective amount of any of the recombinant polynucleotides, recombinant nucleic acids and/or gene delivery vehicles comprising the recombinant polynucleotides, pharmaceutical compositions, and/or medicaments described herein. In some embodiments, the one or more cells are one or more cells of the respiratory tract, airway epithelial, and/or lung. In some embodiments that may be combined with any of the preceding embodiments, the subject is a human. In some embodiments that may be combined with any of the preceding embodiments, the recombinant polynucleotides, recombinant nucleic acids and/or gene delivery vehicles comprising the recombinant polynucleotides, pharmaceutical compositions, and/or medicaments are administered topically, transdermally, subcutaneously, epicutaneously, intradermally, orally, sublingually, buccally, rectally, vaginally, intravenously, intraarterially, intramuscularly, intraosseously, intracardially, intraperitoneally, transmucosally, intravitreally, subretinally, suprachoroidally, intracranially, intrathecally, intraventricularly, intraarticularly, peri-articularly, intratumorally, locally, or via inhalation to the subject. In some embodiments that may be combined with any of the preceding embodiments, the recombinant polynucleotides, recombinant nucleic acids and/or gene delivery vehicles comprising the recombinant polynucleotides, pharmaceutical compositions, and/or medicaments are administered orally, intranasally, intratracheally, or via inhalation to the subject. In some embodiments, the recombinant polynucleotides, recombinant nucleic acids and/or gene delivery vehicles comprising the recombinant polynucleotides, pharmaceutical compositions, and/or medicaments are administered intranasally or via inhalation to the subject. In some embodiments, the recombinant polynucleotides, recombinant nucleic acids and/or gene delivery vehicles comprising the recombinant polynucleotides, pharmaceutical compositions, and/or medicaments are administered via inhalation to the subject. In some embodiments, the recombinant polynucleotides, recombinant nucleic acids and/or gene delivery vehicles comprising the recombinant polynucleotides, pharmaceutical compositions, and/or medicaments are administered using a dry powder inhaler, a pressurized metered dose inhaler, a soft mist inhaler, a nebulizer, or an electrohydrodynamic aerosol device. In some embodiments, the recombinant polynucleotides, recombinant nucleic acids and/or gene delivery vehicles comprising the recombinant polynucleotides, pharmaceutical compositions, and/or medicaments are administered using a nebulizer. In some embodiments, the nebulizer is a vibrating mesh nebulizer.

Other aspects of the present disclosure relate to a method of providing prophylactic, palliative, or therapeutic relief of one or more signs or symptoms of cancer in a subject in need thereof comprising administering to the subject an effective amount of any of the recombinant polynucleotides, recombinant nucleic acids and/or gene delivery vehicles comprising the recombinant polynucleotides, pharmaceutical compositions, and/or medicaments described herein. In some embodiments, the cancer is selected from a solid tumor, a hematologic cancer, bladder cancer, brain cancer, breast cancer, colon cancer, gastric cancer, glioma, head cancer, leukemia, liver cancer, lung cancer, lymphoma, myeloma, neck cancer, ovarian cancer, melanoma, pancreatic cancer, renal cancer, salivary cancer, skin cancer, stomach cancer, thymic epithelial cancer, and thyroid cancer. In some embodiments, the cancer is small-cell lung cancer, non-small cell lung cancer, adenocarcinoma of the lung, or squamous carcinoma of the lung. In some embodiments, the cancer is non-small cell lung cancer. In some embodiments, the cancer is osteosarcoma. In some embodiments that may be combined with any of the preceding embodiments, the subject is a human. In some embodiments that may be combined with any of the preceding embodiments, the recombinant polynucleotides, recombinant nucleic acids and/or gene delivery vehicles comprising the recombinant polynucleotides, pharmaceutical compositions, and/or medicaments are administered topically, transdermally, subcutaneously, epicutaneously, intradermally, orally, sublingually, buccally, rectally, vaginally, intravenously, intraarterially, intramuscularly, intraosseously, intracardially, intraperitoneally, transmucosally, intravitreally, subretinally, suprachoroidally, intracranially, intrathecally, intraventricularly, intraarticularly, peri-articularly, intratumorally, locally, or via inhalation to the subject. In some embodiments that may be combined with any of the preceding embodiments, the recombinant polynucleotides, recombinant nucleic acids and/or gene delivery vehicles comprising the recombinant polynucleotides, pharmaceutical compositions, and/or medicaments are administered orally, intranasally, intratracheally, or via inhalation to the subject. In some embodiments that may be combined with any of the preceding embodiments, the recombinant polynucleotides, recombinant nucleic acids and/or gene delivery vehicles comprising the recombinant polynucleotides, pharmaceutical compositions, and/or medicaments are administered orally, intranasally, intratracheally, or via inhalation to the subject. In some embodiments, the recombinant polynucleotides, recombinant nucleic acids and/or gene delivery vehicles comprising the recombinant polynucleotides, pharmaceutical compositions, and/or medicaments are administered intranasally or via inhalation to the subject. In some embodiments, the recombinant polynucleotides, recombinant nucleic acids and/or gene delivery vehicles comprising the recombinant polynucleotides, pharmaceutical compositions, and/or medicaments are administered via inhalation to the subject. In some embodiments, the recombinant polynucleotides, recombinant nucleic acids and/or gene delivery vehicles comprising the recombinant polynucleotides, pharmaceutical compositions, and/or medicaments are administered using a dry powder inhaler, a pressurized metered dose inhaler, a soft mist inhaler, a nebulizer, or an electrohydrodynamic aerosol device. In some embodiments, the recombinant polynucleotides, recombinant nucleic acids and/or gene delivery vehicles comprising the recombinant polynucleotides, pharmaceutical compositions, and/or medicaments are administered using a nebulizer. In some embodiments, the nebulizer is a vibrating mesh nebulizer.

Other aspects of the present disclosure relate to a method of treating cancer in a subject in need thereof comprising administering to the subject an effective amount of any of the recombinant polynucleotides, recombinant nucleic acids and/or gene delivery vehicles comprising the recombinant polynucleotides, pharmaceutical compositions, and/or medicaments described herein. In some embodiments, the cancer is selected from carcinoma, lymphoma, blastoma, sarcoma, a neuroendocrine tumor, mesothelioma, schwannoma, meningioma, adenocarcinoma, melanoma, leukemia, and lymphoid malignancy. In some embodiments, the cancer is selected from a solid tumor, a hematologic cancer, bladder cancer, brain cancer, breast cancer, colon cancer, gastric cancer, glioma, head cancer, leukemia, liver cancer, lung cancer, lymphoma, myeloma, neck cancer, ovarian cancer, melanoma, pancreatic cancer, renal cancer, salivary cancer, skin cancer, stomach cancer, thymic epithelial cancer, and thyroid cancer. In some embodiments, the cancer is small-cell lung cancer, non-small cell lung cancer, adenocarcinoma of the lung, or squamous carcinoma of the lung. In some embodiments, the cancer is non-small cell lung cancer. In some embodiments, the cancer is osteosarcoma. In some embodiments that may be combined with any of the preceding embodiments, the subject is a human. In some embodiments that may be combined with any of the preceding embodiments, the recombinant polynucleotides, recombinant nucleic acids and/or gene delivery vehicles comprising the recombinant polynucleotides, pharmaceutical compositions, and/or medicaments are administered topically, transdermally, subcutaneously, epicutaneously, intradermally, orally, sublingually, buccally, rectally, vaginally, intravenously, intraarterially, intramuscularly, intraosseously, intracardially, intraperitoneally, transmucosally, intravitreally, subretinally, suprachoroidally, intracranially, intrathecally, intraventricularly, intraarticularly, peri-articularly, intratumorally, locally, or via inhalation to the subject. In some embodiments that may be combined with any of the preceding embodiments, the recombinant polynucleotides, recombinant nucleic acids and/or gene delivery vehicles comprising the recombinant polynucleotides, pharmaceutical compositions, and/or medicaments are administered orally, intranasally, intratracheally, or via inhalation to the subject. In some embodiments, the recombinant polynucleotides, recombinant nucleic acids and/or gene delivery vehicles comprising the recombinant polynucleotides, pharmaceutical compositions, and/or medicaments are administered intranasally or via inhalation to the subject. In some embodiments, the recombinant polynucleotides, recombinant nucleic acids and/or gene delivery vehicles comprising the recombinant polynucleotides, pharmaceutical compositions, and/or medicaments are administered via inhalation to the subject. In some embodiments, the recombinant polynucleotides, recombinant nucleic acids and/or gene delivery vehicles comprising the recombinant polynucleotides, pharmaceutical compositions, and/or medicaments are administered using a dry powder inhaler, a pressurized metered dose inhaler, a soft mist inhaler, a nebulizer, or an electrohydrodynamic aerosol device. In some embodiments, the recombinant polynucleotides, recombinant nucleic acids and/or gene delivery vehicles comprising the recombinant polynucleotides, pharmaceutical compositions, and/or medicaments are administered using a nebulizer. In some embodiments, the nebulizer is a vibrating mesh nebulizer.

Other aspects of the present disclosure relate to an article of manufacture or kit comprising any of the recombinant polynucleotides, recombinant nucleic acids and/or gene delivery vehicles comprising the recombinant polynucleotides, pharmaceutical compositions, and/or medicaments described herein and instructions for administration thereof.

### BRIEF DESCRIPTION OF THE DRAWINGS

FIGS. 1A-1B depict secreted cytokine levels (IL-12, FIG. 1A; IL-2, FIG. 1B) by HEK293FT cells at 24-hours post infection with a modified herpes simplex virus encoding human IL-12 (FIG. 1A) and IL-2 (FIG. 1B) transgene at a multiplicity of infection (MOI) of 1.

FIGS. 2A-2B depict in vitro bioactivity assay(s) of HSV-derived IL-12 (FIG. 2A) and IL-2 (FIG. 2B) compared to commercially available recombinant proteins as a function of IFNγ release from splenocytes (FIG. 2A) or HEK-Blue™ IL2 reporter cells (FIG. 2B). Data indicative of cells assayed in triplicate, and data are presented as means±standard deviation (SD).

FIGS. 3A-3H show the in vivo pharmacokinetic evaluation of HSV-IL12/HSV-IL2 following intradermal administration in healthy mice. FIG. 3A and FIG. 3C depict genome (circles) and transcript (square) levels in skin of C57BL/6 mice post-intradermal administration of HSV-IL12/HSV-IL2 at the indicated time points. qPCR (FIG. 3A circles and FIG. 3C circles) and qRT-PCT (FIG. 3A squares and FIG. 3C squares) were performed to measure IL12 (FIG. 3A) and IL2 (FIG. 3C) genomes and transcripts, respectively. Data are indicative of samples run in duplicate and displayed as mean±standard error of the mean (SEM) of n=2-3 animals per group. FIG. 3B and FIG. 3D depict IL-12 (FIG. 3B) and IL-2 (FIG. 3D) protein concentrations of skin homogenates following HSV-IL12/HSV-IL2 intradermal administration at the indicated time points. Skin tissue was homogenized and assayed by ELISA in duplicate. Protein concentrations of homogenates were determined by BCA assay, and IL-12 or IL-2 concentrations were normalized to total protein. Data are displayed as mean±SEM of n=2-3 animals per group. FIG. 3E and FIG. 3F depict IL-12 (FIG. 3E) and IL-2 (FIG. 3F) protein concentrations in serum following HSV-IL12/HSV-IL2 intradermal (ID) administration or recombinant protein intravenous (IV) administration at the indicated time points. Protein concentrations of serum samples were determined by ELISA. Values indicate the difference in magnitude between peak exposure (5 minutes for IV recombinant protein vs. 24-hours for ID vector administration). Data are displayed as means±SEM with symbols indicating independent animals (n=2-3 per group). Statistical significance was determined by a one-way ANOVA with Tukey's post-hoc analysis. ***=p<0.001. FIG. 3G and FIG. 3H depict IL-12 (FIG. 3G) and IL-2 (FIG. 3H) protein concentrations in skin biopsies following HSV-IL12/HSV-IL2 ID administration or recombinant protein IV administration at the indicated time points. Cytokine concentrations were normalized to total protein. Data are displayed as means±SEM with symbols indicating independent animals (n=2-3 per group). Statistical significance was determined by a one-way ANOVA with Tukey's post-hoc analysis. **=p<0.01; ****=p<0.0001.

FIGS. 4A-4/show the in vivo evaluation of HSV-IL12/HSV-IL2 following intratracheal administration in healthy mice. FIGS. 4A-4E depict the in vivo evaluation of HSV-IL12/HSV-IL2 in BALB/c female mice following intratracheal administration with either vehicle control or HSV-IL12/HSV-IL2 high or mid dose (~10⁸ or 10⁷ total PFU, respectively) on days 0 and 7. Animals were sacrificed on day 8, 24-hours following their last dose. FIG. 4A depicts once weekly body weights in BALB/c female mice. Data are displayed as means±SEM. FIGS. 4B-4E depict bronchoalveolar lavage fluid (BALF) (FIGS. 4B-4C) and whole lungs (FIGS. 4D-4E) that were collected postmortem and analyzed for IL-12 and IL-2 concentrations by ELISA. Whole lung homogenate cytokine concentrations were normalized to total protein. Data are displayed as means±SEM, with symbols representing independent animals. n=2-3 per group. For FIGS. 4B-4E, statistical significance was determined using a one-way ANOVA with Tukey's post-hoc analysis. * p=<0.05; **=p<0.01. FIGS. 4F-4/depict the in vivo evaluation in healthy mice following the intratracheal administration of HSV-IL12/HSV-IL2 (~10⁷ total PFU) or the intravenous administration of IL-12 and IL-2 recombinant proteins (125 ng and 600 ng, respectively) at murine equivalent doses that demonstrated clinical efficacy and toxicity in humans. Untreated animals served as negative controls. FIGS. 4F-4G depict serum that was sampled at 5 minutes and 24-hours post-administration to compare peak systemic cytokine exposure for recombinant protein and vector treatment, respectively, and assayed by ELISA. Values indicate the difference in magnitude between peak exposures. FIGS. 4H-4/depict whole lungs that were taken at indicated time points to measure IL-12 and IL-2 concentrations. Cytokine concentrations were normalized to total protein. For FIGS. 4F-4I, data are displayed as means±SEM with symbols indicating independent animals (n=2-3 per group), and statistical significance was determined by a one-way ANOVA with Tukey's post-hoc analysis. *=p<0.05, **=p<0.01, ***=p<0.001.

FIGS. 5A-5S depict HSV-IL12/HSV-IL2 efficacy in an in vivo murine model of melanoma. FIGS. 5A-5B depict combinatorial HSV-IL12/HSV-IL2 administered via intratumoral injection enhanced survival in B16F10 melanoma-bearing mice compared to control or single vector treatment. FIG. 5A shows the study design. Euthanasia criteria were either tumor area ≥150 mm² or body weight loss ≥20% of their pre-study body weight. Vectors were administered at ~10⁸ total PFU. SC: subcutaneous. FIG. 5B depicts survival curves for vehicle control versus HSV-IL12 alone, HSV-IL2 alone, HSV-GMCSF alone, and combinatorial HSV-IL12/HSV-IL2 dosed mice. Survival data are displayed as individual animals and were analyzed using a Log-Rank test corrected for multiple comparisons to compare each treatment group to vehicle control. ns=not significant. **=p<0.01. FIGS. 5C-5E depict weekly HSV-IL12/HSV-IL2 intratumoral injection with additional maintenance dose improved survival of B16F10 melanoma-bearing mice. FIG. 5C shows the study design. Euthanasia criteria were either tumor area ≥150 mm² or body weight loss ≥20% of their pre-study body weight. Vectors were administered at ~10⁸ total PFU. SC: subcutaneous. FIG. 5D depicts tumor measurements for each treatment group. Data are displayed as means±SEM of n=10 animals per group and were analyzed using a Mixed-effects analysis. FIG. 5E shows survival data displayed as individual animals and were analyzed using a Log-Rank test. ***=p<0.001. FIGS. 5F-5/depict HSV-IL12/HSV-IL2 treatment of primary B16F10 melanomas resulted in control of rechallenge tumors. FIG. 5F shows the study design. Euthanasia criteria were either tumor area ≥150 mm² or body weight loss ≥20% of their pre-study body weight. Vectors were administered at ~10⁸ total PFU. SC: subcutaneous. FIGS. 5G-5H depict tumor measurements for each treatment group during the initial phase (primary tumor; FIG. 5G) and rechallenge phase (rechallenge tumor; FIG. 5H). Data are displayed as means±SEM of n=4-5 animals per group. For the rechallenge phase, 5 naïve age-matched C57BL/6 animals were inoculated with tumors to serve as positive controls for tumor growth. FIG. 5/shows survival data displayed as individual animals. Statistical significance was determined using a Mixed-effects model (FIGS. 5G-5H) or a Log-rank test (FIG. 5/). *=p<0.05; **=p<0.01. FIGS. 5J-5S depict HSV-IL12/HSV-IL2 treatment of a primary B16F10 melanoma resulted in an abscopal effect against a secondary B16F10 tumor. FIG. 5J shows a schematic of tumor inoculation and the study design. Euthanasia criteria were either tumor area ≥150 mm² or body weight loss ≥20% of their pre-study body weight. Vectors were administered at ~10⁸ total PFU. FIGS. 5K-5L depict tumor measurements of the primary/treated tumor (FIG. 5K) and secondary tumor (FIG. 5L) in the day 0 secondary tumor inoculation group. Data are presented as means±SEM of n=5 animals per group. FIG. 5M depicts survival data in the day 0 secondary tumor inoculation group. Data are displayed as individual animals. FIGS. 5N-5O depict tumor measurements of the primary/treated tumor (FIG. 5N) and secondary tumor (FIG. 5O) in the day 4 secondary tumor inoculation group. Data are presented as means±SEM of n=5 animals per group. FIG. 5P depicts survival data in the day 4 secondary tumor inoculation group. Data are displayed as individual animals. FIGS. 5Q-5R depict tumor measurements of the primary/treated tumor (FIG. 5Q) and secondary tumor (FIG. 5R) in the day 10 secondary tumor inoculation group. Data are presented as means±SEM of n=5 animals per group. FIG. 5S depicts survival data in the day 10 secondary tumor inoculation group. Data are displayed as individual animals. Statistical significance was determined using a Mixed-effects analysis (tumor area) or a Log-Rank test (survival). *=p<0.05; **=p<0.01; ****=p<0.0001.

FIGS. 6A-6H depict HSV-IL12/HSV-IL2 efficacy in an in vivo murine model of osteosarcoma (e.g. osteosarcoma lung metastases). FIGS. 6A-6D show combinatorial HSV-IL12/HSV-IL2 administered intratracheally enhanced tumor regression and survival in a K7M2 osteosarcoma lung metastasis model compared to control or single vector treatment. FIG. 6A depicts the study design. Euthanasia was performed if body weight loss was ≥20% of their pre-study body weight. All vectors were administered at ~10⁷ total PFU. IV: intravenous. FIGS. 6B-6C show body weight (FIG. 6B) and survival (FIG. 6C) data. Body weight data are displayed as means±SEM with n=5 animals per group. Survival data are displayed as individual animals, and statistical significance was determined using a Log-Rank-test correcting for multiple comparisons. FIG. 6D depicts representative H&E stained lung sections from animals that survived to day 100. FIGS. 6E-6H show intratracheal administration of HSV-IL12/HSV-IL2 minimizes initial K7M2 lung tumor outgrowth and delays tumor recurrence without additional therapeutic intervention. FIG. 6E depicts a schematic of the study design. Euthanasia was performed if body weight loss was ≥20% of their pre-study body weight. All vectors were administered at ~10⁷ total PFU. For the rechallenge phase, 5 untreated age-matched BALB/c animals were inoculated with tumors to serve as positive controls for tumor growth. IV: intravenous. FIGS. 6F-6G show body weight measurements of animals during primary (FIG. 6F) and rechallenge (FIG. 6G) phases. Data are displayed as means±SEM of n=4-5 animals per group. FIG. 6H depicts survival data displayed as individual animals. Statistical significance was determined using a Log-Rank test. **=p<0.01; ***=p<0.001.

### DETAILED DESCRIPTION

The following description sets forth exemplary methods, parameters, and the like. It should be recognized, however, that such a description is not intended as a limitation on the scope of the present disclosure but is instead provided as a description of exemplary embodiments.

### I. GENERAL TECHNIQUES

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Gene Transfer Vectors for Mammalian Cells (J. M. Miller and M. P. Calos, eds., 1987); PCR: The Polymerase Chain Reaction, (Mullis et al., eds., 1994); Short Protocols in Molecular Biology (Wiley and Sons, 1999).

### II. DEFINITIONS

Before describing the present disclosure in detail, it is to be understood that the present disclosure is not limited to particular compositions or biological systems, which can, of course, vary. It is also to be understood that the terminology used herein is for the purpose of describing particular embodiments only and is not intended to be limiting.

As used herein, the singular forms “a”, “an”, and “the” include plural referents unless the content clearly dictates otherwise. Thus, for example, reference to “a molecule” optionally includes a combination of two or more such molecules, and the like.

As used herein, the term “and/or” may include any and all combinations of one or more of the associated listed items. For example, the term “a and/or b” may refer to “a alone”, “b alone”, “a or b”, or “a and b”; the term “a, b, and/or c” may refer to “a alone”, “b alone”, “c alone”, “a or b”, “a or c”, “b or c”, “a, b, or c”, “a and b”, “a and c”, “b and c”, or “a, b, and c”; etc.

As used herein, the term “about” refers to the usual error range for the respective value readily known to the skilled person in this technical field. Reference to “about” a value or parameter herein includes (and describes) embodiments that are directed to that value or parameter per se.

It is understood that aspects and embodiments of the present disclosure include “comprising”, “consisting”, and “consisting essentially of” aspects and embodiments.

As used herein, the terms “polynucleotide”, “nucleic acid sequence”, “nucleic acid”, and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog, and inter-nucleotide modifications.

As used herein, a nucleic acid is “operatively linked” or “operably linked” when it is placed into a functional relationship with another nucleic acid sequence. For example, a promoter or enhancer is operably linked to a coding sequence if it affects the transcription of the sequence, or a ribosome binding site is operably linked to a coding sequence if it is positioned so as to facilitate translation. Generally, “operatively linked” or “operably linked” means that the DNA or RNA sequences being linked are contiguous.

As used herein, an “open reading frame” or “ORF” refers to a continuous stretch of nucleic acids, either DNA or RNA, that encode a protein or polypeptide. Typically, the nucleic acids comprise a translation start signal or initiation codon, such as ATG or AUG, and a termination codon.

As used herein, an “untranslated region” or “UTR” refers to untranslated nucleic acids at the 5′ and/or 3′ ends of an open reading frame. The inclusion of one or more UTRs in a polynucleotide may affect post-transcriptional regulation, mRNA stability, translation of the polynucleotide, etc.

As used herein, the terms “polypeptide,” “protein,” and “peptide” are used interchangeably and may refer to a polymer of two or more amino acids.

As used herein, a “subject”, “host”, or an “individual” refers to any animal classified as a mammal, including humans, domestic and farm animals, and zoo, sports, or pet animals, such as dogs, horses, cats, cows, as well as animals used in research, such as mice, rats, hamsters, rabbits, and non-human primates, etc. In some embodiments, the mammal is human.

As used herein, the terms “pharmaceutical formulation” or “pharmaceutical composition” refer to a preparation which is in such a form as to permit the biological activity of the active ingredient(s) to be effective, and which contains no additional components which are unacceptably toxic to a subject to which the composition or formulation would be administered. “Pharmaceutically acceptable” excipients (e.g., vehicles, additives) are those which can reasonably be administered to a subject mammal to provide an effective dose of the active ingredient(s) employed.

As used herein, an “effective amount” is at least the minimum amount required to affect a measurable improvement or prevention of one or more symptoms of a particular disorder. An “effective amount” may vary according to factors such as the disease state, age, sex, and weight of the patient. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications used to treat symptoms of the disease, delaying the progression of the disease, and/or prolonging survival. An effective amount can be administered in one or more administrations. For purposes of the present disclosure, an effective amount of a recombinant polynucleotide, gene delivery vehicle comprising the recombinant polynucleotide, pharmaceutical composition, and/or medicament is an amount sufficient to accomplish prophylactic or therapeutic treatment either directly or indirectly. As is understood in the clinical context, an effective amount of a recombinant polynucleotide, gene delivery vehicle comprising the recombinant polynucleotide, pharmaceutical composition, and/or medicament may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “treatment” refers to clinical intervention designed to alter the natural course of the individual or cell being treated during the course of clinical pathology. Desirable effects of treatment include decreasing the rate of disease/disorder/defect progression, ameliorating, or palliating the disease/disorder/defect state, and remission or improved prognosis.

As used herein, the term “delaying progression of” a disease/disorder/defect refers to deferring, hindering, slowing, retarding, stabilizing, and/or postponing development of the disease/disorder/defect. This delay can be of varying lengths or time, depending on the history of the disease/disorder/defect and/or the individual being treated. As is evident to one of ordinary skill in the art, a sufficient or significant delay can, in effect, encompass prevention, in that the individual does not develop the disease.

Throughout the present disclosure, various aspects are presented in a range format. It should be understood that the description in range format is merely for convenience and brevity and should not be construed as an inflexible limitation on the scope of the present disclosure. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the present disclosure. The upper and lower limits of these smaller ranges may independently be comprised in the smaller ranges, and are also encompassed within the present disclosure, subject to any specifically excluded limit in the stated range. Where the stated range comprises one or both of the limits, ranges excluding either or both of those comprised limits are also comprised in the present disclosure. This applies regardless of the breadth of the range.

### III. POLYNUCLEOTIDES

Polynucleotides Encoding IL-2 and/or IL-12 Polypeptides

In some embodiments, the present disclosure relates to a first polynucleotide (e.g., a first recombinant polynucleotide, a first isolated polynucleotide) encoding an IL-2 polypeptide and a second polynucleotide (e.g., a second recombinant polynucleotide, a second isolated polynucleotide) encoding an IL-12 polypeptide. Any suitable IL-2 and/or IL-12 polypeptide described herein or known in the art may be encoded by one or more polynucleotides of the present disclosure, including, for example, human IL-2 and/or human IL-12.

In some embodiments, a polynucleotide of the present disclosure comprises the wild-type coding sequence of any IL-2 and/or IL-12 gene described herein or known in the art (including any isoform or splice variant thereof), including, for example, an IL2 gene (see e.g., NCBI Gene ID: 3558; SEQ ID NO: 5), an IL12A gene (see e.g., NCBI Gene ID: 3592; SEQ ID NO: 6), an IL12B gene (see e.g., NCBI Gene ID: 3593; SEQ ID NO: 7), etc. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the human IL-2 and/or IL-12 genes (and/or coding sequences thereof) described herein or known in the art. In some embodiments, a polynucleotide of the present disclosure comprises a codon-optimized variant of the wild-type coding sequence of any IL-2 and/or IL-12 gene described herein or known in the art. In some embodiments, use a of a codon-optimized variant of the coding sequence of a gene increases stability and/or yield of heterologous expression (RNA and/or protein) of the encoded polypeptide in a target cell, as compared to the stability and/or yield of heterologous expression of a corresponding, non-codon-optimized, wild-type sequence. Any suitable method known in the art for performing codon optimization of a sequence of interest for expression in one or more target cells (e.g., one or more human cells) may be used, including, for example, by the methods described by Fath et al. (PLoS One. 2011 Mar. 3; 6 (3): e17596).

In some embodiments, a first polynucleotide of the present disclosure encodes an IL-2 polypeptide. In some embodiments, the IL-2 polypeptide is a human IL-2 polypeptide (see e.g., UniProt accession number: P60568). In some embodiments, the polynucleotide comprises the coding sequence of a wild-type IL2 gene (see e.g., NCBI Gene ID: 3558, SEQ ID NO: 5), or a codon-optimized variant thereof (see e.g., SEQ ID NO: 8). In some embodiments, a polynucleotide encoding an IL-2 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 1. In some embodiments, a polynucleotide encoding an IL-2 polypeptide is a polynucleotide that encodes a polypeptide comprising the amino acid sequence of SEQ ID NO: 1.

In some embodiments, a first polynucleotide encoding an IL-2 polypeptide is a polynucleotide that encodes an N-terminal truncation, a C-terminal truncation, or a fragment of the amino acid sequence of SEQ ID NO: 1. N-terminal truncations, C-terminal truncations, or fragments may comprise at least 10, at least 12, at least 14, at least 16, at least 18, at least 20, at least 30, at least 40, at least 50, at least 75, at least 100, but fewer than 153, consecutive amino acids of SEQ ID NO: 1.

In some embodiments, a second polynucleotide of the present disclosure encodes an IL-12 subunit α polypeptide. In some embodiments, the IL-12 subunit α polypeptide is a human IL-12 subunit α polypeptide (see e.g., UniProt accession number: P29459). In some embodiments, the polynucleotide comprises the coding sequence of a wild-type IL12A gene (see e.g., NCBI Gene ID: 3592, SEQ ID NO: 6), or a codon-optimized variant thereof (see e.g., SEQ ID NO: 9). In some embodiments, a polynucleotide encoding an IL-12 subunit α polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 2. In some embodiments, a polynucleotide encoding an IL-12 subunit α polypeptide is a polynucleotide that encodes a polypeptide comprising the amino acid sequence of SEQ ID NO: 2.

In some embodiments, a second polynucleotide encoding an IL-12 subunit α polypeptide is a polynucleotide that encodes an N-terminal truncation, a C-terminal truncation, or a fragment of the amino acid sequence of SEQ ID NO: 2. N-terminal truncations, C-terminal truncations, or fragments may comprise at least 10, at least 12, at least 14, at least 16, at least 18, at least 20, at least 30, at least 40, at least 50, at least 75, at least 100, at least 200, but fewer than 219, consecutive amino acids of SEQ ID NO: 2.

In some embodiments, a second polynucleotide of the present disclosure encodes an IL-12 subunit β polypeptide. In some embodiments, the IL-12 subunit β polypeptide is a human IL-12 subunit β polypeptide (see e.g., UniProt accession number: P29460). In some embodiments, the polynucleotide comprises the coding sequence of a wild-type IL12B gene (see e.g., NCBI Gene ID: 3593, SEQ ID NO: 7), or a codon-optimized variant thereof (see e.g., SEQ ID NO: 10). In some embodiments, a recombinant polynucleotide encoding an IL-12 subunit β polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 3. In some embodiments, a polynucleotide encoding an IL-12 subunit β polypeptide is a polynucleotide that encodes a polypeptide comprising the amino acid sequence of SEQ ID NO: 3.

In some embodiments, a second polynucleotide encoding an IL-12 subunit β polypeptide is a polynucleotide that encodes an N-terminal truncation, a C-terminal truncation, or a fragment of the amino acid sequence of SEQ ID NO: 3. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, but fewer than 328, consecutive amino acids of SEQ ID NO: 3.

In some embodiments, a polynucleotide encoding an IL-2 polypeptide, a polynucleotide encoding an IL-12 subunit α polypeptide, and/or a polynucleotide encoding an IL-12 subunit β polypeptide is a single contiguous polynucleotide (e.g., a single contiguous DNA, a single contiguous RNA (such as a synthetic mRNA)). In some embodiments, a polynucleotide encoding an IL-2 polypeptide, a polynucleotide encoding an IL-12 subunit α polypeptide, and/or a polynucleotide encoding an IL-12 subunit β polypeptide is a single contiguous polynucleotide contained within a single DNA molecule. In some embodiments, a polynucleotide encoding an IL-2 polypeptide, a polynucleotide encoding an IL-12 subunit α polypeptide, and/or a polynucleotide encoding an IL-12 subunit β polypeptide is a single contiguous polynucleotide contained within a single RNA molecule (e.g., a synthetic RNA molecule). In some embodiments, a polynucleotide encoding an IL-2 polypeptide, a polynucleotide encoding an IL-12 subunit α polypeptide, and/or a polynucleotide encoding an IL-12 subunit β polypeptide is a single contiguous polynucleotide contained within a single mRNA (e.g., a single synthetic mRNA or mmRNA). In some embodiments, the single contiguous polynucleotide encodes an IL-2 polypeptide on a first open reading frame (ORF) and an IL-12 polypeptide on a second open reading frame (ORF). In some embodiments, the two ORFs are separated by an internal ribosomal entry site (IRES).

In some embodiments, the single contiguous polynucleotide encodes an IL-2 polypeptide on a first open reading frame (ORF), an IL-12 subunit α polypeptide on a second open reading frame (ORF), and an IL-12 subunit β polypeptide on a third open reading frame (ORF). In some embodiments, the first, second, and third ORFs are each separated by an IRES.

Examples of suitable IRES's may include, but are not limited to, a virally-derived IRES (e.g. an IRES derived from a poliovirus, rhinovirus, encephalomyocarditis virus, foot-and-mouth disease virus, hepatitis C virus, classic swine fever virus, rous sarcoma virus, human immunodeficiency virus, cricket paralysis virus, Kaposi's sarcoma-associated herpesvirus, etc.) and a cellular mRNA-derived IRES (e.g. an IRES derived from growth factor mRNAs, such as fibroblast growth factor 2, platelet-derived growth factor B, and vascular endothelial growth factor, an IRES derived from transcription factor mRNAs, such as antennapedia, ultrapithoraxm, and NF-κB repressing factor, an IRES derived from oncogene mRNAs, such as c-myc, pim-1, and protein kinase p58PITSLRE, etc.).

In some embodiments, a polynucleotide encoding any of the chimeric polypeptides described herein (e.g., comprising an IL-12 subunit β polypeptide and an IL-12 subunit α polypeptide) is encoded on a single ORF.

In some embodiments, a polynucleotide of the present disclosure encodes any one or more of an Interleukin-2 (IL-2) peptide, an Interleukin-12 subunit alpha (IL-12a) peptide, an Interleukin-12 subunit beta (IL-12B) peptide, and/or any chimeric polypeptides thereof, in any combination. In some embodiments, a chimeric polypeptide comprises a linker polypeptide.

In some embodiments, the linker polypeptide is a cleavable linker polypeptide. Any cleavable linker polypeptide known in the art may be used in the chimeric polypeptides of the present disclosure, including, for example, a T2A linker, a P2A linker, a E2A linker, and F2A linker, etc. In some embodiments, the linker polypeptide is a T2A linker polypeptide. An exemplary nucleic acid sequence encoding a T2A linker polypeptide is provided as SEQ ID NO: 12. An exemplary amino acid sequence of a T2A linker polypeptide is provided as SEQ ID NO: 16. In some embodiments, the linker polypeptide is a P2A linker polypeptide. An exemplary nucleic acid sequence encoding a P2A linker polypeptide is provided as SEQ ID NO: 13. An exemplary amino acid sequence of a P2A linker polypeptide is provided as SEQ ID NO: 17. In some embodiments, the linker polypeptide is an E2A linker polypeptide. An exemplary nucleic acid sequence encoding an E2A linker polypeptide is provided as SEQ ID NO: 14. An exemplary amino acid sequence of an E2A linker polypeptide is provided as SEQ ID NO: 18. In some embodiments, the linker polypeptide is an F2A linker polypeptide. An exemplary nucleic acid sequence encoding an F2A linker polypeptide is provided as SEQ ID NO: 15. An exemplary amino acid sequence of an F2A linker polypeptide is provided as SEQ ID NO: 19. In some embodiments, the linker polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 16-19. In some embodiments, the linker polypeptide comprises a sequence selected from SEQ ID NOS: 16-19.

In some embodiments, the linker polypeptide is a non-cleavable linker polypeptide. Any non-cleavable linker polypeptide known in the art may be used in the chimeric polypeptides of the present disclosure, including, for example, a GGGGSGGGGSGGGGS (SEQ ID NO: 20) linker, a GGSSRSSSSGGGGSGGGG (SEQ ID NO: 21) linker, a GGGGSGGGGSGGGGSGGGGS (SEQ ID NO: 22) linker, a CGGGSGGGGSGGGGS (SEQ ID NO: 23) linker, a SHGGHGGGGSGGGGS (SEQ ID NO: 24) linker, a MGGMSGGGGSGGGGS (SEQ ID NO: 25) linker, a YGGYSGGGGSGGGGS (SEQ ID NO: 26) linker, a WGGYSGGGGSGGGGS (SEQ ID NO: 27) linker, a SVSVGMKPSPRP (SEQ ID NO: 28) linker, a VISNHAGSSRRL (SEQ ID NO: 29) linker, a PWIPTPRPTFTG (SEQ ID NO: 30) linker, a RGRGRGRGRGR (SEQ ID NO: 31) linker, etc. In some embodiments, the linker polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 20-31. In some embodiments, the linker polypeptide comprises a sequence selected from SEQ ID NOS: 20-31.

In some embodiments, a second polynucleotide of the present disclosure encodes an IL-12 subunit α polypeptide and an IL-12 subunit β polypeptide. In some embodiments, the IL-12 subunit α polypeptide is a human IL-12 subunit α polypeptide (see e.g., UniProt accession number: P29459). In some embodiments, the polynucleotide comprises the coding sequence of a wild-type IL12A gene (see e.g., NCBI Gene ID: 3592, SEQ ID NO: 6), or a codon-optimized variant thereof (see e.g., SEQ ID NO: 9). In some embodiments, a polynucleotide encoding an IL-12 subunit α polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 2. In some embodiments, a polynucleotide encoding an IL-12 subunit α polypeptide is a polynucleotide that encodes a polypeptide comprising the amino acid sequence of SEQ ID NO: 2. In some embodiments, the IL-12 subunit β polypeptide is a human IL-12 subunit β polypeptide (see e.g., UniProt accession number: P29460). In some embodiments, the polynucleotide comprises the coding sequence of a wild-type IL12B gene (see e.g., NCBI Gene ID: 3593, SEQ ID NO: 7), or a codon-optimized variant thereof (see e.g., SEQ ID NO: 10). In some embodiments, a recombinant polynucleotide encoding an IL-12 subunit β polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 3. In some embodiments, a polynucleotide encoding an IL-12 subunit β polypeptide is a polynucleotide that encodes a polypeptide comprising the amino acid sequence of SEQ ID NO: 3. In some embodiments, the IL-as subunit α and β polypeptides are separated by a linker polypeptide.

In some embodiments, a second polynucleotide of the present disclosure encodes a chimeric polypeptide comprising an IL-12 subunit β polypeptide and an IL-12 subunit α polypeptide. In some embodiments, the chimeric polypeptide further comprises a linker polypeptide linking the IL-12 subunit β polypeptide and the IL-12 subunit α polypeptide. In some embodiments, the chimeric polypeptide comprises, from N-terminus to C-terminus, the IL-12 subunit α polypeptide-the linker polypeptide-the IL-12 subunit β polypeptide. In some embodiments, the chimeric polypeptide comprises, from N-terminus to C-terminus, the IL-12 subunit β polypeptide-the linker polypeptide-the IL-12 subunit α polypeptide. An exemplary nucleic acid sequence encoding a chimeric polypeptide comprising a human IL12 subunit β polypeptide, a linker polypeptide, and a human IL12 subunit α polypeptide is provided as SEQ ID NO: 11. An exemplary amino acid sequence of a chimeric polypeptide comprising a human IL12 subunit β polypeptide, a linker polypeptide, and a human IL12 subunit α polypeptide is provided as SEQ ID NO: 4. In some embodiments, a polynucleotide of the present disclosure encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 4. In some embodiments, a polynucleotide of the present disclosure encodes a polypeptide comprising the amino acid sequence of SEQ ID NO: 4.

In some embodiments, a polynucleotide of the present disclosure does not encode an Interleukin-4 (IL-4) polypeptide. In some embodiments, a polynucleotide of the present disclosure does not encode an Interleukin-10 (IL-10) polypeptide. In some embodiments, a polynucleotide of the present disclosure does not encode a Granulocyte-Macrophage Colony-Stimulating Factor (GM-CSF) polypeptide. In some embodiments, a polynucleotide of the present disclosure does not encode an Interleukin-4 (IL-4) polypeptide, an Interleukin-10 (IL-10) polypeptide, and/or a Granulocyte-Macrophage Colony-Stimulating Factor (GM-CSF) polypeptide. In some embodiments, the first polynucleotide encoding an IL-2 polypeptide and/or the second polynucleotide encoding an IL-12 polypeptide encodes one or more additional polypeptides. In some embodiments, the first polynucleotide encoding an IL-2 polypeptide and/or the second polynucleotide encoding an IL-12 polypeptide does not encode any additional polypeptides. In some embodiments, the first polynucleotide encoding an IL-2 polypeptide and/or the second polynucleotide encoding an IL-12 polypeptide encodes one or more additional cytokines and/or chemokines. In some embodiments, the first polynucleotide encoding an IL-2 polypeptide and/or the second polynucleotide encoding an IL-12 polypeptide does not encode any additional cytokines and/or chemokines.

A polynucleotide of the present disclosure (e.g., a recombinant polynucleotide) encoding a polypeptide (e.g., an IL-2 and/or an IL-12 polypeptide) may further encode additional coding and non-coding sequences. Examples of additional coding and non-coding sequences may include, but are not limited to, sequences encoding additional polypeptide tags (e.g., encoded in-frame with the polypeptide in order to produce a fusion protein), introns (e.g., native, modified, or heterologous introns), 5′ and/or 3′ UTRs (e.g., native, modified, or heterologous 5′ and/or 3′ UTRs), and the like. Examples of suitable polypeptide tags may include, but are not limited, to any combination of purification tags, such as his-tags, flag-tags, maltose binding protein and glutathione-S-transferase tags, detection tags, such as tags that may be detected photometrically (e.g., green fluorescent protein, red fluorescent protein, etc.) and tags that have a detectable enzymatic activity (e.g., alkaline phosphatase, etc.), tags containing secretory sequences, signal sequences, leader sequences, and/or stabilizing sequences, protease cleavage sites (e.g., furin cleavage sites, TEV cleavage sites, Thrombin cleavage sites, etc.), and the like. In some embodiments, the 5′ and/or 3′UTRs increase the stability, localization, and/or translational efficiency of the polynucleotides. In some embodiments, the 5′ and/or 3′UTRs improve the level and/or duration of protein expression. In some embodiments, the 5′ and/or 3′UTRs include elements (e.g., one or more miRNA binding sites, etc.) that may block or reduce off-target expression (e.g., inhibiting expression in specific cell types (e.g., neuronal cells), at specific times in the cell cycle, at specific developmental stages, etc.). In some embodiments, the 5′ and/or 3′UTRs include elements (e.g., one or more miRNA binding sites, etc.) that may enhance expression of the encoded polypeptide in specific cell types.

In some embodiments, a polynucleotide of the present disclosure (e.g., a recombinant polynucleotide) encoding a polypeptide (e.g., an IL-2 and/or an IL-12 polypeptide) is operably linked to one or more (e.g., one or more, two or more, three or more, four or more, five or more, ten or more, etc.) regulatory sequences. The term “regulatory sequence” may include enhancers, insulators, promoters, and other expression control elements (e.g., polyadenylation signals). Any suitable enhancer(s) known in the art may be used, including, for example, enhancer sequences from mammalian genes (such as globin, elastase, albumin, α-fetoprotein, insulin and the like), enhancer sequences from a eukaryotic cell virus (such as SV40 enhancer on the late side of the replication origin (bp 100-270), the cytomegalovirus early promoter enhancer, the polyoma enhancer on the late side of the replication origin, adenovirus enhancers, and the like), and any combinations thereof. Any suitable insulator(s) known in the art may be used, including, for example, HSV chromatin boundary (CTRL/CTCF-binding/insulator) elements CTRL1 and/or CTRL2, chicken hypersensitive site 4 insulator (cHS4), human HNRPA2B1-CBX3 ubiquitous chromatin opening element (UCOE), the scaffold/matrix attachment region (S/MAR) from the human interferon beta gene (IFNB1), and any combinations thereof. Any suitable promoter (e.g., suitable for transcription in mammalian host cells) known in the art may be used, including, for example, promoters obtained from the genomes of viruses (such as polyoma virus, fowlpox virus, adenovirus (such as Adenovirus 2), bovine papilloma virus, avian sarcoma virus, cytomegalovirus, a retrovirus, hepatitis-B virus, Simian Virus 40 (SV40), and the like), promoters from heterologous mammalian genes (such as the actin promoter (e.g., the β-actin promoter), a ubiquitin promoter (e.g., a ubiquitin C (UbC) promoter), a phosphoglycerate kinase (PGK) promoter, an immunoglobulin promoter, from heat-shock promoters, and the like), promoters from homologous mammalian genes, synthetic promoters (such as the CAG promoter), and any combinations thereof, provided such promoters are compatible with the host cells. Regulatory sequences may include those which direct constitutive expression of a nucleic acid, as well as tissue-specific regulatory and/or inducible or repressible sequences.

In some embodiments, a polynucleotide of the present disclosure (e.g., a recombinant polynucleotide) is operably linked to one or more heterologous promoters. In some embodiments, the one or more heterologous promoters are one or more of constitutive promoters, tissue-specific promoters, temporal promoters, spatial promoters, inducible promoters, and repressible promoters. In some embodiments, the one or more heterologous promoters are one or more of the human cytomegalovirus (HCMV) immediate early promoter, the human elongation factor-1 (EF1) promoter, the human β-actin promoter, the human UbC promoter, the human PGK promoter, the synthetic CAG promoter, and any combinations thereof.

In some embodiments, a polynucleotide of the present disclosure (e.g., a recombinant polynucleotide) encoding a polypeptide (e.g., an IL-2 and/or an IL-12 polypeptide) expresses the polypeptide when the polynucleotide is delivered into one or more target cells of a subject (e.g., one or more cancer cells, one or more healthy (non-cancerous) cells, one or more cells of the respiratory tract, airway, lungs, etc. of the subject). In some embodiments, expression of the polypeptide (e.g., an IL-2 and/or an IL-12 polypeptide) enhances, increases, augments, and/or supplements the levels, function, and/or activity of the polypeptide in one or more target cells of a subject (e.g., as compared to prior to expression of the polypeptide, as compared to levels of the endogenous polypeptide expressed in the cell, etc.). In some embodiments, expression of the polypeptide (e.g., an IL-2 and/or an IL-12 polypeptide) provides prophylactic, palliative, or therapeutic relief of one or more signs or symptoms of cancer (e.g., solid tumor, hematologic cancer, bladder cancer, brain cancer, breast cancer, colon cancer, gastric cancer, glioma, head cancer, leukemia, liver cancer, lung cancer, lymphoma, myeloma, neck cancer, ovarian cancer, melanoma, pancreatic cancer, renal cancer, salivary cancer, skin cancer, stomach cancer, thymic epithelial cancer, thyroid cancer, etc.) in a subject (e.g., as compared to prior to expression of the polypeptide).

In some embodiments, a polynucleotide of the present disclosure (e.g., a recombinant polynucleotide) does not comprise the coding sequence of (e.g., a transgene encoding) a Collagen alpha-1 (VII) chain polypeptide (COL7). In some embodiments, a polynucleotide of the present disclosure does not comprise the coding sequence of (e.g., a transgene encoding) a Lysyl hydroxylase 3 polypeptide (LH3). In some embodiments, a polynucleotide of the present disclosure does not comprise the coding sequence of (e.g., a transgene encoding) a Keratin type | cytoskeletal 17 polypeptide (KRT17). In some embodiments, a polynucleotide of the present disclosure does not comprise the coding sequence of (e.g., a transgene encoding) a transglutaminase (TGM) polypeptide (e.g., a human transglutaminase polypeptide such as a human TGM1 polypeptide and/or a human TGM5 polypeptide). In some embodiments, a polynucleotide of the present disclosure does not comprise the coding sequence of (e.g., a transgene encoding) a cosmetic protein (e.g., collagen proteins, fibronectins, elastins, lumicans, vitronectins/vitronectin receptors, laminins, neuromodulators, fibrillins, additional dermal extracellular matrix proteins, etc.). In some embodiments, a polynucleotide of the present disclosure does not comprise the coding sequence of (e.g., a transgene encoding) an antibody (e.g., a full-length antibody, an antibody fragment, etc.). In some embodiments, a polynucleotide of the present disclosure does not comprise the coding sequence of (e.g., a transgene encoding) a Serine Protease Inhibitor Kazal-type (SPINK) polypeptide (e.g., a human SPINK polypeptide, such as a SPINK5 polypeptide). In some embodiments, a polynucleotide of the present disclosure does not comprise the coding sequence of (e.g., a transgene encoding) a filaggrin or filaggrin 2 polypeptide (e.g., a human filaggrin or filaggrin 2 polypeptide). In some embodiments, a polynucleotide of the present disclosure does not comprise the coding sequence of (e.g., a transgene encoding) a Cystic Fibrosis Transmembrane Conductance Regulator (CFTR) polypeptide (e.g., a human CFTR polypeptide). In some embodiments, a polynucleotide of the present disclosure does not comprise the coding sequence of (e.g., a transgene encoding) an ichthyosis-associated polypeptide (e.g., an ATP-binding cassette sub-family A member 12 polypeptide, a 1-acylglycerol-3-phosphate O-acyltransferase ABHD5 polypeptide, an Aldehyde dehydrogenase family 3 member A2 polypeptide, an Arachidonate 12-lipoxygenase 12R-type polypeptide, a Hydroperoxide isomerase ALOXE3 polypeptide, an AP-1 complex subunit sigma-1A polypeptide, an Arylsulfatase E polypeptide, a Caspase-14 polypeptide, a Corneodesmosin polypeptide, a Ceramide synthase 3 polypeptide, a Carbohydrate sulfotransferase 8 polypeptide, a Claudin-1 polypeptide, a Cystatin-A polypeptide, a Cytochrome P450 4F22 polypeptide, a 3-beta-hydroxysteroid-Delta (8), Delta (7)-isomerase polypeptide, an Elongation of very long chain fatty acids protein 4 polypeptide, a Filaggrin polypeptide, a Filaggrin 2 polypeptide, a Gap junction beta-2 polypeptide, a Gap junction beta-3 polypeptide, a Gap junction beta-4 polypeptide, a Gap junction beta-6 polypeptide, a 3-ketodihydrosphingosine reductase polypeptide, a Keratin, type II cytoskeletal 1 polypeptide, a Keratin, type II cytoskeletal 2 epidermal polypeptide, a Keratin, type I cytoskeletal 9 polypeptide, a Keratin, type I cytoskeletal 10 polypeptide, a Lipase member N polypeptide, a Loricrin polypeptide, a Membrane-bound transcription factor site-2 protease polypeptide, a Magnesium transporter NIPA4 polypeptide, a Sterol-4-alpha-carboxylate 3-dehydrogenase, decarboxylating polypeptide, a Peroxisomal targeting signal 2 receptor polypeptide, a D-3-phosphoglycerate dehydrogenase polypeptide, a Phytanoyl-CoA dioxygenase, peroxisomal polypeptide, Patatin-like phospholipase domain-containing protein 1 polypeptide, a Proteasome maturation protein polypeptide, a Phosphoserine aminotransferase polypeptide, a Short-chain dehydrogenase/reductase family 9C member 7 polypeptide, a Serpin B8 polypeptide, a Long-chain fatty acid transport protein 4 polypeptide, a Synaptosomal-associated protein 29 polypeptide, a Suppressor of tumorigenicity 14 protein polypeptide, a Steryl-sulfatase polypeptide, a Vacuolar protein sorting-associated protein 33B polypeptide, and a CAAX prenyl protease 1 homolog polypeptide). In some embodiments, a polynucleotide of the present disclosure does not comprise the coding sequence of (e.g., a transgene encoding) a Collagen alpha-1 (VII) chain polypeptide, a Lysyl hydroxylase 3 polypeptide, a Keratin type I cytoskeletal 17 polypeptide, and/or any chimeric polypeptides thereof. In some embodiments, a polynucleotide of the present disclosure does not comprise the coding sequence of (e.g., a transgene encoding) a Collagen alpha-1 (VII) chain polypeptide, a Lysyl hydroxylase 3 polypeptide, a Keratin type I cytoskeletal 17 polypeptide, a transglutaminase (TGM) polypeptide, a filaggrin polypeptide, a cosmetic protein, an antibody, a SPINK polypeptide, a CFTR polypeptide, an ichthyosis-associated polypeptide, an Alpha-1-antitrypsin polypeptide, a Sodium-dependent phosphate transport protein 2B polypeptide, a Dynein heavy chain 5 axonemal polypeptide, a Dynein heavy chain 11 axonemal polypeptide, a Coiled-coil domain-containing protein 39 polypeptide, a Dynein intermediate chain 1 axonemal polypeptide, a Coiled-coil domain-containing protein 40 polypeptide, a Coiled-coil domain containing protein 103 polypeptide, a Sperm-associated antigen 1 polypeptide, a Zinc finger MYND domain-containing protein 10 polypeptide, an Armadillo repeat containing protein 4 polypeptide, a Coiled-coil domain-containing protein 151 polypeptide, a Dynein intermediate chain 2 axonemal polypeptide, a Radial spoke head 1 homolog polypeptide, a Coiled-coil domain-containing protein 114 polypeptide, a Radial spoke head protein 4 homolog A polypeptide, a Dynein assembly factor 1 axonemal polypeptide, a Dynein assembly factor 2 axonemal polypeptide, a Leucine-rich repeat-containing protein 6 polypeptide, a Pulmonary surfactant-associated protein B polypeptide, a Pulmonary surfactant-associated protein C polypeptide, a Homeobox protein Nkx-2.1 polypeptide, an ATP-binding cassette sub-family A member 3 polypeptide, a Cytokine receptor common subunit beta polypeptide, a Granulocyte-macrophage colony-stimulating factor receptor subunit alpha polypeptide, a Bone morphogenetic protein receptor type-2 polypeptide, a Sarcoplasmic/endoplasmic reticulum calcium ATPase 2 polypeptide, a serine/threonine-protein kinase receptor R3 polypeptide, an Endoglin polypeptide, a Mothers against decapentaplegic homolog 9 polypeptide, a Caveolin-1 polypeptide, a Potassium channel subfamily K member 3 polypeptide, an elF-2-alpha kinase GCN2 polypeptide, a Pulmonary surfactant-associated protein A2 polypeptide, a Telomerase reverse transcriptase polypeptide, a Dyskerin polypeptide, a Regulator of telomere elongation helicase 1 polypeptide, a Poly(A)-specific ribonuclease PARN polypeptide, a TERF1-interacting nuclear factor 2 polypeptide, an H/ACA ribonucleoprotein complex non-core subunit NAF1 polypeptide, a Mucin-5B polypeptide, a Desmoplakin polypeptide, a CST complex subunit STN1 polypeptide, a Dipeptidyl peptidase 9 polypeptide, and/or any chimeric polypeptides thereof.

### Synthetic RNA Polynucleotides

In some embodiments, the present disclosure relates to synthetic RNAs containing one or more polynucleotides described herein. In some embodiments, the synthetic RNAs are synthetic mRNAs containing one or more polynucleotides described herein. In some embodiments, the synthetic RNAs comprise a 5′-cap structure. Examples of 5′-cap structures may include, but are not limited to, cap-0, cap-1, cap-2, and cap-3 structures, and any combinations and/or derivatives thereof. In some embodiments, the synthetic RNAs comprise a 3′-poly(A) tail. In some embodiments, the synthetic RNAs comprise one or more 5′ and/or 3′ UTRs flanking the one or more coding sequences contained within the synthetic RNAs. In some embodiments, the 5′ and/or 3′ UTRs increase the stability, localization, and/or translational efficiency of the synthetic RNAs. In some embodiments, the 5′ and/or 3′ UTRs are modified to increase the stability, localization, and/or translational efficiency of the synthetic RNAs. In some embodiments, the 5′ and/or 3′ UTRs improve the level and/or duration of protein expression. In some embodiments, the 5′ and/or 3′ UTRs are modified to improve the level and/or duration of protein expression. In some embodiments, the 5′ and/or 3′UTRs include elements (e.g., miRNA binding sites, etc.) that may limit off-target expression (e.g., inhibiting expression in specific cell types (e.g., neuronal cells), at specific times in the cell cycle, at specific developmental stages, etc.). In some embodiments, the 5′ UTRs comprise a Kozak sequence. In some embodiments, the Kozak sequence is the same or substantially similar to the Kozak consensus sequence. Methods for making synthetic RNAs containing one or more polynucleotides of interest are well known to one of skill in the art.

In some embodiments, the synthetic RNAs of the present disclosure comprise one or more modified ribonucleotides. Examples of modified ribonucleotides may include, but are not limited to, 2-thiouridine, 5-azauridine, pseudouridine, 4-thiouridine, 5-methyluridine, 5-aminouridine, 5-hydroxyuridine, 5-methyl-5-azauridine, 5-amino-5-azauridine, 5-hydroxy-5-azauridine, 5-methylpseudouridine, 5-aminopseudouridine, 5-hydroxypseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine, pseudoisocytidine, N4-methylcytidine, N4-aminocytidine, N4-hydroxycytidine, 5-methylcytidine, 5-aminocytidine, 5-hydroxycytidine, 5-methyl-5-azacytidine, 5-amino-5-azacytidine, 5-hydroxy-5-azacytidine, 5-methylpseudoisocytidine, 5-aminopseudoisocytidine, 5-hydroxypseudoisocytidine, N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine, 2-thio-5-methylcytidine, 2-thio-5-aminocytidine, 2-thio-5-hydroxycytidine, 2-thio-5-methyl-5-azacytidine, e, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine, N4-methyl-5-methyl-5-azacytidine, N4-methyl-5-amino-5-azacytidine, N4-methyl-5-hydroxy-5-azacytidine, N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino-5-azacytidine, N4-amino-5-hydroxy-5-azacytidine, N4-amino-5-methylpseudoisocytidine, N4-amino-5-aminopseudoisocytidine, N4-amino-5-hydroxypseudoisocytidine, N4-hydroxy-5-azacytidine, N4-hydroxypseudoisocytidine, N4-hydroxy-5-methylcytidine, N4-hydroxy-5-aminocytidine, N4-hydroxy-5-hydroxycytidine, N4-hydroxy-5-methyl-5-azacytidine, N4-hydroxy-5-amino-5-azacytidine, N4-hydroxy-5-hydroxy-5-azacytidine, N4-hydroxy-5-methylpseudoisocytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine, 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminopseudoisocytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine, 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy-5-azacytidine, 2-thio-N4-hydroxypseudoisocytidine, 2-thio-N4-hydroxy-5-methylcytidine, N4-hydroxy-5-aminocytidine, 2-thio-N4-hydroxy-5-hydroxycytidine, 2-thio-N4-hydroxy-5-methyl-5-azacytidine, 2-thio-N4-hydroxy-5-amino-5-azacytidine, 2-thio-N4-hydroxy-5-hydroxy-5-azacytidine, 2-thio-N4-hydroxy-5-methylpseudoisocytidine, 2-thio-N4-hydroxy-5-aminopseudoisocytidine, 2-thio-N4-hydroxy-5-hydroxypseudoisocytidine, N6-methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino-8-azaadenosine, N6-amino-7-deaza-8-azaadenosine, N6-hydroxyadenosine, N6-hydroxy-7-deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

In some embodiments, a polynucleotide encoding an IL-2 polypeptide and a polynucleotide encoding an IL-12 polypeptide are contained within two separate synthetic RNA molecules (e.g., two separate synthetic mRNAs). In some embodiments, a polynucleotide encoding an IL-2 polypeptide, a polynucleotide encoding an IL-12 subunit α polypeptide, and/or a polynucleotide encoding an IL-12 subunit β polypeptide are contained within three separate synthetic RNA molecules (e.g., three separate synthetic mRNAs).

### IV. RECOMBINANT NUCLEIC ACIDS

Certain aspects of the present disclosure relate to recombinant nucleic acids comprising any one or more of the polynucleotides described herein (e.g., the first recombinant polynucleotide encoding an IL-2 polypeptide and/or the second recombinant polynucleotide encoding an IL-12 polypeptide). In some embodiments, the recombinant nucleic acid is a linear DNA, a circular DNA, a closed ended DNA (ceDNA), an RNA (e.g., a synthetic RNA), an mRNA, and/or an mmRNA. In some embodiments, the recombinant nucleic acid is a recombinant viral genome.

In some embodiments, a recombinant nucleic acid of the present disclosure is a linear DNA. In some embodiments, the linear DNA is a linear expression template (LET). As would be understood by those of skill in the art, a LET may comprise a promoter region, one or more recombinant polynucleotides described herein, and/or a transcriptional terminator. As would be understood by those of skill in the art, a LET may be produced in vitro via polymerase chain reaction (PCR) from, for example, plasmid DNA or genomic DNA.

In some embodiments, a recombinant nucleic acid of the present disclosure is a close ended DNA (ceDNA; e.g., as disclosed in U.S. Patent Application Publication NO. 2021/0059953 and/or U.S. Patent Application Publication NO. 2020/0283794, the contents of which are hereby incorporated by reference in its entirety).

In some embodiments, a recombinant nucleic acid of the present disclosure is a circular DNA. In some embodiments, the circular DNA is a plasmid (e.g., as disclosed in U.S. Patent Application Publication No. 2013/0195800 and U.S. Pat. Nos. 5,693,622, 5,589,466, and 5,580,859, the contents of which are hereby incorporated by reference in its entirety), a cosmid, or a bacterial artificial chromosome (BAC).

In some embodiments, the circular DNA is a plasmid. A plasmid can be in the form of circular double-stranded DNA. In some embodiments, the plasmid may contain autonomously replicating sequences, genome integrating sequences, and/or phage or nucleotide sequences. In some embodiments, the plasmid may be linear, circular, or supercoiled, of a single- or double-stranded DNA or RNA. The plasmids described herein may be derived from any source in which a number of nucleotide sequences have been joined or recombined into a unique construction which is capable of introducing a promoter fragment and DNA sequence for a selected gene product along with appropriate 3′ untranslated sequence into a cell.

In some embodiments, the circular DNA is a cosmid. As would be understood by those of skill in the art, a cosmid is a type of plasmid that contains a Lambda phage cos sequence. Cosmids may contain 37 to 52 kb of DNA. In some embodiments, cosmids may replicate if they have an origin of replication (e.g. an SV40 in mammalian cells). In some embodiments, cosmids may be packaged into phage capsids. In some embodiments, the cosmid is a plasmid with a bacterial oriV, an antibiotic selection marker, and/or a cloning site. In some embodiments, the cosmid may have one or more (e.g. two) cos sites derived from bacteriophage lambda.

In some embodiments, the circular DNA is a bacterial artificial chromosome (BAC). As would be understood by those of skill in the art, a BAC is a DNA construct, based on functional fertility plasmid (e.g. F-plasmid). In some embodiments, the BAC may accommodate an insert size of 150-350 kbp. In some embodiments, the BAC may comprise: an oriS and/or a repE F for plasmid replication and/or regulation of copy number; parA and/or parB which may partition F plasmid DNA into daughter cells during division and may ensure stable maintenance of the BAC; a selection marker; and/or T7 and/or Sp6 for transcription of inserted genes.

In some embodiments, a recombinant nucleic acid of the present disclosure is an RNA, mRNA, and/or mmRNA (e.g., as disclosed in U.S. Pat. Nos. 10,898,574, 10,702,600, and 10,933,127, the contents of which are hereby incorporated by reference in their entirety).

In some embodiments, the RNA, mRNA, and/or mmRNA may enhance the efficiency of protein production, intracellular retention of nucleic acids, and viability of contacted cells, as well as possess reduced immunogenicity.

In some embodiments, the RNA, mRNA, and/or mmRNA may have a length that is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides).

In some embodiments, the RNA, mRNA, and/or mmRNA may contain one or more modified nucleosides (e.g., as described above) which may have useful properties including the lack of a substantial induction of the innate immune system of a cell into which the RNA, mRNA, and/or mmRNA is introduced.

In some embodiments, the modified nucleosides may not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of a nucleic acid such that the function of the nucleic acid is not substantially decreased. A modification may also be a 5′ or 3′ terminal modification. The nucleic acids may contain at a minimum one and at maximum 100% modified nucleotides, or any intervening percentage, such as at least 50% modified nucleotides, at least 80% modified nucleotides, or at least 90% modified nucleotides.

In some embodiments, the modified nucleosides may include modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In some embodiments, the modified nucleosides may include 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In some embodiments, the modified nucleosides may include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2, N2-dimethyl-6-thio-guanosine.

Multiple distinct 5′-cap structures can be used to generate the 5′-cap of the RNA, mRNA, and/or mmRNA described herein. Many chemical cap analogs are used to co-transcriptionally cap a synthetic mRNA molecule. For example, the Anti-Reverse Cap Analog (ARCA) cap contains a 5′-5′-triphosphate guanine-guanine linkage where one guanine contains an N7 methyl group as well as a 3′-O-methyl group. While chemical cap analogs allow for the concomitant capping of an RNA molecule, up to 20% of transcripts remain uncapped and the synthetic cap analog is not identical to an endogenous 5′-cap structure of an authentic cellular mRNA. This may lead to reduced translational competency and reduced cellular stability.

In some embodiments, the RNA, mRNA, and/or mmRNA includes the poly-A tail. In some embodiments, the poly-A tail is designed relative to the length of the overall modified RNA, mRNA, and/or mmRNA. This design may be based on the length of the coding region of the modified RNA, the length of a particular feature or region of the modified RNA (such as the mRNA), or based on the length of the ultimate product expressed from the modified RNA. In this context the poly-A tail may be 10, 20, 30, 40, 50, 60, 70, 80, 90 or 100% greater in length than the modified RNA or feature thereof. The poly-A tail may also be designed as a fraction of the modified RNA to which it belongs. In this context, the poly-A tail may be 1, 2, 3, 4, 5, 6, 7, 8, 9, 10, 20, 30, 40, 50, 60, 70, 80, or 90% or more of the total length of the construct or the total length of the construct minus the poly-A tail.

In some embodiments, a recombinant nucleic acid of the present disclosure is one or more recombinant viral genomes and/or one or more non-viral nucleic acids. In some embodiments, the one or more recombinant viral genomes and/or one or more non-viral nucleic acids comprises a DNA vector or an RNA vector. Generally, vectors suitable to maintain, propagate, and/or express polynucleotides to produce one or more polypeptides in a subject may be used. Examples of suitable vectors may include, for example, plasmids, cosmids, episomes, transposons, and viral vectors (e.g., adenoviral vectors, adeno-associated viral vectors, vaccinia viral vectors, pox viral vectors, bacteriophage vector, alpha viral vector, picornaviral vector, iridoviral vector, Newcastle disease viral vector, baculoviral vector, geminiviral vector, caulimoviral vector, Sindbis-viral vectors, measles vectors, herpes viral vectors, lentiviral vectors, retroviral vectors, etc.). In some embodiments, one or more recombinant viral genomes and/or one or more non-viral nucleic acids is replication competent in a host cell. In some embodiments, the one or more recombinant viral genomes and/or one or more non-viral nucleic acids is replication incompetent in a host cell. In some embodiments, the one or more recombinant viral genomes and/or one or more non-viral nucleic acids can integrate into a host DNA. In some embodiments, the one or more recombinant viral genomes and/or one or more non-viral nucleic acids cannot integrate into a host DNA (e.g., is episomal).

In some embodiments, one or more of the recombinant nucleic acids described herein is a recombinant adenovirus genome. The recombinant adenovirus genome may be a recombinant genome from any member of the Adenoviridae family of viruses known in the art, including, for example, a recombinant Atadenovirus genome, a recombinant Aviadenovirus genome, a recombinant Ichtadenovirus genome, a recombinant Mastadenovirus genome, a recombinant Siadenovirus genome, a recombinant Testadenovirus genome, and any combinations or derivatives thereof. In some embodiments, the recombinant adenovirus genome comprises one or more (e.g., one or more, two or more, three or more, four or more, five or more, six or more, seven or more, eight or more, nine or more, ten or more, etc.) inactivating mutations. As used herein, an “inactivating mutation” may refer to any mutation that results in a gene or regulon product (RNA or protein) having reduced, undetectable, or eliminated quantity and/or function (e.g., as compared to a corresponding sequence lacking the inactivating mutation). Examples of inactivating mutations may include, but are not limited to, deletions, insertions, point mutations, and rearrangements in transcriptional control sequences (promoters, enhancers, insulators, etc.) and/or coding sequences of a given gene or regulon. Any suitable method of measuring the quantity of a gene or regulon product known in the art may be used, including, for example, qPCR, Northern blots, RNAseq, western blots, ELISAs, etc. In some embodiments, the one or more inactivating mutations are in one or more (e.g., one or more, two or more, three or more, four or more, five or more, six or more, seven or more, eight or more, nine or more, ten or more, etc.) adenovirus genes.

In some embodiments, the recombinant Atadenovirus genome may be a recombinant Bovine atadenovirus D genome, a recombinant Bovine atadenovirus E genome, a recombinant Deer atadenovirus A genome, a recombinant Duck atadenovirus A genome, a recombinant Lizard atadenovirus A genome, a recombinant Lizard atadenovirus B genome, a recombinant Ovine atadenovirus D genome, a recombinant Possum atadenovirus A genome, a recombinant Psittacine atadenovirus A genome, a recombinant Snake atadenovirus A genome, and any combinations or derivatives thereof.

In some embodiments, the recombinant Aviadenovirus genome may be a recombinant Duck aviadenovirus B genome, a recombinant Falcon aviadenovirus A genome, a recombinant Fowl aviadenovirus A genome, a recombinant Fowl aviadenovirus B genome, a recombinant Fowl aviadenovirus C genome, a recombinant Fowl aviadenovirus D genome, a recombinant Fowl aviadenovirus E genome, a recombinant Goose aviadenovirus A genome, a recombinant Pigeon aviadenovirus A genome, a recombinant Pigeon aviadenovirus B genome, a recombinant Psittacine aviadenovirus B genome, a recombinant Psittacine aviadenovirus C genome, a recombinant Turkey aviadenovirus B genome, a recombinant Turkey aviadenovirus C genome, a recombinant Turkey aviadenovirus D genome, and any combinations or derivatives thereof.

In some embodiments, the recombinant Ichtadenovirus genome may be a recombinant Sturgeon ichtadenovirus A.

In some embodiments, the recombinant Mastadenovirus genome may be a recombinant Bat mastadenovirus A genome, a recombinant Bat mastadenovirus B genome, a recombinant Bat mastadenovirus C genome, a recombinant Bat mastadenovirus D genome, a recombinant Bat mastadenovirus E genome, a recombinant Bat mastadenovirus F genome, a recombinant Bat mastadenovirus G genome, a recombinant Bat mastadenovirus H genome, a recombinant Bat mastadenovirus I genome, a recombinant Bat mastadenovirus J genome, a recombinant Bovine mastadenovirus A genome, a recombinant Bovine mastadenovirus B genome, a recombinant Bovine mastadenovirus C genome, a recombinant Canine mastadenovirus A genome, a recombinant Deer mastadenovirus B genome, a recombinant Dolphin mastadenovirus A genome, a recombinant Dolphin mastadenovirus B genome, a recombinant Equine mastadenovirus A genome, a recombinant Equine mastadenovirus B genome, a recombinant Guinea pig mastadenovirus A genome, a recombinant Human mastadenovirus A genome, a recombinant Human mastadenovirus B genome, a recombinant Human mastadenovirus C genome, a recombinant Human mastadenovirus D genome, a recombinant Human mastadenovirus E genome, a recombinant Human mastadenovirus F genome, a recombinant Human mastadenovirus G genome, a recombinant Murine mastadenovirus A genome, a recombinant Murine mastadenovirus B genome, a recombinant Murine mastadenovirus C genome, a recombinant Ovine mastadenovirus A genome, a recombinant Ovine mastadenovirus B genome, a recombinant Ovine mastadenovirus C genome, a recombinant Platyrrhini mastadenovirus A genome, a recombinant Polar bear mastadenovirus A genome, a recombinant Porcine mastadenovirus A genome, a recombinant Porcine mastadenovirus B genome, a recombinant Porcine mastadenovirus C genome, a recombinant Sea lion mastadenovirus A genome, a recombinant Simian mastadenovirus A genome, a recombinant Simian mastadenovirus B genome, a recombinant Simian mastadenovirus C genome, a recombinant Simian mastadenovirus D genome, a recombinant Simian mastadenovirus E genome, a recombinant Simian mastadenovirus F genome, a recombinant Simian mastadenovirus G genome, a recombinant Simian mastadenovirus H genome, a recombinant Simian mastadenovirus I genome, a recombinant Skunk mastadenovirus A genome, a recombinant Squirrel mastadenovirus A genome, a recombinant Tree shrew mastadenovirus A genome, and any combinations or derivatives thereof.

In some embodiments, the recombinant Mastadenovirus genome may be the recombinant Human mastadenovirus A genome, the recombinant Human mastadenovirus B genome, the recombinant Human mastadenovirus C genome, the recombinant Human mastadenovirus D genome, the recombinant Human mastadenovirus E genome, the recombinant Human mastadenovirus F genome, or the recombinant Human mastadenovirus G genome, and any combinations or derivatives thereof.

In some embodiments, the recombinant Human mastadenovirus A genome may be a recombinant Human mastadenovirus A serotype 12, 18, 31, and any combinations or derivatives thereof.

In some embodiments, the recombinant Human mastadenovirus B genome may be a recombinant Human mastadenovirus B serotype 3, 7, 11, 14, 16, 21, 34, 35, 50, 55, and any combinations or derivatives thereof.

In some embodiments, the recombinant Human mastadenovirus C genome may be a recombinant Human mastadenovirus C serotype 1, 2, 5, 6, 57, and any combinations or derivatives thereof.

In some embodiments, the recombinant Human mastadenovirus D genome may be a recombinant Human mastadenovirus D serotype 8, 9, 10, 13, 15, 17, 19, 20, 22, 23, 24, 25, 26, 27, 28, 29, 30, 32, 33, 36, 37, 38, 39, 42, 43, 44, 45, 46, 47, 48, 49, 51, 53, 54, 56, 58, 59, 60, 62, 63, 64, 65, 67, 69, 70, 71, 72, 73, 74, 75, and any combinations or derivatives thereof.

In some embodiments, the recombinant Human mastadenovirus E genome may be a recombinant Human mastadenovirus E serotype 4.

In some embodiments, the recombinant Human mastadenovirus F genome may be a recombinant Human mastadenovirus F serotype 40, 41, and any combinations or derivatives thereof.

In some embodiments, the recombinant Human mastadenovirus G genome may be a recombinant Human mastadenovirus G serotype 52.

In some embodiments, the recombinant Siadenovirus genome may be a recombinant Frog siadenovirus A genome, a recombinant Great tit siadenovirus A genome, a recombinant Penguin siadenovirus A genome, a recombinant Psittacine siadenovirus D genome, a recombinant Psittacine siadenovirus E genome, a recombinant Raptor siadenovirus A genome, a recombinant Skua siadenovirus A genome, a recombinant Turkey siadenovirus A genome, and any combinations or derivatives thereof.

In some embodiments, the recombinant adenovirus genome may be the recombinant Testadenovirus genome.

In some embodiments, one or more of the recombinant nucleic acids described herein are a recombinant retrovirus genome. The recombinant retrovirus genome may be a recombinant genome from any member of the Retroviridae family of viruses known in the art, including, for example, a recombinant Alpharetrovirus genome, a recombinant Betaretrovirus genome, a recombinant Gammaretrovirus genome, a recombinant Deltaretrovirus genome, a recombinant Epsilonretrovirus genome, a recombinant Lentivirus genome, a recombinant Bovispumavirus genome, a recombinant Equispumavirus genome, a recombinant Felispumavirus genome, a recombinant Prosimiispumavirus genome, a recombinant Simiispumavirus genome, and any combinations or derivatives thereof. In some embodiments, the recombinant retrovirus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) retrovirus genes.

In some embodiments, the recombinant Alpharetrovirus genome may be a recombinant Avian carcinoma Mill Hill virus 2 genome, a recombinant Avian leukosis virus genome, a recombinant Avian myeloblastosis virus genome, a recombinant Avian myelocytomatosis virus 29 genome, a recombinant Avian sarcoma virus CT10 genome, a recombinant Fujinami sarcoma virus genome, a recombinant Rous sarcoma virus genome, a recombinant UR2 sarcoma virus genome, a recombinant Y73 sarcoma virus genome, and any combinations or derivatives thereof.

In some embodiments, the recombinant Betaretrovirus genome may be a recombinant Jaagsiekte sheep retrovirus genome, a recombinant Langur virus genome, a recombinant Mason-Pfizer monkey virus genome, a recombinant Mouse mammary tumor virus genome, a recombinant Squirrel monkey retrovirus genome, and any combinations or derivatives thereof.

In some embodiments, the recombinant Gammaretrovirus genome may be a recombinant Chick syncytial virus genome, a recombinant Feline leukemia virus genome, a recombinant Finkel-Biskis-Jinkins murine sarcoma virus genome, a recombinant Gardner-Arnstein feline sarcoma virus genome, a recombinant Gibbon ape leukemia virus genome, a recombinant Guinea pig type-C oncovirus genome, a recombinant Hardy-Zuckerman feline sarcoma virus genome, a recombinant Harvey murine sarcoma virus genome, a recombinant Kirsten murine sarcoma virus genome, a recombinant Koala retrovirus genome, a recombinant Moloney murine sarcoma virus genome, a recombinant Murine leukemia virus genome, a recombinant Porcine type-C oncovirus genome, a recombinant Reticuloendotheliosis virus genome, a recombinant Snyder-Theilen feline sarcoma virus genome, a recombinant Trager duck spleen necrosis virus genome, a recombinant Viper retrovirus genome, a recombinant Woolly monkey sarcoma virus genome, and any combinations or derivatives thereof.

In some embodiments, the recombinant Deltaretrovirus genome may be a recombinant Bovine leukemia virus genome, a recombinant Primate T-lymphotropic virus 1 genome, a recombinant Primate T-lymphotropic virus 2 genome, a recombinant Primate T-lymphotropic virus 3 genome, and any combinations or derivatives thereof.

In some embodiments, the recombinant Epsilonretrovirus genome may be a recombinant Walleye dermal sarcoma virus genome, a recombinant Walleye epidermal hyperplasia virus 1 genome, a recombinant Walleye epidermal hyperplasia virus 2 genome, and any combinations or derivatives thereof.

In some embodiments, the recombinant Lentivirus genome may be a recombinant Bovine immunodeficiency virus genome, a recombinant Caprine arthritis encephalitis virus genome, a recombinant Equine infectious anemia virus genome, a recombinant Feline immunodeficiency virus genome, a recombinant Human immunodeficiency virus 1 genome, a recombinant Human immunodeficiency virus 2 genome, a recombinant Jembrana disease virus genome, a recombinant Puma lentivirus genome, a recombinant Simian immunodeficiency virus genome, a recombinant Visna-maedi virus genome, and any combinations or derivatives thereof.

In some embodiments, the recombinant retrovirus genome may be the recombinant Bovispumavirus genome,

In some embodiments, the recombinant retrovirus genome may be the recombinant Equispumavirus genome.

In some embodiments, the recombinant retrovirus genome may be the recombinant Felispumavirus genome.

In some embodiments, the recombinant retrovirus genome may be the recombinant Prosimiispumavirus genome.

In some embodiments, the recombinant Simiispumavirus genome may be a recombinant Simian foamy virus genome, a recombinant Human foamy virus genome, and any combinations or derivatives thereof.

In some embodiments, one or more recombinant nucleic acids described herein are a recombinant adeno-associated virus genome. The recombinant adeno-associated virus genome may be a recombinant genome from any member of the Dependoparvovirus family of viruses known in the art, including, for example, a recombinant Adeno-associated dependoparvovirus A genome, a recombinant Adeno-associated dependoparvovirus B genome, and any combinations or derivatives thereof. In some embodiments, the recombinant adeno-associated virus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) adeno-associated virus genes.

In some embodiments, the recombinant adeno-associated virus (AAV) genome may be a recombinant AAV serotype 1 genome, a recombinant AAV serotype 2 genome, a recombinant AAV serotype 3 genome, a recombinant AAV serotype 4 genome, a recombinant AAV serotype 5 genome, a recombinant AAV serotype 6 genome, a recombinant AAV serotype 7 genome, a recombinant AAV serotype 8 genome, a recombinant AAV serotype 9 genome, a recombinant AAV serotype 10 genome. a recombinant AAV serotype 11 genome, and any combinations or derivatives thereof.

In some embodiments, one or more of the recombinant nucleic acids described herein are a recombinant herpes simplex virus (HSV) amplicon. Herpes virus amplicons, including the structural features and methods of making the same, are generally known to one of ordinary skill in the art (see e.g., de Silva S. and Bowers W. “Herpes Virus Amplicon Vectors”. Viruses 2009, 1, 594-629). In some embodiments, the herpes simplex virus amplicon is an HSV-1 amplicon. In some embodiments, the herpes simplex virus amplicon is an HSV-1 hybrid amplicon. Examples of HSV-1 hybrid amplicons may include, but are not limited to, HSV/AAV hybrid amplicons, HSV/EBV hybrid amplicons, HSV/EBV/RV hybrid amplicons, and/or HSV/Sleeping Beauty hybrid amplicons. In some embodiments, the amplicon is an HSV/AAV hybrid amplicon. In some embodiments, the amplicon is an HSV/Sleeping Beauty hybrid amplicon.

In some embodiments, one or more of the recombinant nucleic acids described herein are a recombinant herpes virus genome. The recombinant herpes virus genome may be a recombinant genome from any member of the Herpesviridae family of DNA viruses known in the art, including, for example, a recombinant herpes simplex virus genome, a recombinant varicella zoster virus genome, a recombinant human cytomegalovirus genome, a recombinant herpesvirus 6A genome, a recombinant herpesvirus 6B genome, a recombinant herpesvirus 7 genome, a recombinant Epstein-Barr virus genome, a recombinant Kaposi's sarcoma-associated herpesvirus genome, and any combinations or derivatives thereof. In some embodiments, the recombinant herpes virus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) herpes virus genes.

In some embodiments, the recombinant nucleic acid is a recombinant herpes simplex virus (HSV) genome. In some embodiments, the recombinant herpes virus genome is a recombinant herpes simplex virus type 1 (HSV-1) genome, a recombinant herpes simplex virus type 2 (HSV-2) genome, or any derivatives thereof. In some embodiments, the recombinant herpes simplex virus genome is a recombinant HSV-1 genome. In some embodiments, the recombinant HSV-1 genome may be from any HSV-1 strain known in the art, including, for example, strains 17, Ty25, R62, S25, Ku86, S23, R11, Ty148, Ku47, H166_{syn}, 1319-2005, F-13, M-12, 90237, F-17, KOS, 3083-2008, F12g, L2, CD38, H193, M-15, India 2011, 0116209, F-111, 66-207, 2762, 369-2007, 3355, Macintyre, McKrae, 7862, 7-hse, HF10, 1394,2005, 270-2007, OD4, SC16, M-19, 4J1037, 5J1060, J1060, KOS79, 132-1988, 160-1982, H166, 2158-2007, RE, 78326, F18g, F11, 172-2010, H129, F, E4, CJ994, F14g, E03, E22, E10, E06, E11, E25, E23, E35, E15, E07, E12, E14, E08, E19, E13, ATCC 2011, etc. (see e.g., Bowen et al. J Virol. 2019 Apr. 3; 93 (8)). In some embodiments, the recombinant HSV-1 genome is from the KOS strain. In some embodiments, the recombinant HSV-1 genome is not from the McKrae strain.

In some embodiments, one or more of the recombinant nucleic acids described herein are a recombinant poxvirus genome. The recombinant poxvirus genome may be a recombinant genome from any member of the Poxviridae family of viruses known in the art, including, for example, a recombinant smallpox virus genome, a recombinant vaccinia virus genome, a recombinant cowpox virus genome, a recombinant monkeypox virus genome, a recombinant orf virus genome, a recombinant pseudocowpox virus genome, a recombinant bovine papular stomatitis virus genome, a recombinant tanapox virus genome, a recombinant yaba monkey tumor virus genome, a recombinant molluscum contagiosum virus genome, and any combinations or derivatives thereof. In some embodiments, the recombinant poxvirus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) poxvirus genes.

In some embodiments, one or more of the recombinant nucleic acids described herein are a recombinant bacteriophage genome. The recombinant bacteriophage genome may be, for example, a recombinant 186 phage genome, a recombinant Escherichia virus Lambda genome, a recombinant Pseudomonas virus phi6 genome, a recombinant Bacillus virus φ29 genome, a recombinant Escherichia virus φX174 genome, a recombinant Bacteriophage φCb5 genome, a recombinant Escherichia virus G4 genome, a recombinant Escherichia virus M13 genome, a recombinant Emesvirus zinderi genome, a recombinant N4 phage genome, a recombinant Escherichia virus P1 genome, a recombinant Escherichia virus P2 genome, a recombinant Enterobacteria phage P4 genome, a recombinant R17 phage genome, a recombinant Enterobacteria phage T2 genome, a recombinant Escherichia virus T4 genome, a recombinant Escherichia virus T7 genome, a recombinant Bacteriophage T12 genome, and any combinations or derivatives thereof. In some embodiments, the recombinant bacteriophage genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) bacteriophage genes.

In some embodiments, one or more of the recombinant nucleic acids described herein are a recombinant alphavirus genome. The recombinant alphavirus genome may be a recombinant genome from any member of the Alphavirus family of viruses known in the art, including, for example, a recombinant Aura virus genome, a recombinant Barmah Forest virus genome, a recombinant Bebaru virus genome, a recombinant Caaingua virus genome, a recombinant Cabassou virus genome, a recombinant Chikungunya virus genome, a recombinant Eastern equine encephalitis virus genome, a recombinant Eilat virus genome, a recombinant Everglades virus genome, a recombinant Fort Morgan virus genome, a recombinant Getah virus genome, a recombinant Highlands J virus genome, a recombinant Madariaga virus genome, a recombinant Mayaro virus genome, a recombinant Middelburg virus genome, a recombinant Mosso das Pedras virus genome, a recombinant Mucambo virus genome, a recombinant Ndumu virus genome, a recombinant O'nyong'nyong virus genome, a recombinant Pixuna virus genome, a recombinant Rio Negro virus genome, a recombinant Ross River virus genome, a recombinant Salmon pancreas disease virus genome, a recombinant Semliki Forest virus genome, a recombinant Sindbis virus genome, a recombinant Southern elephant seal virus genome, a recombinant Tonate virus genome, a recombinant Trocara virus genome, a recombinant Una virus genome, a recombinant Venezuelan equine encephalitis virus genome, a recombinant Western equine encephalitis virus genome, a recombinant Whataroa virus genome, and any combinations or derivatives thereof. In some embodiments, the recombinant alphavirus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) alphavirus genes.

In some embodiments, one or more of the recombinant nucleic acids described herein are a recombinant picornavirus genome. The recombinant piconavirus genome may be a recombinant genome from any member of the Picornaviridae family of viruses known in the art, including, for example, a recombinant Aalivirus genome, a recombinant Ailurivirus genome, a recombinant Ampivirus genome, a recombinant Anativirus genome, a recombinant Aphthovirus genome, a recombinant Aquamavirus genome, a recombinant Avihepatovirus genome, a recombinant Avisivirus genome, a recombinant Boosepivirus genome, a recombinant Bopivirus genome, a recombinant Caecilivirus genome, a recombinant Cardiovirus genome, a recombinant Cosavirus genome, a recombinant Crahelivirus genome, a recombinant Crohivirus genome, a recombinant Danipivirus genome, a recombinant Dicipivirus genome, a recombinant Diresapivirus genome, a recombinant Enterovirus genome, a recombinant Erbovirus genome, a recombinant Felipivirus genome, a recombinant Fipivirus genome, a recombinant Gallivirus genome, a recombinant Gruhelivirus genome, a recombinant Grusopivirus genome, a recombinant Harkavirus genome, a recombinant Hemipivirus genome, a recombinant Hepatovirus genome, a recombinant Hunnivirus genome, a recombinant Kobuvirus genome, a recombinant Kunsagivirus genome, a recombinant Limnipivirus genome, a recombinant Livupivirus genome, a recombinant Ludopivirus genome, a recombinant Malagasivirus genome, a recombinant Marsupivirus genome, a recombinant Megrivirus genome, a recombinant Mischivirus genome, a recombinant Mosavirus genome, a recombinant Mupivirus genome, a recombinant Myrropivirus genome, a recombinant Orivirus genome, a recombinant Oscivirus genome, a recombinant Parabovirus genome, a recombinant Parechovirus genome, a recombinant Pasivirus genome, a recombinant Passerivirus genome, a recombinant Pemapivirus genome, a recombinant Poecivirus genome, a recombinant Potamipivirus genome, a recombinant Pygoscepivirus genome, a recombinant Rabovirus genome, a recombinant Rafivirus genome, a recombinant Rajidapivirus genome, a recombinant Rohelivirus genome, a recombinant Rosavirus genome, a recombinant Sakobuvirus genome, a recombinant Salivirus genome, a recombinant Sapelovirus genome, a recombinant Senecavirus genome, a recombinant Shanbavirus genome, a recombinant Sicinivirus genome, a recombinant Symapivirus genome, a recombinant Teschovirus genome, a recombinant Torchivirus genome, a recombinant Tottorivirus genome, a recombinant Tremovirus genome, a recombinant Tropivirus genome, and any combinations or derivatives thereof. In some embodiments, the recombinant picornavirus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) picornavirus genes.

In some embodiments, the recombinant Enterovirus genome may be a recombinant Coxsackievirus genome, a recombinant Poliovirus genome, a recombinant Echovirus genome, a recombinant Bovine enterovirus genome, and any combinations or derivatives thereof.

In some embodiments, the recombinant Senecavirus genome may be a recombinant Seneca Valley virus genome.

In some embodiments, the recombinant Cardiovirus genome may be a recombinant Theiler's Murine Encephalomyelitis genome, a recombinant Encephalomyocarditis virus genome, a recombinant Mengovirus genome, and any combinations or derivatives thereof.

In some embodiments, one or more of the recombinant nucleic acids described herein are a recombinant iridovirus genome. The recombinant iridovirus genome may be a recombinant genome from any member of the Iridovirus family of viruses known in the art, including, for example, a recombinant Invertebrate iridescent virus 6 (IIV-6) genome, a recombinant Invertebrate iridescent virus 31 (IIV-31) genome, and any combinations or derivatives thereof. In some embodiments, the recombinant iridovirus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) iridovirus genes.

In some embodiments, one or more of the recombinant nucleic acids described herein are a recombinant Newcastle disease virus genome. In some embodiments, the recombinant Newcastle disease virus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) Newcastle virus genes.

In some embodiments, one or more of the recombinant nucleic acids described herein are a recombinant baculovirus genome. The recombinant baculovirus genome may be a recombinant genome from any member of the Baculoviridae family of viruses known in the art, including, for example, a recombinant alphabaculovirus genome, a recombinant betabaculovirus genome, a recombinant deltabaculovirus genome, a recombinant gammabaculovirus genome, and any combinations or derivatives thereof. In some embodiments, the recombinant baculoviridae genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) baculovirus genes.

In some embodiments, the recombinant alphabaculovirus genome may be a recombinant Adoxophyes honmai nucleopolyhedrovirus genome, a recombinant Agrotis ipsilon multiple nucleopolyhedrovirus genome, a recombinant Agrotis segetum nucleopolyhedrovirus A genome, a recombinant Agrotis segetum nucleopolyhedrovirus B genome, a recombinant Antheraea pernyi nucleopolyhedrovirus genome, a recombinant Anticarsia gemmatalis multiple nucleopolyhedrovirus genome, a recombinant Autographa californica multiple nucleopolyhedrovirus genome, a recombinant Bombyx mori nucleopolyhedrovirus genome, a recombinant Buzura suppressaria nucleopolyhedrovirus genome, a recombinant Catopsilia pomona nucleopolyhedrovirus genome, a recombinant Choristoneura fumiferana DEF multiple nucleopolyhedrovirus genome, a recombinant Choristoneura fumiferana multiple nucleopolyhedrovirus genome, a recombinant Choristoneura murinana nucleopolyhedrovirus genome, a recombinant Choristoneura rosaceana nucleopolyhedrovirus genome, a recombinant Chrysodeixis chalcites nucleopolyhedrovirus genome, a recombinant Chrysodeixis includens nucleopolyhedrovirus genome, a recombinant Clanis bilineata nucleopolyhedrovirus genome, a recombinant Condylorrhiza vestigialis nucleopolyhedrovirus genome, a recombinant Cryptophlebia peltastica nucleopolyhedrovirus genome, a recombinant Cyclophragma undans nucleopolyhedrovirus genome, a recombinant Ectropis obliqua nucleopolyhedrovirus genome, a recombinant Epiphyas postvittana nucleopolyhedrovirus genome, a recombinant Euproctis pseudoconspersa nucleopolyhedrovirus genome, a recombinant Helicoverpa armigera nucleopolyhedrovirus genome, a recombinant Hemileuca species nucleopolyhedrovirus genome, a recombinant Hyphantria cunea nucleopolyhedrovirus genome, a recombinant Hyposidra talaca nucleopolyhedrovirus genome, a recombinant Lambdina fiscellaria nucleopolyhedrovirus genome, a recombinant Leucania separata nucleopolyhedrovirus genome, a recombinant Lonomia obliqua nucleopolyhedrovirus genome, a recombinant Lymantria dispar multiple nucleopolyhedrovirus genome, a recombinant Lymantria xylina nucleopolyhedrovirus genome, a recombinant Mamestra brassicae multiple nucleopolyhedrovirus genome, a recombinant Mamestra configurata nucleopolyhedrovirus A genome, a recombinant Mamestra configurata nucleopolyhedrovirus B genome, a recombinant Maruca vitrata nucleopolyhedrovirus genome, a recombinant Mythimna unipuncta nucleopolyhedrovirus A genome, a recombinant Mythimna unipuncta nucleopolyhedrovirus B genome, a recombinant Operophtera brumata nucleopolyhedrovirus genome, a recombinant Orgyia leucostigma nucleopolyhedrovirus genome, a recombinant Orgyia pseudotsugata multiple nucleopolyhedrovirus genome, a recombinant Oxyplax ochracea nucleopolyhedrovirus genome, a recombinant Peridroma saucia nucleopolyhedrovirus genome, a recombinant Perigonia lusca nucleopolyhedrovirus genome, a recombinant Spodoptera eridania nucleopolyhedrovirus genome, a recombinant Spodoptera exempta nucleopolyhedrovirus genome, a recombinant Spodoptera exigua multiple nucleopolyhedrovirus A genome, a recombinant Spodoptera exigua multiple nucleopolyhedrovirus B genome, a recombinant Spodoptera frugiperda multiple nucleopolyhedrovirus genome, a recombinant Spodoptera littoralis nucleopolyhedrovirus genome, a recombinant Spodoptera litura nucleopolyhedrovirus genome, a recombinant Sucra jujuba nucleopolyhedrovirus genome, a recombinant Thysanoplusia orichalcea nucleopolyhedrovirus genome, a recombinant Trichoplusia ni single nucleopolyhedrovirus genome, a recombinant Urbanus proteus nucleopolyhedrovirus genome, a recombinant Wiseana signata nucleopolyhedrovirus genome, and any combinations or derivatives thereof.

In some embodiments, the recombinant betabaculovirus genome may be a recombinant Adoxophyes orana granulovirus genome, a recombinant Agrotis segetum granulovirus genome, a recombinant Artogeia rapae granulovirus genome, a recombinant Choristoneura fumiferana granulovirus genome, a recombinant Clostera anachoreta granulovirus genome, a recombinant Clostera anastomosis granulovirus A genome, a recombinant Clostera anastomosis granulovirus B genome, a recombinant Cnaphalocrocis medinalis granulovirus genome, a recombinant Cryptophlebia leucotreta granulovirus genome, a recombinant Cydia pomonella granulovirus genome, a recombinant Diatraea saccharalis granulovirus genome, a recombinant Epinotia aporema granulovirus genome, a recombinant Erinnyis ello granulovirus genome, a recombinant Harrisina brillians granulovirus genome, a recombinant Helicoverpa armigera granulovirus genome, a recombinant Lacanobia oleracea granulovirus genome, a recombinant Mocis latipes granulovirus genome, a recombinant Mythimna unipuncta granulovirus A genome, a recombinant Mythimna unipuncta granulovirus B genome, a recombinant Phthorimaea operculella granulovirus genome, a recombinant Plodia interpunctella granulovirus genome, a recombinant Plutella xylostella granulovirus genome, a recombinant Spodoptera frugiperda granulovirus genome, a recombinant Spodoptera litura granulovirus genome, a recombinant Trichoplusia ni granulovirus genome, a recombinant Xestia c-nigrum granulovirus genome, and any combinations or derivatives thereof.

In some embodiments, the recombinant deltabaculovirus genome may be a recombinant Culex nigripalpus nucleopolyhedrovirus genome.

In some embodiments, the recombinant gammabaculovirus genome may be a recombinant Neodiprion lecontei nucleopolyhedrovirus genome, a recombinant Neodiprion sertifer nucleopolyhedrovirus genome, and any combinations or derivatives thereof.

In some embodiments, one or more of the recombinant nucleic acids described herein are a recombinant geminivirus genome. The recombinant geminivirus genome may be a recombinant genome from any member of the Geminiviridae family of viruses known in the art, including, for example, a recombinant Becurtovirus genome, a recombinant Begomovirus genome, a recombinant Capulavirus genome, a recombinant Citlodavirus genome, a recombinant Curtovirus genome, a recombinant Eragrovirus genome, a recombinant Grablovirus genome, a recombinant Maldovirus genome, a recombinant Mastrevirus genome, a recombinant Mulcrilevirus genome, a recombinant Opunvirus genome, a recombinant Topilevirus genome, a recombinant Topocuvirus genome, a recombinant Turncurtovirus genome, and any combinations or derivatives thereof. In some embodiments, the recombinant geminivirus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) geminivirus genes.

In some embodiments, one or more of the recombinant nucleic acids described herein are a recombinant caulimovirus genome. The recombinant caulimovirus genome may be a recombinant genome from any member of the Caulimovirus family of viruses known in the art, including, for example, a recombinant Angelica bushy stunt virus genome, a recombinant Atractylodes mild mottle virus genome, a recombinant Carnation etched ring virus genome, a recombinant Cauliflower mosaic virus genome, a recombinant Dahlia mosaic virus genome, a recombinant Figwort mosaic virus genome, a recombinant Horseradish latent virus genome, a recombinant Lamium leaf distortion virus genome, a recombinant Mirabilis mosaic virus genome, a recombinant Soybean Putnam virus genome, a recombinant Strawberry vein banding virus genome, a recombinant Thistle mottle virus genome, and any combinations or derivatives thereof. In some embodiments, the recombinant caulimovirus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) caulimovirus genes.

In some embodiments, one or more of the recombinant nucleic acids described herein are a recombinant anellovirus genome. The recombinant anellovirus genome may be a recombinant genome from any member of the anellovirus family of viruses known in the art, including, for example, a recombinant Aleptorquevirus genome, a recombinant Alphatorquevirus genome, a recombinant Betatorquevirus genome, a recombinant Chitorquevirus genome, a recombinant Dalettorquevirus genome, a recombinant Deltatorquevirus genome, a recombinant Epsilontorquevirus genome, a recombinant Etatorquevirus genome, a recombinant Gammatorquevirus genome, a recombinant Gimeltorquevirus genome, a recombinant Gyrovirus genome, a recombinant Hetorquevirus genome, a recombinant lotatorquevirus genome, a recombinant Kappatorquevirus genome, a recombinant Lambdatorquevirus genome, a recombinant Mutorquevirus genome, a recombinant Nutorquevirus genome, a recombinant Omegatorquevirus genome, a recombinant Omicrontorquevirus genome, a recombinant Pitorquevirus genome, a recombinant Psitorquevirus genome, a recombinant Rhotorquevirus genome, a recombinant Sigmatorquevirus genome, a recombinant Tettorquevirus genome, a recombinant Thetatorquevirus genome, a recombinant Upsilontorquevirus genome, a recombinant Wawtorquevirus genome, a recombinant Xitorquevirus genome, a recombinant Zayintorquevirus genome, a recombinant Zetatorquevirus genome, and any combinations or derivatives thereof. In some embodiments, the recombinant anellovirus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) anellovirus genes.

### V. GENE DELIVERY VEHICLE

### Viral Vectors or Viral Gene Delivery Vehicle

Certain aspects of the present disclosure relate to viral vectors or viral gene delivery vehicles comprising any of the polynucleotides and/or recombinant nucleic acids described herein. In some embodiments, the virus is capable of infecting one or more target cells of a subject (e.g., a human). In some embodiments, the virus is suitable for delivering the polynucleotides and/or recombinant nucleic acids into one or more target cells of a subject (e.g., a human). In some embodiments, the one or more target cells are human cells. In some embodiments, the one or more target cells are one or more cancerous cells. In some embodiments, the one or more target cells are one or more non-cancerous (e.g., healthy) cells. In some embodiments, the one or more target cells are one or more airway epithelial cells. In some embodiments, the one or more target cells are one or more cells of the respiratory tract (e.g., airway epithelial cells (such as goblet cells, ciliated cells, Clara cells, neuroendocrine cells, basal cells, intermediate or parabasal cells, Serous cells, brush cells, oncocytes, non-ciliated columnar cells, and/or metaplastic cells); alveolar cells (such as type 1 pneumocytes, type 2 pneumocytes, and/or cuboidal non-ciliated cells); salivary gland cells in bronchi (such as Serous cells, mucous cells, and/or ductal cells); etc.). In some embodiments, the one or more target cells are one or more cells of the lung.

Any suitable virus known in the art may be used, including, for example, adenovirus, adeno-associated virus, retrovirus, lentivirus, sendai virus, papillomavirus, herpes virus (e.g., a herpes simplex virus), vaccinia virus (e.g., a poxvirus), a bacteriophage, an alphavirus, a picornavirus, an iridovirus, a Newcastle virus, a baculovirus, a geminivirus, a caulimovirus, anellovirus, and/or any hybrid or derivative viruses thereof. In some embodiments, the virus is attenuated. In some embodiments, the virus is replication competent. In some embodiments, the virus is replication defective. In some embodiments, the virus is not oncolytic. In some embodiments, the virus has been modified to alter its tissue tropism relative to the tissue tropism of a corresponding unmodified, wild-type virus. In some embodiments, the virus has reduced cytotoxicity (e.g., in a target cell) as compared to a corresponding wild-type virus. Methods of producing a virus comprising polynucleotides and/or recombinant nucleic acids are well known to one of ordinary skill in the art.

In some embodiments, the viral gene delivery vehicle is an adenovirus. The adenovirus may be from any member of the Adenoviridae family of viruses known in the art, including, for example, a recombinant Atadenovirus, a recombinant Aviadenovirus, a recombinant Ichtadenovirus, a recombinant Mastadenovirus, a recombinant Siadenovirus, a recombinant Testadenovirus, and any combinations or derivatives thereof. In some embodiments, the adenovirus is replication competent. In some embodiments, the adenovirus is replication defective or replication incompetent. In some embodiments, the adenovirus is not oncolytic.

In some embodiments, the viral gene delivery vehicle is a retrovirus. The retrovirus may be from any member of the Retroviridae family of viruses known in the art, including, for example, a recombinant Alpharetrovirus, a recombinant Betaretrovirus, a recombinant Gammaretrovirus, a recombinant Deltaretrovirus, a recombinant Epsilonretrovirus, a recombinant Lentivirus, a recombinant Bovispumavirus, a recombinant Equispumavirus, a recombinant Felispumavirus, a recombinant Prosimiispumavirus, a recombinant Simiispumavirus, and any combinations or derivatives thereof. In some embodiments, the retrovirus is replication competent. In some embodiments, the retrovirus is replication defective or replication incompetent. In some embodiments, the retrovirus is not oncolytic.

In some embodiments, the viral gene delivery vehicle is an adeno-associated virus (AAV). The AAV may be from any member of the Dependoparvovirus family of viruses known in the art, including, for example, a recombinant Adeno-associated dependoparvovirus A, a recombinant Adeno-associated dependoparvovirus B, and any combinations or derivatives thereof. In some embodiments, the AAV is replication competent. In some embodiments, the AAV is replication defective or replication incompetent. In some embodiments, the AAV is not oncolytic.

In some embodiments, the AAV may be from any serotype known in the art, including, for example, serotype 1, serotype 2, serotype 3, serotype 4, serotype 5, serotype 6, serotype 7, serotype 8, AAV serotype 9, serotype 10, serotype 11, and any combinations or derivatives thereof.

In some embodiments, the viral gene delivery vehicle is a member of the Herpesviridae family of DNA viruses, including, for example, a herpes simplex virus, a varicella zoster virus, a human cytomegalovirus, a herpesvirus 6A, a herpesvirus 6B, a herpesvirus 7, an Epstein-Barr virus, and a Kaposi's sarcoma-associated herpesvirus, and any combinations or derivatives thereof. In some embodiments, the Herpesviridae virus is replication competent. In some embodiments, the Herpesviridae virus is replication defective or replication incompetent. In some embodiments, the Herpesviridae virus is not oncolytic.

In some embodiments, the herpesvirus is a herpes simplex virus. In some embodiments, the herpes simplex virus is an HSV-1, an HSV-2, or any combinations or derivatives thereof. In some embodiments, the herpes simplex virus is an HSV-1 virus.

In some embodiments, the viral gene delivery vehicle is a poxvirus. The poxvirus may be from any member of the Poxviridae family of viruses known in the art, including, for example, a recombinant smallpox virus, a recombinant vaccinia virus, a recombinant cowpox virus, a recombinant monkeypox virus, a recombinant orf virus, a recombinant pseudocowpox virus, a recombinant bovine papular stomatitis virus, a recombinant tanapox virus, a recombinant yaba monkey tumor virus, a recombinant molluscum contagiosum virus, and any combinations or derivatives thereof. In some embodiments, the poxvirus is replication competent. In some embodiments, the poxvirus is replication defective or replication incompetent. In some embodiments, the poxvirus is not oncolytic.

In some embodiments, the viral gene delivery vehicle is a bacteriophage. The bacteriophage may be any bacteriophage known in the art, including, for example, a recombinant 186 phage, a recombinant Escherichia virus Lambda, a recombinant Pseudomonas virus phi6, a recombinant Bacillus virus φ29, a recombinant Escherichia virus φX174, a recombinant Bacteriophage φCb5, a recombinant Escherichia virus G4, a recombinant Escherichia virus M13, a recombinant Emesvirus zinderi, a recombinant N4 phage, a recombinant Escherichia virus P1, a recombinant Escherichia virus P2, a recombinant Enterobacteria phage P4, a recombinant R17 phage, a recombinant Enterobacteria phage T2, a recombinant Escherichia virus T4, a recombinant Escherichia virus T7, a recombinant Bacteriophage T12, and any combinations or derivatives thereof. In some embodiments, the bacteriophage is replication competent. In some embodiments, the bacteriophage is replication defective or replication incompetent. In some embodiments, the bacteriophage is not oncolytic.

In some embodiments, the viral gene delivery vehicle is an alphavirus. The alphavirus may be from any member of the Alphavirus family of viruses known in the art, including, for example, a recombinant Aura virus, a recombinant Barmah Forest virus, a recombinant Bebaru virus, a recombinant Caaingua virus, a recombinant Cabassou virus, a recombinant Chikungunya virus, a recombinant Eastern equine encephalitis virus, a recombinant Eilat virus, a recombinant Everglades virus, a recombinant Fort Morgan virus, a recombinant Getah virus, a recombinant Highlands J virus, a recombinant Madariaga virus, a recombinant Mayaro virus, a recombinant Middelburg virus, a recombinant Mosso das Pedras virus, a recombinant Mucambo virus, a recombinant Ndumu virus, a recombinant O'nyong'nyong virus, a recombinant Pixuna virus, a recombinant Rio Negro virus, a recombinant Ross River virus, a recombinant Salmon pancreas disease virus, a recombinant Semliki Forest virus, a recombinant Sindbis virus, a recombinant Southern elephant seal virus, a recombinant Tonate virus, a recombinant Trocara virus, a recombinant Una virus, a recombinant Venezuelan equine encephalitis virus, a recombinant Western equine encephalitis virus, a recombinant Whataroa virus, and any combinations or derivatives thereof. In some embodiments, the alphavirus is replication competent. In some embodiments, the alphavirus is replication defective or replication incompetent. In some embodiments, the alphavirus is not oncolytic.

In some embodiments, the viral gene delivery vehicle is a picornavirus. The picornavirus may be from any member of the Picornaviridae family of viruses known in the art, including, for example, a recombinant Aalivirus, a recombinant Ailurivirus, a recombinant Ampivirus, a recombinant Anativirus, a recombinant Aphthovirus, a recombinant Aquamavirus, a recombinant Avihepatovirus, a recombinant Avisivirus, a recombinant Boosepivirus, a recombinant Bopivirus, a recombinant Caecilivirus, a recombinant Cardiovirus, a recombinant Cosavirus, a recombinant Crahelivirus, a recombinant Crohivirus, a recombinant Danipivirus, a recombinant Dicipivirus, a recombinant Diresapivirus, a recombinant Enterovirus, a recombinant Erbovirus, a recombinant Felipivirus, a recombinant Fipivirus, a recombinant Gallivirus, a recombinant Gruhelivirus, a recombinant Grusopivirus, a recombinant Harkavirus, a recombinant Hemipivirus, a recombinant Hepatovirus, a recombinant Hunnivirus, a recombinant Kobuvirus, a recombinant Kunsagivirus, a recombinant Limnipivirus, a recombinant Livupivirus, a recombinant Ludopivirus, a recombinant Malagasivirus, a recombinant Marsupivirus, a recombinant Megrivirus, a recombinant Mischivirus, a recombinant Mosavirus, a recombinant Mupivirus, a recombinant Myrropivirus, a recombinant Orivirus, a recombinant Oscivirus, a recombinant Parabovirus, a recombinant Parechovirus, a recombinant Pasivirus, a recombinant Passerivirus, a recombinant Pemapivirus, a recombinant Poecivirus, a recombinant Potamipivirus, a recombinant Pygoscepivirus, a recombinant Rabovirus, a recombinant Rafivirus, a recombinant Rajidapivirus, a recombinant Rohelivirus, a recombinant Rosavirus, a recombinant Sakobuvirus, a recombinant Salivirus, a recombinant Sapelovirus, a recombinant Senecavirus, a recombinant Shanbavirus, a recombinant Sicinivirus, a recombinant Symapivirus, a recombinant Teschovirus, a recombinant Torchivirus, a recombinant Tottorivirus, a recombinant Tremovirus, a recombinant Tropivirus, and any combinations or derivatives thereof. In some embodiments, the picornavirus is replication competent. In some embodiments, the picornavirus is replication defective or replication incompetent. In some embodiments, the picornavirus is not oncolytic.

In some embodiments, the viral gene delivery vehicle is an iridovirus. The iridovirus may be from any member of the Iridovirus family of viruses known in the art, including, for example, a recombinant Invertebrate iridescent virus 6 (IIV-6), a recombinant Invertebrate iridescent virus 31 (IIV-31), and any combinations or derivatives thereof. In some embodiments, the iridovirus is replication competent. In some embodiments, the iridovirus is replication defective or replication incompetent. In some embodiments, the iridovirus is not oncolytic.

In some embodiments, the viral gene delivery vehicle is a Newcastle disease virus. In some embodiments, the Newcastle disease virus is replication competent. In some embodiments, the Newcastle disease virus is replication defective or replication incompetent. In some embodiments, the Newcastle disease virus is not oncolytic.

In some embodiments, the viral gene delivery vehicle is a baculovirus. The baculovirus may be from any member of the Baculoviridae family of viruses known in the art, including, for example, a recombinant alphabaculovirus, a recombinant betabaculovirus, a recombinant deltabaculovirus, a recombinant gammabaculovirus, and any combinations or derivatives thereof. In some embodiments, the baculovirus is replication competent. In some embodiments, the baculovirus is replication defective or replication incompetent. In some embodiments, the baculovirus is not oncolytic.

In some embodiments, the viral gene delivery vehicle is a gemininvirus. The geminivirus may be from any member of the Geminiviridae family of viruses known in the art, including, for example, a recombinant Becurtovirus, a recombinant Begomovirus, a recombinant Capulavirus, a recombinant Citlodavirus, a recombinant Curtovirus, a recombinant Eragrovirus, a recombinant Grablovirus, a recombinant Maldovirus, a recombinant Mastrevirus, a recombinant Mulcrilevirus, a recombinant Opunvirus, a recombinant Topilevirus, a recombinant Topocuvirus, a recombinant Turncurtovirus, and any combinations or derivatives thereof. In some embodiments, the gemininvirus is replication competent. In some embodiments, the gemininvirus is replication defective or replication incompetent. In some embodiments, the geminivirus is not oncolytic.

In some embodiments, the viral gene delivery vehicle is a caulimovirus. The caulimovirus may be from any member of the Caulimovirus family of viruses known in the art, including, for example, a recombinant Angelica bushy stunt virus, a recombinant Atractylodes mild mottle virus, a recombinant Carnation etched ring virus, a recombinant Cauliflower mosaic virus, a recombinant Dahlia mosaic virus, a recombinant Figwort mosaic virus, a recombinant Horseradish latent virus, a recombinant Lamium leaf distortion virus, a recombinant Mirabilis mosaic virus, a recombinant Soybean Putnam virus, a recombinant Strawberry vein banding virus, a recombinant Thistle mottle virus, and any combinations or derivatives thereof. In some embodiments, the caulimovirus is replication competent. In some embodiments, the caulimovirus is replication defective or replication incompetent. In some embodiments, the caulimovirus is not oncolytic.

In some embodiments, the viral gene delivery vehicle is an anellovirus. The anellovirus may be from any member of the Anelloviridae family of viruses known in the art, including, for example, a recombinant Aleptorquevirus, a recombinant Alphatorquevirus, a recombinant Betatorquevirus, a recombinant Chitorquevirus, a recombinant Dalettorquevirus, a recombinant Deltatorquevirus, a recombinant Epsilontorquevirus, a recombinant Etatorquevirus, a recombinant Gammatorquevirus, a recombinant Gimeltorquevirus, a recombinant Gyrovirus, a recombinant Hetorquevirus, a recombinant lotatorquevirus, a recombinant Kappatorquevirus, a recombinant Lambdatorquevirus, a recombinant Mutorquevirus, a recombinant Nutorquevirus, a recombinant Omegatorquevirus, a recombinant Omicrontorquevirus, a recombinant Pitorquevirus, a recombinant Psitorquevirus, a recombinant Rhotorquevirus, a recombinant Sigmatorquevirus, a recombinant Tettorquevirus, a recombinant Thetatorquevirus, a recombinant Upsilontorquevirus, a recombinant Wawtorquevirus, a recombinant Xitorquevirus, a recombinant Zayintorquevirus, a recombinant Zetatorquevirus, and any combinations or derivatives thereof. In some embodiments, the anellovirus is replication competent. In some embodiments, the anellovirus is replication defective or replication incompetent. In some embodiments, the anellovirus is not oncolytic.

In some embodiments, provided herein are recombinant viruses, which may or may not be pseudotyped, that produce IL-2 and IL-12 polypeptides for the treatment of cancer including solid tumors (e.g., advanced solid tumors) and hematologic malignancies. In some embodiments, the therapeutic polypeptides produced by the recombinant viruses described herein mediate or enhance an anti-tumor effect, such as by effector-cell mediated lysis of tumor cells.

### Non-Viral Vectors or Non-Viral Gene Delivery Vehicles

Certain aspects of the present disclosure relate to non-viral vectors or non-viral gene delivery vehicles comprising any of the polynucleotides and/or recombinant nucleic acids described herein. In some embodiments, the non-viral vector or non-viral gene delivery vehicle is capable of delivering the polynucleotides and/or recombinant nucleic acids to one or more target cells of a subject (e.g., a human). In some embodiments, the non-viral vector or non-viral gene delivery vehicle is suitable for delivering the polynucleotides and/or recombinant nucleic acids into one or more target cells of a subject (e.g., a human). In some embodiments, the one or more target cells are human cells. In some embodiments, the one or more target cells are one or more cancerous cells. In some embodiments, the one or more target cells are one or more non-cancerous (e.g., healthy) cells. In some embodiments, the one or more target cells are one or more airway epithelial cells. In some embodiments, the one or more target cells are one or more cells of the respiratory tract (e.g., airway epithelial cells (such as goblet cells, ciliated cells, Clara cells, neuroendocrine cells, basal cells, intermediate or parabasal cells, Serous cells, brush cells, oncocytes, non-ciliated columnar cells, and/or metaplastic cells); alveolar cells (such as type 1 pneumocytes, type 2 pneumocytes, and/or cuboidal non-ciliated cells); salivary gland cells in bronchi (such as Serous cells, mucous cells, and/or ductal cells); etc.). In some embodiments, the one or more target cells are one or more cells of the lung.

Any non-viral vector or non-viral gene delivery vehicle known in the art may be used, including, for example, a chemical compound, a bacterium, a mammalian cell, or a physical delivery system and/or any hybrid or derivative thereof. Methods of producing a non-viral vector or non-viral gene delivery vehicle comprising polynucleotides and/or recombinant nucleic acids are well known to one of ordinary skill in the art.

In some embodiments, the non-viral vector or non-viral gene delivery vehicle is a chemical compound. In some embodiments, the chemical compound may be a polymer compound, a lipid compound, an inorganic compound, and any combinations or derivatives thereof.

In some embodiments, the polymer compound may be a natural polymer, a synthetic polymer, a biopolymer, a biodegradable polymer, a cationic polymer, a protein polymer, a polysaccharide polymer, and any combinations or derivatives thereof.

In some embodiments, the polymer compound may be poly-L-lactide (PLA), chitosan, pectin, polyethylenimine (PEI), poly-L-lysine (PLL), poly 2-N-dimethylaminoethyl methacrylate (PDMAEMA), polyaminoamine, polyaminoesters, polyamidoamine, poly-vinylimidazole (PVI), Poly-beta-amino ester (PBAE), gelatin, albumin, chitosan, beta-cyclodextrin, dextran, hyaluronic acid, polyester, alpha-Pyrrolidinohexiophenone (PHP), Poly-2-acrylamidoglycolic acid (PAGA), Poly-delta-valerolactone (PVL), polyphenyl ethers (PPE), Poly-beta-amino-ester (PBAE), poly-amine-co-ester (PACE), poly-lactic-co-glycolic acid (PLGA), polycarbonates, polyurethanes, micelles, cationic emulsion, exosome, Reb Blood Cell membrane, DNA-protein complexes, plasmid DNA, naked DNA, pBR322, pUC, and any combinations or derivatives thereof.

In some embodiments, the lipid compound may be a nanoparticle (e.g., as disclosed in U.S. Patent Application Publication No. 2010/0015232, the contents of which are hereby incorporated by reference in its entirety), a lipid nanoparticle (LNP; e.g., as disclosed in U.S. Pat. Nos. 10,898,574, 10,702,600, and 10,933,127, the contents of which are hereby incorporated by reference in its entirety), a cell targeted lipid nanoparticle (ctLNP; e.g., as disclosed in U.S. Patent Application Publication Nos. 2021/0059953, 2020/0283794, and/or 2021/0108228 the contents of which are hereby incorporated by reference in its entirety), a liposome, a cationic liposome, a solid lipid nanoparticle, a lipid emulsion, a lipidoid, a cytofectin, a lipid emulsion, a surfactant, a gemini surfactant, and any combinations or derivatives thereof.

In some embodiments, the inorganic compound may be a nanocarrier, a DNA nanclew, a gold nanoparticle, a carbon nanotube, a graphene, a quantum dot, an up-conversion nanoparticle, a silica nanoparticle, an iron oxide, a ferritin, and any combinations or derivatives thereof.

In some embodiments, the non-viral vector or non-viral gene delivery vehicle is a physical delivery system. In some embodiments, the physical delivery system may be electroporation, gene gun, jet gun, ultrasound, nucleofection, hydrodynamic gene delivery, needle injection, microinjection, ballistic DNA injection, sonoporation, photoporation, laser pulse, magnetofection, magnetoporation, magnetic particles, hydroporation, and any combinations or derivatives thereof.

### VI. PHARMACEUTICAL COMPOSITIONS AND FORMULATIONS

Certain aspects of the present disclosure relate to pharmaceutical compositions or formulations comprising any of the polynucleotides, recombinant nucleic acids, and/or gene delivery vehicles comprising the polynucleotide(s) and/or recombinant nucleic acids, and a pharmaceutically acceptable excipient or carrier.

Pharmaceutical compositions and formulations can be prepared by mixing the active ingredient(s) (such as a polynucleotide, a recombinant nucleic acid comprising the polynucleotide, a gene delivery vehicle comprising the polynucleotide and/or recombinant nucleic acid) having the desired degree of purity with one or more pharmaceutically acceptable carriers or excipients. Pharmaceutically acceptable carriers or excipients are generally nontoxic to recipients at the dosages and concentrations employed, and may include, but are not limited to: buffers (such as phosphate, citrate, acetate, and other organic acids); antioxidants (such as ascorbic acid and methionine); preservatives (such as octadecyldimethylbenzyl ammonium chloride, benzalkonium chloride, benzethonium chloride, phenol, butyl or benzyl alcohol, alkyl parabens, catechol, resorcinol, cyclohexanol, 3-pentanol, and m-cresol); amino acids (such as glycine, glutamine, asparagine, histidine, arginine, or lysine); low molecular weight (less than about 10 residues) polypeptides; proteins (such as serum albumin, gelatin, or immunoglobulins); polyols (such as glycerol, e.g., formulations including 1%, 2%, 3%, 4%, 5%, 6%, 7%, 8%, 9%, 10%, 11%, 12%, 13%, 14%, 15%, etc. glycerol); hydrophilic polymers (such as polyvinylpyrrolidone); monosaccharides, disaccharides, and other carbohydrates (including glucose, mannose, or dextrins); chelating agents (such as EDTA); sugars (such as sucrose, mannitol, trehalose, or sorbitol); salt-forming counter-ions (such as sodium); metal complexes (such as Zn-protein complexes); and/or non-ionic surfactants (such as polyethylene glycol (PEG)). A thorough discussion of pharmaceutically acceptable carriers is available in REMINGTON'S PHARMACEUTICAL SCIENCES (Mack Pub. Co., N.J. 1991).

In some embodiments, the pharmaceutical composition or formulation comprises one or more lipid (e.g., cationic lipid) carriers. In some embodiments, the pharmaceutical composition or formulation comprises one or more nanoparticle carriers. A variety of molecules (e.g., proteins, peptides, recombinant nucleic acids, etc.) can be efficiently encapsulated in nanoparticles using processes well known in the art. In some embodiments, a molecule “encapsulated” in a nanoparticle may refer to a molecule that is contained within the nanoparticle or attached to and/or associated with the surface of the nanoparticle, or any combination thereof. Nanoparticles for use in the compositions or formulations described herein may be any type of biocompatible nanoparticle known in the art, including, for example, nanoparticles comprising poly(lactic acid), poly(glycolic acid), PLGA, PLA, PGA, and any combinations thereof (see e.g., Vauthier et al. Adv Drug Del Rev. (2003) 55:519-48; US 2007/0148074; US 2007/0092575; US 2006/0246139; U.S. Pat. Nos. 5,753,234; 7,081,483; and US 2008/0260851, all of which are incorporated by reference herein in their entirety).

In some embodiments, the pharmaceutically acceptable carrier or excipient may be adapted for or suitable for any administration route known in the art, including, for example, intratumoral, intravenous, intramuscular, subcutaneous, cutaneous, oral, intranasal, intratracheal, sublingual, buccal, topical, transdermal, intradermal, intraperitoneal, intraorbital, intravitreal, subretinal, transmucosal, intraarticular, by implantation, by inhalation, intrathecal, intraventricular, intracranial, and/or intranasal administration. In some embodiments, the pharmaceutically acceptable carrier or excipient is adapted for or suitable for oral, intranasal, intratracheal, and/or inhaled administration. In some embodiments, the pharmaceutically acceptable carrier or excipient is adapted for or suitable for intranasal and/or inhaled administration. In some embodiments, the pharmaceutically acceptable carrier or excipient is adapted for or suitable for inhaled administration. In some embodiments, the pharmaceutically acceptable carrier or excipient is adapted for or suitable for intratumoral administration.

In some embodiments, the pharmaceutical composition or formulation is adapted for or suitable for any administration route known in the art, including, for example, intratumoral, intravenous, intramuscular, subcutaneous, cutaneous, oral, intranasal, intratracheal, sublingual, buccal, topical, transdermal, intradermal, intraperitoneal, intraorbital, intravitreal, subretinal, transmucosal, intraarticular, by implantation, by inhalation, intrathecal, intraventricular, intracranial, or intranasal administration. In some embodiments, the pharmaceutical composition or formulation is adapted for or suitable for oral, intranasal, intratracheal, and/or inhaled administration. In some embodiments, the pharmaceutical composition or formulation is adapted for or suitable for intranasal and/or inhaled administration. In some embodiments, the pharmaceutical composition or formulation is adapted for or suitable for inhaled administration. In some embodiments, the pharmaceutical composition or formulation is adapted for or suitable for intratumoral administration.

In some embodiments, the pharmaceutical composition or formulation further comprises one or more additional components. Examples of additional components may include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); wetting agents (e.g., sodium lauryl sulphate, etc.); salt solutions; alcohols; polyethylene glycols; gelatin; lactose; amylase; magnesium stearate; talc; silicic acid; viscous paraffin; hydroxymethylcellulose; polyvinylpyrrolidone; sweetenings; flavorings; perfuming agents; colorants; moisturizers; sunscreens; antibacterial agents; agents able to stabilize polynucleotides or prevent their degradation, and the like. In some embodiments, the pharmaceutical composition or formulation comprises a methylcellulose gel (e.g., hydroxypropyl methylcellulose, carboxy methylcellulose, etc.). In some embodiments, the pharmaceutical composition or formulation comprises a phosphate buffer. In some embodiments, the pharmaceutical composition or formulation comprises glycerol (e.g., at about 1%, about 2%, about 3%, about 4%, about 5%, about 6%, about 7%, about 8%, about 9%, about 10%, about 11%, about 12%, about 13%, about 14%, about 15%, etc.). In some embodiments, the pharmaceutical composition or formulation comprises a phosphate buffer and glycerol.

Pharmaceutical compositions and formulations to be used for in vivo administration are generally sterile. Sterility may be readily accomplished, e.g., by filtration through sterile filtration membranes.

In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used to deliver one or more polynucleotides encoding IL-2 and IL-12 polypeptides into one or more cells of a subject (e.g., one or more cells of the respiratory tract of the subject). In some embodiments, the subject suffers from cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of carcinoma. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of lymphoma. In some embodiments any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of blastoma. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of sarcoma. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of a neuroendocrine tumor. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of mesothelioma. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of schwannoma. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of meningioma. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of adenocarcinoma. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of melanoma. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of leukemia. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of lymphoid malignancy.

In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used to deliver one or more polynucleotides encoding an IL-2 and IL-12 polypeptide into one or more cells of a subject (e.g., one or more cells of the respiratory tract of the subject). In some embodiments, the subject suffers from cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of a solid tumor. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of a hematologic cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of bladder cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of brain cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of breast cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of colon cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of gastric cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of glioma. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of head cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of leukemia. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of liver cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of lung cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of lymphoma. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of myeloma. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of neck cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of ovarian cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of melanoma. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of pancreatic cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of renal cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of salivary cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of skin cancer (e.g., melanoma, basal cell carcinoma, squamous cell carcinoma, actinic keratosis, an atypical mole, and/or Merkel cell carcinoma). In some embodiments any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of stomach cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of thymic epithelial cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of thyroid cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of osteosarcoma. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of leptomeningeal cancer.

In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used to deliver one or more polynucleotides encoding an IL-2 and IL-12 polypeptide into one or more cells of a subject (e.g., one or more cells of the respiratory tract of the subject). In some embodiments, the subject suffers from lung cancer. Lung cancers are often divided into the broad categories of small-cell lung cancer (SCLC), also called oat cell cancer, and non-small-cell lung cancer (NSCLC). NSCLC is further divided into three major types, squamous, cell carcinoma (SCC), adenocarcinoma and large cell carcinomas. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of small-cell lung cancer. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of adenocarcinoma of the lung. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of squamous carcinoma of the lung. In some embodiments, any of the polynucleotides, recombinant nucleic acids comprising the polynucleotides, gene delivery vehicles comprising the polynucleotides and/or recombinant nucleic acids, and/or pharmaceutical compositions or formulations described herein may be used in the treatment of non-small cell lung cancer.

### VII. METHODS

Certain aspects of the present disclosure relate to a method of delivering one or more polynucleotides to one or more cells of a subject (e.g., one or more cancerous cells, one or more non-cancerous (e.g., healthy) cells, one or more cells of the respiratory tract, such as airway epithelial cells (goblet cells, ciliated cells, Clara cells, neuroendocrine cells, basal cells, intermediate or parabasal cells, Serous cells, brush cells, oncocytes, non-ciliated columnar cells, and/or metaplastic cells); alveolar cells (type 1 pneumocytes, type 2 pneumocytes, and/or cuboidal non-ciliated cells); salivary gland cells in bronchi (Serous cells, mucous cells, and/or ductal cells); etc.) comprising administering to the subject any of the polynucleotides, recombinant nucleic acids comprising the polynucleotides, gene delivery vehicles comprising the polynucleotides and/or recombinant nucleic acids, and/or pharmaceutical compositions or formulations described herein. In some embodiments, the polynucleotides, recombinant nucleic acids comprising the polynucleotides, gene delivery vehicles comprising the polynucleotides and/or recombinant nucleic acids, and/or pharmaceutical compositions or formulations are administered topically, transdermally, subcutaneously, epicutaneously, intradermally, orally, sublingually, buccally, rectally, vaginally, intravenously, intraarterially, intramuscularly, intraosseously, intracardially, intraperitoneally, transmucosally, intravitreally, subretinally, suprachoroidally, intracranially, intrathecally, intraventricularly, intraarticularly, peri-articularly, intratumorally, locally, or via inhalation to the subject. In some embodiments, the polynucleotides, recombinant nucleic acids comprising the polynucleotides, gene delivery vehicles comprising the polynucleotides and/or recombinant nucleic acids, and/or pharmaceutical compositions or formulations are administered orally, intranasally, intratracheally, or via inhalation to the subject. In some embodiments, the polynucleotides, recombinant nucleic acids comprising the polynucleotides, gene delivery vehicles comprising the polynucleotides and/or recombinant nucleic acids, and/or pharmaceutical compositions or formulations are administered intranasally or via inhalation to the subject. In some embodiments, the polynucleotides, recombinant nucleic acids comprising the polynucleotides, gene delivery vehicles comprising the polynucleotides and/or recombinant nucleic acids, and/or pharmaceutical compositions or formulations are administered via inhalation to the subject. In some embodiments, the polynucleotides, recombinant nucleic acids comprising the polynucleotides, gene delivery vehicles comprising the polynucleotides and/or recombinant nucleic acids, and/or pharmaceutical compositions or formulations are administered using a dry powder inhaler, a pressurized metered dose inhaler, a soft mist inhaler, a nebulizer, or an electrohydrodynamic aerosol device. In some embodiments, the polynucleotides, recombinant nucleic acids comprising the polynucleotides, gene delivery vehicles comprising the polynucleotides and/or recombinant nucleic acids, and/or pharmaceutical compositions or formulations are administered using a nebulizer. In some embodiments, the nebulizer is a vibrating mesh nebulizer. In some embodiments, the polynucleotides, recombinant nucleic acids comprising the polynucleotides, gene delivery vehicles comprising the polynucleotides and/or recombinant nucleic acids, and/or pharmaceutical compositions or formulations intratumorally.

In some embodiments, the subject is a human. In some embodiments, the subject suffers from a cancer. In some embodiments, the cancer is selected from acute myeloid leukemia (LAML or AML), acute lymphoblastic leukemia (ALL), adrenocortical carcinoma (ACC), bladder urothelial cancer (BLCA), brain stem glioma, brain lower grade glioma (LGG), brain tumor, breast cancer (BRCA), bronchial tumors, Burkitt lymphoma, cancer of unknown primary site, carcinoid tumor, carcinoma of unknown primary site, central nervous system atypical teratoid/rhabdoid tumor, central nervous system embryonal tumors, cervical squamous cell carcinoma, endocervical adenocarcinoma (CESC) cancer, childhood cancers, cholangiocarcinoma (CHOL), chordoma, chronic lymphocytic leukemia, chronic myelogenous leukemia, chronic myeloproliferative disorders, colon (adenocarcinoma) cancer (COAD), colorectal cancer, craniopharyngioma, cutaneous T-cell lymphoma, endocrine pancreas islet cell tumors, endometrial cancer, ependymoblastoma, ependymoma, esophageal cancer (ESCA), esthesioneuroblastoma, Ewing sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal cell tumor, gastrointestinal stromal tumor (GIST), gestational trophoblastic tumor, glioblastoma multiforme glioma GBM), hairy cell leukemia, head and neck cancer (HNSD), heart cancer, Hodgkin lymphoma, hypopharyngeal cancer, intraocular melanoma, islet cell tumors, Kaposi sarcoma, kidney cancer, Langerhans cell histiocytosis, laryngeal cancer, leptomeningeal cancer, lip cancer, liver cancer, Lymphoid Neoplasm Diffuse Large B-cell Lymphoma [DLBCL), malignant fibrous histiocytoma bone cancer, medulloblastoma, medulloepithelioma, melanoma, Merkel cell carcinoma, Merkel cell skin carcinoma, mesothelioma (MESO), metastatic squamous neck cancer with occult primary, mouth cancer, multiple endocrine neoplasia syndromes, multiple myeloma, multiple myeloma/plasma cell neoplasm, mycosis fungoides, myelodysplastic syndromes, myeloproliferative neoplasms, nasal cavity cancer, nasopharyngeal cancer, neuroblastoma, Non-Hodgkin lymphoma, nonmelanoma skin cancer, non-small cell lung cancer, oral cancer, oral cavity cancer, oropharyngeal cancer, osteosarcoma, other brain and spinal cord tumors, ovarian cancer, ovarian epithelial cancer, ovarian germ cell tumor, ovarian low malignant potential tumor, pancreatic cancer, papillomatosis, paranasal sinus cancer, parathyroid cancer, pelvic cancer, penile cancer, pharyngeal cancer, pheochromocytoma and paraganglioma (PCPG), pineal parenchymal tumors of intermediate differentiation, pineoblastoma, pituitary tumor, plasma cell neoplasm/multiple myeloma, pleuropulmonary blastoma, primary central nervous system (CNS) lymphoma, primary hepatocellular liver cancer, prostate cancer such as prostate adenocarcinoma (PRAD), rectal cancer, renal cancer, renal cell (kidney) cancer, renal cell cancer, respiratory tract cancer, retinoblastoma, rhabdomyosarcoma, salivary gland cancer, sarcoma (SARC), Sezary syndrome, skin cutaneous melanoma (SKCM), small cell lung cancer, small intestine cancer, soft tissue sarcoma, squamous cell carcinoma, squamous neck cancer, stomach (gastric) cancer, supratentorial primitive neuroectodermal tumors, T-cell lymphoma, testicular cancer testicular germ cell tumors (TGCT), throat cancer, thymic carcinoma, thymoma (THYM), thyroid cancer (THCA), transitional cell cancer, transitional cell cancer of the renal pelvis and ureter, trophoblastic tumor, ureter cancer, urethral cancer, uterine cancer, uterine cancer, uveal melanoma (UVM), vaginal cancer, vulvar cancer, Waldenstrom macroglobulinemia, and/or Wilms' tumor. In some embodiments, the cancer is a virus-associated cancer. In some embodiments, the cancer is a human papilloma virus (HPV)-associated cancer (e.g., an HPV-associated cancer of the back of the throat, cervix, anus, vulva, penis, and/or vagina). In some embodiments, the cancer is not skin cancer (e.g., melanoma, basal cell carcinoma, squamous cell carcinoma, actinic keratosis, an atypical mole, and/or Merkel cell carcinoma). In some embodiments, the cancer is not melanoma. In some embodiments, the subject suffers from one or more of carcinoma, lymphoma, blastoma, sarcoma, a neuroendocrine tumor, mesothelioma, schwannoma, meningioma, adenocarcinoma, melanoma, leukemia, and lymphoid malignancy. In some embodiments, the subject suffers from one or more of a solid tumor, a hematologic cancer, bladder cancer, brain cancer, breast cancer, colon cancer, gastric cancer, glioma, head cancer, leukemia, liver cancer, lung cancer, lymphoma, myeloma, neck cancer, ovarian cancer, melanoma, pancreatic cancer, renal cancer, salivary cancer, skin cancer, stomach cancer, thymic epithelial cancer, and thyroid cancer. In some embodiments, the subject suffers from one or more of small-cell lung cancer, non-small cell lung cancer, adenocarcinoma of the lung, or squamous carcinoma of the lung. In some embodiments, the subject suffers from osteosarcoma.

Other aspects of the present disclosure relate to a method of providing prophylactic, palliative, or therapeutic relief to one or more signs or symptoms of cancer in a subject in need thereof comprising administering to the subject an effective amount of any of the polynucleotides, recombinant nucleic acids comprising the polynucleotides, gene delivery vehicles comprising the polynucleotides and/or recombinant nucleic acids, medicaments, and/or pharmaceutical compositions or formulations described herein. In some embodiments, the subject is a human. In some embodiments, the subject suffers from a cancer. In some embodiments, the cancer is selected from acute myeloid leukemia (LAML or AML), acute lymphoblastic leukemia (ALL), adrenocortical carcinoma (ACC), bladder urothelial cancer (BLCA), brain stem glioma, brain lower grade glioma (LGG), brain tumor, breast cancer (BRCA), bronchial tumors, Burkitt lymphoma, cancer of unknown primary site, carcinoid tumor, carcinoma of unknown primary site, central nervous system atypical teratoid/rhabdoid tumor, central nervous system embryonal tumors, cervical squamous cell carcinoma, endocervical adenocarcinoma (CESC) cancer, childhood cancers, cholangiocarcinoma (CHOL), chordoma, chronic lymphocytic leukemia, chronic myelogenous leukemia, chronic myeloproliferative disorders, colon (adenocarcinoma) cancer (COAD), colorectal cancer, craniopharyngioma, cutaneous T-cell lymphoma, endocrine pancreas islet cell tumors, endometrial cancer, ependymoblastoma, ependymoma, esophageal cancer (ESCA), esthesioneuroblastoma, Ewing sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal cell tumor, gastrointestinal stromal tumor (GIST), gestational trophoblastic tumor, glioblastoma multiforme glioma GBM), hairy cell leukemia, head and neck cancer (HNSD), heart cancer, Hodgkin lymphoma, hypopharyngeal cancer, intraocular melanoma, islet cell tumors, Kaposi sarcoma, kidney cancer, Langerhans cell histiocytosis, laryngeal cancer, leptomeningeal cancer, lip cancer, liver cancer, Lymphoid Neoplasm Diffuse Large B-cell Lymphoma [DLBCL), malignant fibrous histiocytoma bone cancer, medulloblastoma, medulloepithelioma, melanoma, Merkel cell carcinoma, Merkel cell skin carcinoma, mesothelioma (MESO), metastatic squamous neck cancer with occult primary, mouth cancer, multiple endocrine neoplasia syndromes, multiple myeloma, multiple myeloma/plasma cell neoplasm, mycosis fungoides, myelodysplastic syndromes, myeloproliferative neoplasms, nasal cavity cancer, nasopharyngeal cancer, neuroblastoma, Non-Hodgkin lymphoma, nonmelanoma skin cancer, non-small cell lung cancer, oral cancer, oral cavity cancer, oropharyngeal cancer, osteosarcoma, other brain and spinal cord tumors, ovarian cancer, ovarian epithelial cancer, ovarian germ cell tumor, ovarian low malignant potential tumor, pancreatic cancer, papillomatosis, paranasal sinus cancer, parathyroid cancer, pelvic cancer, penile cancer, pharyngeal cancer, pheochromocytoma and paraganglioma (PCPG), pineal parenchymal tumors of intermediate differentiation, pineoblastoma, pituitary tumor, plasma cell neoplasm/multiple myeloma, pleuropulmonary blastoma, primary central nervous system (CNS) lymphoma, primary hepatocellular liver cancer, prostate cancer such as prostate adenocarcinoma (PRAD), rectal cancer, renal cancer, renal cell (kidney) cancer, renal cell cancer, respiratory tract cancer, retinoblastoma, rhabdomyosarcoma, salivary gland cancer, sarcoma (SARC), Sezary syndrome, skin cutaneous melanoma (SKCM), small cell lung cancer, small intestine cancer, soft tissue sarcoma, squamous cell carcinoma, squamous neck cancer, stomach (gastric) cancer, supratentorial primitive neuroectodermal tumors, T-cell lymphoma, testicular cancer testicular germ cell tumors (TGCT), throat cancer, thymic carcinoma, thymoma (THYM), thyroid cancer (THCA), transitional cell cancer, transitional cell cancer of the renal pelvis and ureter, trophoblastic tumor, ureter cancer, urethral cancer, uterine cancer, uterine cancer, uveal melanoma (UVM), vaginal cancer, vulvar cancer, Waldenstrom macroglobulinemia, and/or Wilms' tumor. In some embodiments, the cancer is a virus-associated cancer. In some embodiments, the cancer is a human papilloma virus (HPV)-associated cancer (e.g., an HPV-associated cancer of the back of the throat, cervix, anus, vulva, penis, and/or vagina). In some embodiments, the cancer is not skin cancer (e.g., melanoma, basal cell carcinoma, squamous cell carcinoma, actinic keratosis, an atypical mole, and/or Merkel cell carcinoma). In some embodiments, the cancer is not melanoma. In some embodiments, the subject suffers from one or more of carcinoma, lymphoma, blastoma, sarcoma, a neuroendocrine tumor, mesothelioma, schwannoma, meningioma, adenocarcinoma, melanoma, leukemia, and lymphoid malignancy. In some embodiments, the subject suffers from one or more of a solid tumor, a hematologic cancer, bladder cancer, brain cancer, breast cancer, colon cancer, gastric cancer, glioma, head cancer, leukemia, liver cancer, lung cancer, lymphoma, myeloma, neck cancer, ovarian cancer, melanoma, pancreatic cancer, renal cancer, salivary cancer, stomach cancer, thymic epithelial cancer, and thyroid cancer. In some embodiments, the subject suffers from one or more of small-cell lung cancer, non-small cell lung cancer, adenocarcinoma of the lung, or squamous carcinoma of the lung. In some embodiments, the subject suffers from osteosarcoma.

### VIII. HOST CELLS

Certain aspects of the present disclosure relate to one or more host cells comprising any of the polynucleotides and/or recombinant nucleic acids described herein. Any suitable host cell (prokaryotic or eukaryotic) known in the art may be used, including, for example: prokaryotic cells including eubacteria, such as Gram-negative or Gram-positive organisms, for example Enterobacteriaceae such as Escherichia (e.g., E. coli), Enterobacter, Erminia, Klebsiella, Proteus, Salmonella (e.g., S. typhimurium), Serratia (e.g., S. marcescans), and Shigella, as well as Bacilli such as B. subtilis and B. licheniformis; fungal cells (e.g., S. cerevisiae); insect cells (e.g., S2 cells, etc.); and mammalian cells, including monkey kidney CV1 line transformed by SV40 (COS-7, ATCC CRL 1651), human embryonic kidney line (293 or 293 cells subcloned for growth in suspension culture), baby hamster kidney cells (BHK, ATCC CCL 10), mouse Sertoli cells (TM4), monkey kidney cells (CV1 ATCC CCL 70), African green monkey kidney cells (VERO-76, ATCC CRL-1587), human cervical carcinoma cells (HELA, ATCC CCL 2), canine kidney cells (MDCK, ATCC CCL 34), buffalo rat liver cells (BRL 3A, ATCC CRL 1442), human lung cells (W138, ATCC CCL 75), human liver cells (Hep G2, HB 8065), mouse mammary tumor (MMT 060562, ATCC CCL51), TRI cells, MRC 5 cells, FS4 cells, human hepatoma line (Hep G2), Chinese hamster ovary (CHO) cells, including DHFR″ CHO cells, and myeloma cell lines such as NSO and Sp2/0. In some embodiments, the host cell is a human cell (e.g., a T cell, NK cell, etc.) or non-human primate cell. In some embodiments, the host cells are cells from a cell line. Examples of suitable host cells or cell lines may include, but are not limited to, 293, HeLa, SH-Sy5y, Hep G2, CACO-2, A549, L929, 3T3, K562, CHO-K1, MDCK, HUVEC, Vero, N20, COS-7, PSN1, VcaP, CHO cells, and the like.

### IX. ARTICLES OF MANUFACTURE OR KITS

Certain aspects of the present disclosure relate to an article of manufacture or a kit comprising any of the polynucleotides, recombinant nucleic acids comprising the polynucleotides, gene delivery vehicles comprising the polynucleotides and/or recombinant nucleic acids, medicaments, and/or pharmaceutical compositions or formulations described herein. In some embodiments, the article of manufacture or kit comprises a package insert comprising instructions for administering the polynucleotides, recombinant nucleic acids comprising the polynucleotides, gene delivery vehicles comprising the polynucleotides and/or recombinant nucleic acids, medicaments, and/or pharmaceutical compositions or formulations.

Suitable containers for the polynucleotides, recombinant nucleic acids comprising the polynucleotides, gene delivery vehicles comprising the polynucleotides and/or recombinant nucleic acids, medicaments, and/or pharmaceutical compositions or formulations may include, for example, bottles, vials, bags, tubes, and syringes. The container may be formed from a variety of materials such as glass, plastic (such as polyvinyl chloride or polyolefin), or metal alloy (such as stainless steel or hastelloy). In some embodiments, the container comprises a label on, or associated with the container, wherein the label indicates directions for use. The article of manufacture or kit may further include other materials desirable from a commercial and user standpoint, including other buffers, diluents, filters, a package insert, and the like.

### X. ENUMERATED EMBODIMENTS

Embodiment 1: a composition comprising a first recombinant polynucleotide encoding an Interleukin (IL)-2 polypeptide and a second recombinant polynucleotide encoding an IL-12 polypeptide.

Embodiment 2: a pharmaceutical composition comprising the first recombinant polynucleotide and the second recombinant polynucleotide of embodiment 1.

Embodiment 3: the pharmaceutical composition of embodiment 2, wherein the pharmaceutical composition comprises a recombinant nucleic acid comprising the first polynucleotide and a recombinant nucleic acid comprising the second polynucleotide.

Embodiment 4: the pharmaceutical composition of any of embodiments 2 or 3, wherein the recombinant nucleic acid comprising the first polynucleotide and the recombinant nucleic acid comprising the second polynucleotide are the same recombinant nucleic acid.

Embodiment 5: the pharmaceutical composition of any of embodiments 2-4, wherein the recombinant nucleic acid comprising the first polynucleotide and the recombinant nucleic acid comprising the second polynucleotide are different recombinant nucleic acids.

Embodiment 6: the pharmaceutical composition of any of embodiments 2-5, wherein the pharmaceutical composition comprises one or more gene delivery vehicles comprising the first polynucleotide and the second polynucleotide.

Embodiment 7: the pharmaceutical composition of any of embodiments 2-6, wherein the first polynucleotide and the second polynucleotide are contiguous.

Embodiment 8: the pharmaceutical composition of any of embodiments 2-7, wherein the first polynucleotide and the second polynucleotide are non-contiguous.

Embodiment 9: the pharmaceutical composition of any of embodiments 2-8, wherein the first polynucleotide and/or the second polynucleotide comprises a deoxyribonucleic acid (DNA).

Embodiment 10: the pharmaceutical composition of any of embodiments 2-9, wherein the first polynucleotide or the second polynucleotide comprises a deoxyribonucleic acid (DNA).

Embodiment 11: the pharmaceutical composition of any of embodiments 2-10, wherein the first polynucleotide and the second polynucleotide comprises a deoxyribonucleic acid (DNA).

Embodiment 12: the pharmaceutical composition of any of embodiments 2-11, wherein the first polynucleotide comprises a deoxyribonucleic acid (DNA).

Embodiment 13: the pharmaceutical composition of any of embodiments 2-12, wherein the second polynucleotide comprises a deoxyribonucleic acid (DNA).

Embodiment 14: the pharmaceutical composition of any of embodiments 2-13, wherein the DNA is a linear DNA or a circular DNA.

Embodiment 15: the pharmaceutical composition of any of embodiments 2-14, wherein the DNA is a linear DNA.

Embodiment 16: the pharmaceutical composition of any of embodiments 2-15, wherein the DNA is a circular DNA.

Embodiment 17: the pharmaceutical composition of any of embodiments 2-16, wherein the first polynucleotide and/or the second polynucleotide comprises a ribonucleic acid (RNA), a messenger RNA (mRNA), or a modified mRNA (mmRNA).

Embodiment 18: the pharmaceutical composition of any of embodiments 2-17, wherein the first polynucleotide comprises an RNA.

Embodiment 19: the pharmaceutical composition of any of embodiments 2-18, wherein the first polynucleotide comprises an mRNA.

Embodiment 20: the pharmaceutical composition of any of embodiments 2-19, wherein the first polynucleotide comprises an mmRNA.

Embodiment 21: the pharmaceutical composition of any of embodiments 2-20, wherein the second polynucleotide comprises an RNA.

Embodiment 22: the pharmaceutical composition of any of embodiments 2-21, wherein the second polynucleotide comprises an mRNA.

Embodiment 23: the pharmaceutical composition of any of embodiments 2-22, wherein the second polynucleotide comprises an mmRNA.

Embodiment 24: the pharmaceutical composition of any of embodiments 2-23, wherein the first polynucleotide and the second polynucleotide comprise an RNA.

Embodiment 25: the pharmaceutical composition of any of embodiments 2-24, wherein the first polynucleotide and the second polynucleotide comprise an mRNA.

Embodiment 26: the pharmaceutical composition of any of embodiments 2-25, wherein the first polynucleotide and the second polynucleotide comprise an mmRNA.

Embodiment 27: the pharmaceutical composition of any of embodiments 2-26, wherein the mRNA and/or the mmRNA further comprises a 5′ untranslated region (UTR), a 3′ UTR, a polyadenylation (poly(A)) tail, and/or a 5′ cap analog.

Embodiment 28: the pharmaceutical composition of any of embodiments 2-27, wherein the mRNA further comprises a 5′ untranslated region (UTR), a 3′ UTR, a polyadenylation (poly(A)) tail, and a 5′ cap analog.

Embodiment 29: the pharmaceutical composition of any of embodiments 2-28, wherein the mmRNA further comprises a 5′ untranslated region (UTR), a 3′ UTR, a polyadenylation (poly(A)) tail, and a 5′ cap analog.

Embodiment 30: the pharmaceutical composition of any of embodiments 2-29, wherein the 5′ cap analog is 7 mG (5′) ppp (5′) NImpNp.

Embodiment 31: the pharmaceutical composition of any of embodiments 2-30, wherein the mRNA and/or the mmRNA further comprises a 1-methylpseudouridine modification and/or a 1-ethylpseudouridine modification.

Embodiment 32: the pharmaceutical composition of any of embodiments 2-31, wherein the mRNA further comprises a 1-methylpseudouridine modification.

Embodiment 33: the pharmaceutical composition of any of embodiments 2-32, wherein the mRNA further comprises a 1-ethylpseudouridine modification.

Embodiment 34: the pharmaceutical composition of any of embodiments 1-33, wherein the mmRNA further comprises a 1-methylpseudouridine modification.

Embodiment 35: the pharmaceutical composition of any of embodiments 2-34, wherein the mmRNA further comprises a 1-ethylpseudouridine modification.

Embodiment 36: the pharmaceutical composition of any of embodiments 2-35, wherein the mRNA further comprises a 1-methylpseudouridine modification and a 1-ethylpseudouridine modification.

Embodiment 37: the pharmaceutical composition of any of embodiments 2-36, wherein the mmRNA further comprises a 1-methylpseudouridine modification and a 1-ethylpseudouridine modification.

Embodiment 38: the pharmaceutical composition of any of embodiments 2-37, wherein

the first polynucleotide and/or the second polynucleotide are codon-optimized.

Embodiment 39: the pharmaceutical composition of any of embodiments 2-38, wherein the first polynucleotide is codon-optimized.

Embodiment 40: the pharmaceutical composition of any of embodiments 2-29, wherein the second polynucleotide is codon-optimized.

Embodiment 41: the pharmaceutical composition of any of embodiments 2-40, wherein the first polynucleotide and the second polynucleotide are codon-optimized.

Embodiment 42: the pharmaceutical composition of any of embodiments 2-41, wherein the first polynucleotide and/or the second polynucleotide are codon-optimized for human codon usage.

Embodiment 43: the pharmaceutical composition of any of embodiments 2-42, wherein the first polynucleotide is codon-optimized for human codon usage.

Embodiment 44: the pharmaceutical composition of any of embodiments 2-43, wherein the second polynucleotide is codon-optimized for human codon usage.

Embodiment 45: the pharmaceutical composition of any of embodiments 2-44, wherein the first polynucleotide and the second polynucleotide are codon-optimized for human codon usage.

Embodiment 46: the pharmaceutical composition of any of embodiments 2-45, wherein the IL-2 polypeptide is a human IL-2 polypeptide.

Embodiment 47: the pharmaceutical composition of any of embodiments 2-46, wherein the IL-2 polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 1.

Embodiment 48: the pharmaceutical composition of any of embodiments 2-47, wherein the IL-12 polypeptide is a human IL-12 polypeptide.

Embodiment 49: the pharmaceutical composition of any of embodiments 2-48, wherein the IL-12 polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID Nos: 2-4.

Embodiment 50: the pharmaceutical composition of any of embodiments 2-49, wherein the IL-12 polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 2.

Embodiment 51: the pharmaceutical composition of any of embodiments 2-50, wherein the IL-12 polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 3.

Embodiment 52: the pharmaceutical composition of any of embodiments 2-51, wherein the IL-12 polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 4.

Embodiment 53: the pharmaceutical composition of any of embodiments 2-52, wherein the one or more gene delivery vehicles are one or more of a viral gene delivery vehicle and/or a non-viral gene delivery vehicle.

Embodiment 54: the pharmaceutical composition of any of embodiments 2-53, wherein the one or more gene delivery vehicles is one or more viral gene delivery vehicle.

Embodiment 55: the pharmaceutical composition of any of embodiments 2-54, wherein the one or more gene delivery vehicles is one or more of a non-viral gene delivery vehicle.

Embodiment 56: the pharmaceutical composition of any of embodiments 2-55, wherein the one or more gene delivery vehicles are one or more of a viral gene delivery vehicle and a non-viral gene delivery vehicle.

Embodiment 57: the pharmaceutical composition of any of embodiments 2-56, wherein the viral gene delivery vehicle comprises a recombinant viral genome.

Embodiment 58: the pharmaceutical composition of any of embodiments 2-57, wherein the recombinant viral genome comprises the first polynucleotide and/or the second polynucleotide.

Embodiment 59: the pharmaceutical composition of any of embodiments 2-58, wherein the recombinant viral genome comprises the first polynucleotide.

Embodiment 60: the pharmaceutical composition of any of embodiments 2-59, wherein the recombinant viral genome comprises the second polynucleotide.

Embodiment 61: the pharmaceutical composition of any of embodiments 2-60, wherein the recombinant viral genome comprises the first polynucleotide and the second polynucleotide.

Embodiment 62: the pharmaceutical composition of any of embodiments 2-61, wherein the recombinant viral genome is replication competent or replication defective.

Embodiment 63: the pharmaceutical composition of any of embodiments 2-62, wherein the recombinant viral genome is replication competent.

Embodiment 64: the pharmaceutical composition of any of embodiments 2-63, wherein the recombinant viral genome is replication defective.

Embodiment 65: the pharmaceutical composition of any of embodiments 2-64, wherein the recombinant viral genome is a recombinant oncolytic virus genome, or wherein the recombinant viral genome is not a recombinant oncolytic virus genome.

Embodiment 66: the pharmaceutical composition of any of embodiments 2-65, wherein the recombinant viral genome is a recombinant oncolytic virus genome.

Embodiment 67: the pharmaceutical composition of any of embodiments 2-66, wherein the recombinant viral genome is not a recombinant oncolytic virus genome.

Embodiment 68: the pharmaceutical composition of any of embodiments 2-67, wherein the recombinant viral genome is selected from the group consisting of a recombinant adenovirus genome, a recombinant retrovirus genome, a recombinant adeno-associated virus genome, a recombinant herpes virus genome, a recombinant poxvirus genome, a recombinant bacteriophage genome, a recombinant alphavirus genome, a recombinant picornavirus genome, a recombinant iridovirus genome, a recombinant Newcastle disease virus genome, a recombinant baculovirus genome, a recombinant geminivirus genome, a recombinant caulimovirus genome, and a combination thereof.

Embodiment 69: the pharmaceutical composition of any of embodiments 2-68, wherein the recombinant viral genome is a recombinant adenovirus genome.

Embodiment 70: the pharmaceutical composition of any of embodiments 2-69, wherein the recombinant viral genome is a recombinant retrovirus genome.

Embodiment 71: the pharmaceutical composition of any of embodiments 2-70, wherein the recombinant viral genome is a recombinant adeno-associated virus genome.

Embodiment 72: the pharmaceutical composition of any of embodiments 2-71, wherein the recombinant viral genome is a recombinant herpes virus genome.

Embodiment 73: the pharmaceutical composition of any of embodiments 2-72, wherein the recombinant viral genome is a recombinant poxvirus genome.

Embodiment 74: the pharmaceutical composition of any of embodiments 2-73, wherein the recombinant viral genome is a recombinant bacteriophage genome.

Embodiment 75: the pharmaceutical composition of any of embodiments 2-74, wherein the recombinant viral genome is a recombinant alphavirus genome.

Embodiment 76: the pharmaceutical composition of any of embodiments 2-75, wherein the recombinant viral genome is a recombinant picornavirus genome.

Embodiment 77: the pharmaceutical composition of any of embodiments 2-76, wherein the recombinant viral genome is a recombinant iridovirus genome.

Embodiment 78: the pharmaceutical composition of any of embodiments 2-77, wherein the recombinant viral genome is a recombinant Newcastle disease virus genome.

Embodiment 79: the pharmaceutical composition of any of embodiments 2-78, wherein the recombinant viral genome is a recombinant baculovirus genome.

Embodiment 80: the pharmaceutical composition of any of embodiments 2-79, wherein the recombinant viral genome is a recombinant geminivirus genome.

Embodiment 81: the pharmaceutical composition of any of embodiments 2-80, wherein the recombinant viral genome is a recombinant caulimovirus genome.

Embodiment 82: the pharmaceutical composition of any of embodiments 2-81, wherein the recombinant adenovirus genome is selected from the group consisting of a recombinant Atadenovirus genome, a recombinant Aviadenovirus genome, a recombinant Ichtadenovirus genome, a recombinant Mastadenovirus genome, a recombinant Siadenovirus genome, a recombinant Testadenovirus genome, and any derivatives thereof.

Embodiment 83: the pharmaceutical composition of any of embodiments 2-82, wherein the recombinant retrovirus genome is selected from the group consisting of a recombinant Alpharetrovirus genome, a recombinant Betaretrovirus genome, a recombinant Gammaretrovirus genome, a recombinant Deltaretrovirus genome, a recombinant Epsilonretrovirus genome, a recombinant Lentivirus genome, a recombinant Bovispumavirus genome, a recombinant Equispumavirus genome, a recombinant Felispumavirus genome, a recombinant Prosimiispumavirus genome, a recombinant Simiispumavirus genome, and any derivatives thereof.

Embodiment 84: the pharmaceutical composition of any of embodiments 2-83, wherein the recombinant adeno-associated virus genome is selected from the group consisting of a recombinant Adeno-associated dependoparvovirus A genome, a recombinant Adeno-associated dependoparvovirus B genome, and any derivates thereof.

Embodiment 85: the pharmaceutical composition of any of embodiments 2-84, wherein the recombinant adeno-associated virus (AAV) genome is a recombinant AAV serotype 1 genome, a recombinant AAV serotype 2 genome, a recombinant AAV serotype 3 genome, a recombinant AAV serotype 4 genome, a recombinant AAV serotype 5 genome, a recombinant AAV serotype 6 genome, a recombinant AAV serotype 7 genome, a recombinant AAV serotype 8 genome, a recombinant AAV serotype 9 genome, a recombinant AAV serotype 10 genome. a recombinant AAV serotype 11 genome, or a combination thereof.

Embodiment 86: the pharmaceutical composition of any of embodiments 2-85, wherein the recombinant herpes virus genome is selected from the group consisting of a recombinant herpes simplex virus genome, a recombinant varicella zoster virus genome, a recombinant human cytomegalovirus genome, a recombinant herpesvirus 6A genome, a recombinant herpesvirus 6B genome, a recombinant herpesvirus 7 genome, a recombinant Epstein-Barr virus genome, a recombinant Kaposi's sarcoma-associated herpesvirus genome, and any derivatives thereof.

Embodiment 87: the pharmaceutical composition of any of embodiments 2-86, wherein the recombinant herpes simplex virus genome is a recombinant herpes simplex virus type 1 (HSV-1) genome, a recombinant herpes simplex virus type 2 (HSV-2) genome, or any derivatives thereof.

Embodiment 88: the pharmaceutical composition of any of embodiments 2-87, wherein the recombinant poxvirus genome is selected from the group consisting of a recombinant smallpox virus genome, a recombinant vaccinia virus genome, a recombinant cowpox virus genome, a recombinant monkeypox virus genome, a recombinant orf virus genome, a recombinant pseudocowpox virus genome, a recombinant bovine papular stomatitis virus genome, a recombinant tanapox virus genome, a recombinant yaba monkey tumor virus genome, a recombinant molluscum contagiosum virus genome, and any derivatives thereof.

Embodiment 89: the pharmaceutical composition of any of embodiments 2-88, wherein the recombinant bacteriophage genome is selected from the group consisting of a recombinant 186 phage genome, a recombinant Escherichia virus Lambda genome, a recombinant Pseudomonas virus phi6 genome, a recombinant Bacillus virus φ29 genome, a recombinant Escherichia virus φX174 genome, a recombinant Bacteriophage φCb5 genome, a recombinant Escherichia virus G4 genome, a recombinant Escherichia virus M13 genome, a recombinant Emesvirus zinderi genome, a recombinant N4 phage genome, a recombinant Escherichia virus P1 genome, a recombinant Escherichia virus P2 genome, a recombinant Enterobacteria phage P4 genome, a recombinant R17 phage genome, a recombinant Enterobacteria phage T2 genome, a recombinant Escherichia virus T4 genome, a recombinant Escherichia virus T7 genome, a recombinant Bacteriophage T12 genome, and any derivatives thereof.

Embodiment 90: the pharmaceutical composition of any of embodiments 2-89, wherein the recombinant alphavirus genome is selected from the group consisting of a recombinant Aura virus genome, a recombinant Barmah Forest virus genome, a recombinant Bebaru virus genome, a recombinant Caaingua virus genome, a recombinant Cabassou virus genome, a recombinant Chikungunya virus genome, a recombinant Eastern equine encephalitis virus genome, a recombinant Eilat virus genome, a recombinant Everglades virus genome, a recombinant Fort Morgan virus genome, a recombinant Getah virus genome, a recombinant Highlands J virus genome, a recombinant Madariaga virus genome, a recombinant Mayaro virus genome, a recombinant Middelburg virus genome, a recombinant Mosso das Pedras virus genome, a recombinant Mucambo virus genome, a recombinant Ndumu virus genome, a recombinant O'nyong'nyong virus genome, a recombinant Pixuna virus genome, a recombinant Rio Negro virus genome, a recombinant Ross River virus genome, a recombinant Salmon pancreas disease virus genome, a recombinant Semliki Forest virus genome, a recombinant Sindbis virus genome, a recombinant Southern elephant seal virus genome, a recombinant Tonate virus genome, a recombinant Trocara virus genome, a recombinant Una virus genome, a recombinant Venezuelan equine encephalitis virus genome, a recombinant Western equine encephalitis virus genome, a recombinant Whataroa virus genome, and any derivatives thereof.

Embodiment 91: the pharmaceutical composition of any of embodiments 2-90, wherein the recombinant picornavirus genome is selected from the group consisting of a recombinant Aalivirus genome, a recombinant Ailurivirus genome, a recombinant Ampivirus genome, a recombinant Anativirus genome, a recombinant Aphthovirus genome, a recombinant Aquamavirus genome, a recombinant Avihepatovirus genome, a recombinant Avisivirus genome, a recombinant Boosepivirus genome, a recombinant Bopivirus genome, a recombinant Caecilivirus genome, a recombinant Cardiovirus genome, a recombinant Cosavirus genome, a recombinant Crahelivirus genome, a recombinant Crohivirus genome, a recombinant Danipivirus genome, a recombinant Dicipivirus genome, a recombinant Diresapivirus genome, a recombinant Enterovirus genome, a recombinant Erbovirus genome, a recombinant Felipivirus genome, a recombinant Fipivirus genome, a recombinant Gallivirus genome, a recombinant Gruhelivirus genome, a recombinant Grusopivirus genome, a recombinant Harkavirus genome, a recombinant Hemipivirus genome, a recombinant Hepatovirus genome, a recombinant Hunnivirus genome, a recombinant Kobuvirus genome, a recombinant Kunsagivirus genome, a recombinant Limnipivirus genome, a recombinant Livupivirus genome, a recombinant Ludopivirus genome, a recombinant Malagasivirus genome, a recombinant Marsupivirus genome, a recombinant Megrivirus genome, a recombinant Mischivirus genome, a recombinant Mosavirus genome, a recombinant Mupivirus genome, a recombinant Myrropivirus genome, a recombinant Orivirus genome, a recombinant Oscivirus genome, a recombinant Parabovirus genome, a recombinant Parechovirus genome, a recombinant Pasivirus genome, a recombinant Passerivirus genome, a recombinant Pemapivirus genome, a recombinant Poecivirus genome, a recombinant Potamipivirus genome, a recombinant Pygoscepivirus genome, a recombinant Rabovirus genome, a recombinant Rafivirus genome, a recombinant Rajidapivirus genome, a recombinant Rohelivirus genome, a recombinant Rosavirus genome, a recombinant Sakobuvirus genome, a recombinant Salivirus genome, a recombinant Sapelovirus genome, a recombinant Senecavirus genome, a recombinant Shanbavirus genome, a recombinant Sicinivirus genome, a recombinant Symapivirus genome, a recombinant Teschovirus genome, a recombinant Torchivirus genome, a recombinant Tottorivirus genome, a recombinant Tremovirus genome, a recombinant Tropivirus genome, and any derivatives thereof.

Embodiment 92: the pharmaceutical composition of any of embodiments 2-91, wherein the recombinant iridovirus genome is a recombinant Invertebrate iridescent virus 6 (IIV-6) genome or a recombinant Invertebrate iridescent virus 31 (IIV-31) genome.

Embodiment 93: the pharmaceutical composition of any of embodiments 2-92, wherein the recombinant baculovirus genome is selected from the group consisting of a recombinant alphabaculovirus genome, a recombinant betabaculovirus genome, a recombinant deltabaculovirus genome, a recombinant gammabaculovirus genome, and any derivatives thereof.

Embodiment 94: the pharmaceutical composition of any of embodiments 2-93, wherein the recombinant geminivirus genome is selected from the group consisting of a recombinant Becurtovirus genome, a recombinant Begomovirus genome, a recombinant Capulavirus genome, a recombinant Citlodavirus genome, a recombinant Curtovirus genome, a recombinant Eragrovirus genome, a recombinant Grablovirus genome, a recombinant Maldovirus genome, a recombinant Mastrevirus genome, a recombinant Mulcrilevirus genome, a recombinant Opunvirus genome, a recombinant Topilevirus genome, a recombinant Topocuvirus genome, a recombinant Turncurtovirus genome, and any derivatives thereof.

Embodiment 95: the pharmaceutical composition of any of embodiments 2-94, wherein the recombinant caulimovirus genome is selected from the group consisting of a recombinant Angelica bushy stunt virus genome, a recombinant Atractylodes mild mottle virus genome, a recombinant Carnation etched ring virus genome, a recombinant Cauliflower mosaic virus genome, a recombinant Dahlia mosaic virus genome, a recombinant Figwort mosaic virus genome, a recombinant Horseradish latent virus genome, a recombinant Lamium leaf distortion virus genome, a recombinant Mirabilis mosaic virus genome, a recombinant Soybean Putnam virus genome, a recombinant Strawberry vein banding virus genome, a recombinant Thistle mottle virus genome, and any derivatives thereof.

Embodiment 96: the pharmaceutical composition of any of embodiments 2-95, wherein the gene delivery vehicle is the non-viral gene delivery vehicle.

Embodiment 97: the pharmaceutical composition of any of embodiments 2-96, wherein the non-viral gene delivery vehicle is a chemical compound, a bacterium, a mammalian cell, or a physical delivery system.

Embodiment 98: the pharmaceutical composition of any of embodiments 2-97, wherein the non-viral gene delivery vehicle is a chemical compound.

Embodiment 99: the pharmaceutical composition of any of embodiments 2-98, wherein the non-viral gene delivery vehicle is a bacterium.

Embodiment 100: the pharmaceutical composition of any of embodiments 2-99, wherein the non-viral gene delivery vehicle is a mammalian cell.

Embodiment 101: the pharmaceutical composition of any of embodiments 2-100, wherein the non-viral gene delivery vehicle is a physical delivery system.

Embodiment 102: the pharmaceutical composition of any of embodiments 2-101, wherein the chemical compound is selected from the groups consisting of a polymer compound, a lipid compound, an inorganic compound, and a combination thereof.

Embodiment 103: the pharmaceutical composition of any of embodiments 2-102, wherein the chemical compound is a polymer compound.

Embodiment 104: the pharmaceutical composition of any of embodiments 2-103, wherein the chemical compound is a lipid compound.

Embodiment 105: the pharmaceutical composition of any of embodiments 2-104, wherein the chemical compound is an inorganic compound.

Embodiment 106: the pharmaceutical composition of any of embodiments 2-105, wherein the polymer compound is a natural polymer, a synthetic polymer, a biopolymer, a biodegradable polymer, a cationic polymer, a protein polymer, a polysaccharide polymer, or any combination thereof.

Embodiment 107: the pharmaceutical composition of any of embodiments 2-106, wherein the polymer compound is selected from the group consisting of poly-L-lactide (PLA), chitosan, pectin, polyethylenimine (PEI), poly-L-lysine (PLL), poly 2-N-dimethylaminoethyl methacrylate (PDMAEMA), polyaminoamine, polyaminoesters, polyamidoamine, poly-vinylimidazole (PVI), Poly-beta-amino ester (PBAE), gelatin, albumin, chitosan, beta-cyclodextrin, dextran, hyaluronic acid, polyester, alpha-Pyrrolidinohexiophenone (PHP), Poly-2-acrylamidoglycolic acid (PAGA), Poly-delta-valerolactone (PVL), polyphenyl ethers (PPE), Poly-beta-amino-ester (PBAE), poly-amine-co-ester (PACE), poly-lactic-co-glycolic acid (PLGA), polycarbonates, polyurethanes, micelles, cationic emulsion, exosome, Reb Blood Cell membrane, DNA-protein complexes, plasmid DNA, naked DNA, pBR322, pUC, and any derivatives thereof.

Embodiment 108: the pharmaceutical composition of any of embodiments 2-107, wherein the lipid compound is selected from the group consisting of a nanoparticle, a lipid nanoparticle, a liposome, a cationic liposome, a solid lipid nanoparticle, a lipid emulsion, a lipidoid, a cytofectin, a lipid emulsion, a surfactant, a gemini surfactant, and any derivatives thereof.

Embodiment 109: the pharmaceutical composition of any of embodiments 2-108, wherein the inorganic compound is selected from the group consisting of a nanocarrier, a DNA nanclew, a gold nanoparticle, a carbon nanotube, a graphene, a quantum dot, an up-conversion nanoparticle, a silica nanoparticle, an iron oxide, a ferritin, and any derivatives thereof.

Embodiment 110: the pharmaceutical composition of any of embodiments 2-109, wherein the physical delivery system is selected from the group consisting of electroporation, gene gun, jet gun, ultrasound, nucleofection, hydrodynamic gene delivery, needle injection, microinjection, ballistic DNA injection, sonoporation, photoporation, laser pulse, magnetofection, magnetoporation, magnetic particles, hydroporation, and any combination thereof.

Embodiment 111: use of the pharmaceutical composition of any of embodiments 2-110 in the manufacture of a medicament for treating cancer.

Embodiment 112: use of embodiment 111, wherein the cancer is selected from the group consisting of acute myeloid leukemia, acute lymphoblastic leukemia, adrenocortical carcinoma, bladder urothelial cancer, brain stem glioma, brain lower grade glioma, brain tumor, breast cancer, bronchial tumors, Burkitt lymphoma, cancer of unknown primary site, carcinoid tumor, carcinoma of unknown primary site, central nervous system atypical teratoid/rhabdoid tumor, central nervous system embryonal tumors, cervical squamous cell carcinoma, endocervical adenocarcinoma cancer, childhood cancers, cholangiocarcinoma, chordoma, chronic lymphocytic leukemia, chronic myelogenous leukemia, chronic myeloproliferative disorders, colon cancer, colorectal cancer, craniopharyngioma, cutaneous T-cell lymphoma, endocrine pancreas islet cell tumors, endometrial cancer, ependymoblastoma, ependymoma, esophageal cancer, esthesioneuroblastoma, Ewing sarcoma, extracranial germ cell tumor, extragonadal germ cell tumor, extrahepatic bile duct cancer, gallbladder cancer, gastric cancer, gastrointestinal carcinoid tumor, gastrointestinal stromal cell tumor, gastrointestinal stromal tumor, gestational trophoblastic tumor, glioblastoma multiforme glioma, hairy cell leukemia, head and neck cancer, heart cancer, Hodgkin lymphoma, hypopharyngeal cancer, intraocular melanoma, islet cell tumors, Kaposi sarcoma, kidney cancer, Langerhans cell histiocytosis, laryngeal cancer, leptomeningeal cancer, lip cancer, liver cancer, Lymphoid Neoplasm Diffuse Large B-cell Lymphoma, malignant fibrous histiocytoma bone cancer, medulloblastoma, medulloepithelioma, melanoma, Merkel cell carcinoma, Merkel cell skin carcinoma, mesothelioma, metastatic squamous neck cancer with occult primary, mouth cancer, multiple endocrine neoplasia syndromes, multiple myeloma, multiple myeloma/plasma cell neoplasm, mycosis fungoides, myelodysplastic syndromes, myeloproliferative neoplasms, nasal cavity cancer, nasopharyngeal cancer, neuroblastoma, Non-Hodgkin lymphoma, nonmelanoma skin cancer, non-small cell lung cancer, oral cancer, oral cavity cancer, oropharyngeal cancer, osteosarcoma, other brain and spinal cord tumors, ovarian cancer, ovarian epithelial cancer, ovarian germ cell tumor, ovarian low malignant potential tumor, pancreatic cancer, papillomatosis, paranasal sinus cancer, parathyroid cancer, pelvic cancer, penile cancer, pharyngeal cancer, pheochromocytoma and paraganglioma, pineal parenchymal tumors of intermediate differentiation, pineoblastoma, pituitary tumor, plasma cell neoplasm/multiple myeloma, pleuropulmonary blastoma, primary central nervous system lymphoma, primary hepatocellular liver cancer, prostate cancer such as prostate adenocarcinoma, rectal cancer, renal cancer, renal cell cancer, renal cell cancer, respiratory tract cancer, retinoblastoma, rhabdomyosarcoma, salivary gland cancer, sarcoma, Sezary syndrome, skin cutaneous melanoma, small cell lung cancer, small intestine cancer, soft tissue sarcoma, squamous cell carcinoma, squamous neck cancer, stomach cancer, supratentorial primitive neuroectodermal tumors, T-cell lymphoma, testicular cancer testicular germ cell tumors, throat cancer, thymic carcinoma, thymoma, thyroid cancer, transitional cell cancer, transitional cell cancer of the renal pelvis and ureter, trophoblastic tumor, ureter cancer, urethral cancer, uterine cancer, uterine cancer, uveal melanoma, vaginal cancer, vulvar cancer, Waldenstrom macroglobulinemia, and Wilms' tumor.

Embodiment 113: a method of expressing, enhancing, increasing, augmenting, and/or supplementing the levels of an IL-2 polypeptide and/or an IL-12 polypeptide in one or more cells of a subject, the method comprising administering to the subject an effective amount of the pharmaceutical composition of any of embodiment 2-112.

Embodiment 114: the method of embodiment 113, wherein the one or more cells are one or more cells of the respiratory tract, airway epithelial, and/or lung.

Embodiment 115: the method of any of embodiments 113 or 114, wherein the one or more cells is one or more cells of the respiratory tract.

Embodiment 116: the method of any of embodiments 113-115, wherein the one or more cells is one or more of the airway epithelial.

Embodiment 117: the method of any of embodiments 113-116, wherein the one or more cells is one or more of the lung.

Embodiment 118: a method of providing prophylactic, palliative, or therapeutic relief of one or more signs or symptoms of cancer in a subject in need thereof, the method comprising administering to the subject an effective amount of the pharmaceutical composition of any of embodiments 2-112.

Embodiment 119: a method of treating cancer in a subject in need thereof, the method comprising administering to the subject an effective amount of the pharmaceutical composition of any of embodiment 2-112.

Embodiment 120: the method of any of embodiments 118 or 119, wherein the cancer is selected from the group consisting of acute myeloid leukemia, acute lymphoblastic leukemia, adrenocortical carcinoma, bladder urothelial cancer, brain stem glioma, brain lower grade glioma, brain tumor, breast cancer, bronchial tumors, Burkitt lymphoma, cancer of unknown primary site, carcinoid tumor, carcinoma of unknown primary site, central nervous system atypical teratoid/rhabdoid tumor, central nervous system embryonal tumors, cervical squamous cell carcinoma, endocervical adenocarcinoma cancer, childhood cancers, cholangiocarcinoma, chordoma, chronic lymphocytic leukemia, chronic myelogenous leukemia, chronic myeloproliferative disorders, colon cancer, colorectal cancer, craniopharyngioma, cutaneous T-cell lymphoma, endocrine pancreas islet cell tumors, endometrial cancer, ependymoblastoma, ependymoma, esophageal cancer, esthesioneuroblastoma, Ewing sarcoma, extracranial germ cell tumor, extragonadal germ cell tumor, extrahepatic bile duct cancer, gallbladder cancer, gastric cancer, gastrointestinal carcinoid tumor, gastrointestinal stromal cell tumor, gastrointestinal stromal tumor, gestational trophoblastic tumor, glioblastoma multiforme glioma, hairy cell leukemia, head and neck cancer, heart cancer, Hodgkin lymphoma, hypopharyngeal cancer, intraocular melanoma, islet cell tumors, Kaposi sarcoma, kidney cancer, Langerhans cell histiocytosis, laryngeal cancer, leptomeningeal cancer, lip cancer, liver cancer, Lymphoid Neoplasm Diffuse Large B-cell Lymphoma, malignant fibrous histiocytoma bone cancer, medulloblastoma, medulloepithelioma, melanoma, Merkel cell carcinoma, Merkel cell skin carcinoma, mesothelioma, metastatic squamous neck cancer with occult primary, mouth cancer, multiple endocrine neoplasia syndromes, multiple myeloma, multiple myeloma/plasma cell neoplasm, mycosis fungoides, myelodysplastic syndromes, myeloproliferative neoplasms, nasal cavity cancer, nasopharyngeal cancer, neuroblastoma, Non-Hodgkin lymphoma, nonmelanoma skin cancer, non-small cell lung cancer, oral cancer, oral cavity cancer, oropharyngeal cancer, osteosarcoma, other brain and spinal cord tumors, ovarian cancer, ovarian epithelial cancer, ovarian germ cell tumor, ovarian low malignant potential tumor, pancreatic cancer, papillomatosis, paranasal sinus cancer, parathyroid cancer, pelvic cancer, penile cancer, pharyngeal cancer, pheochromocytoma and paraganglioma, pineal parenchymal tumors of intermediate differentiation, pineoblastoma, pituitary tumor, plasma cell neoplasm/multiple myeloma, pleuropulmonary blastoma, primary central nervous system lymphoma, primary hepatocellular liver cancer, prostate cancer such as prostate adenocarcinoma, rectal cancer, renal cancer, renal cell cancer, renal cell cancer, respiratory tract cancer, retinoblastoma, rhabdomyosarcoma, salivary gland cancer, sarcoma, Sezary syndrome, skin cutaneous melanoma, small cell lung cancer, small intestine cancer, soft tissue sarcoma, squamous cell carcinoma, squamous neck cancer, stomach cancer, supratentorial primitive neuroectodermal tumors, T-cell lymphoma, testicular cancer testicular germ cell tumors, throat cancer, thymic carcinoma, thymoma, thyroid cancer, transitional cell cancer, transitional cell cancer of the renal pelvis and ureter, trophoblastic tumor, ureter cancer, urethral cancer, uterine cancer, uterine cancer, uveal melanoma, vaginal cancer, vulvar cancer, Waldenstrom macroglobulinemia, and Wilms' tumor.

Embodiment 121: the method of any of embodiments 118-120, wherein the cancer is selected from the group consisting of a solid tumor, a hematologic cancer, bladder cancer, brain cancer, breast cancer, colon cancer, gastric cancer, glioma, head cancer, leukemia, liver cancer, lung cancer, lymphoma, myeloma, neck cancer, ovarian cancer, melanoma, pancreatic cancer, renal cancer, salivary cancer, stomach cancer, thymic epithelial cancer, and thyroid cancer.

Embodiment 122: the method of any of embodiments 118-121, wherein the cancer is small-cell lung cancer, non-small cell lung cancer, adenocarcinoma of the lung, or squamous carcinoma of the lung.

Embodiment 123: the method of any of embodiments 118-122, wherein the cancer is non-small cell lung cancer.

Embodiment 124: the method of any of embodiments 113-123, wherein the subject is a human.

Embodiment 125: the method of any of embodiments 113-124, wherein the pharmaceutical composition is administered topically, transdermally, subcutaneously, epicutaneously, intradermally, orally, sublingually, buccally, rectally, vaginally, intravenously, intraarterially, intramuscularly, intraosseously, intracardially, intraperitoneally, transmucosally, intravitreally, subretinally, suprachoroidally, intracranially, intrathecally, intraventricularly, intraarticularly, peri-articularly, intratumorally, locally, or via inhalation to the subject.

Embodiment 126: the method of any of embodiments 113-125, wherein the pharmaceutical composition is administered orally, intranasally, intratracheally, or via inhalation to the subject.

Embodiment 127: the method of any of embodiments 113-126, wherein the pharmaceutical composition is administered intratumorally to the subject.

Embodiment 128: the method of any of embodiments 113-127, wherein the pharmaceutical composition is administered intratracheally to the subject.

The specification is considered to be sufficient to enable one skilled in the art to practice the present disclosure. Various modifications of the present disclosure in addition to those shown and described herein will become apparent to those skilled in the art from the foregoing description and fall within the scope of the appended claims.

### EXAMPLES

The present disclosure will be more fully understood by reference to the following examples. It should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Construction of a Modified Herpes Simplex Virus Encoding Human IL-12 and IL-2

The following example describes the engineering of a recombinant herpes simplex virus type 1 (HSV-1) that successfully encoded human IL-12 and IL-2 and expressed full-length human IL-12 and IL-2 protein.

A recombinant HSV-1 was engineered to incorporate human IL12 and IL2 expression cassettes containing a heterologous promoter and polyA sequence. Viral plaques putatively containing the human IL-12 and IL-2 cassettes were picked and screened by infection in a complementing cell line to test for human IL-12 and IL-2 protein expression via western blot analysis (data not shown). High expressing clones termed HSV-IL12/IL2 were subsequently selected for additional in vitro analysis.

Human embryonic kidney (HEK) 293 FT cells were mock infected with vehicle control or were infected with HSV-IL12/IL2 at a multiplicity of infection (MOI) of 1 in serum-free cell culture medium. 24- or 48-hours post-infection, cell pellets were harvested, lysed in RIPA buffer containing protease inhibitors, and protein content was quantified via a BCA assay. 30-40 μg of each sample was loaded and run on a 4-20% acrylamide gel, and expression of the HSV-encoded human protein was assessed via western blot analysis (data not shown). Recombinant human IL-12 or IL-2 was loaded on the gel as a positive control. While no human IL-12 or IL-2 was detected in the uninfected control cells, robust expression of human IL-12 (data not shown) and IL-2 (data not shown) was observed after infection with HSV-IL12/IL2.

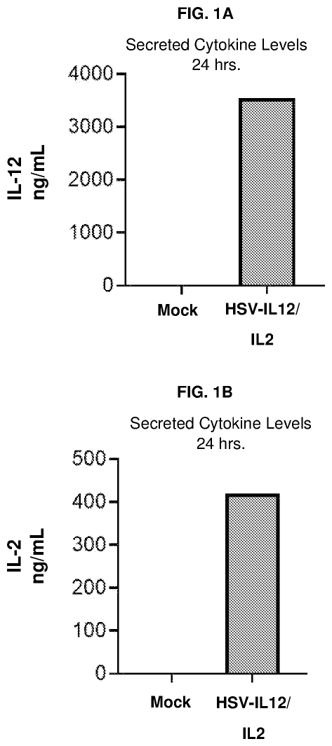

Because IL-12 and IL-2 are naturally secreted proteins, HEK293FT cell culture supernatants were also harvested and tested for the presence of the human protein by ELISA. In line with the western blot data, human IL-12 (over 3,000 ng/ml; FIG. 1A) and IL-2 (over 400 ng/ml; FIG. 1B) were detected in the supernatants of HEK293FT cells infected with HSV-IL12/IL2, at MOIs of 1, suggesting that the full-length human protein was being properly processed/secreted after expression from the recombinant vector.

Taken together, the data presented in this example indicated that a recombinant HSV-1 vector efficiently infected multiple cell types and was capable of expressing the human IL-12 and IL-2 transgenes encoded therein. Furthermore, the data indicated that the exogenous human proteins were subsequently (properly) secreted from infected cells.

### Example 2: In Vitro Murine IL-12 and IL-2 Bioactivity Assay

The following example describes the engineering of a recombinant herpes simplex virus type 1 (HSV-1) that successfully encoded murine IL-12 or IL-2 and expressed full-length murine IL-12 or IL-2 protein.

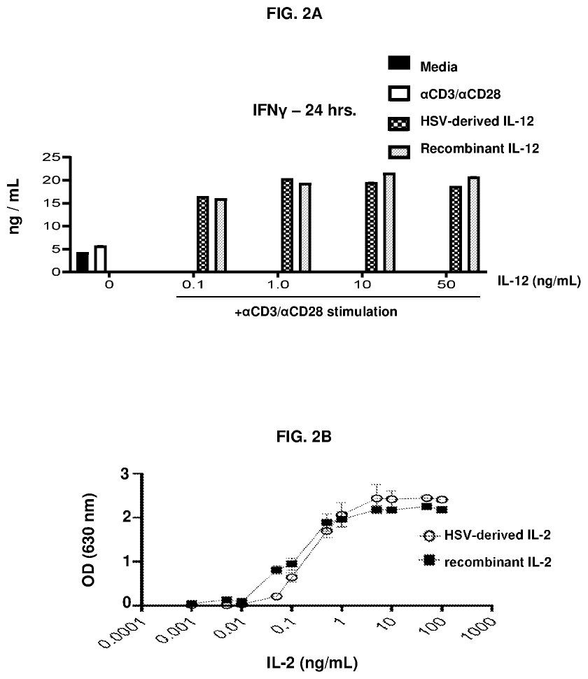

The objective of this study was, in part, to determine if the murine IL-12 protein and IL-2 proteins made from recombinant HSV-1 vectors were as bioactive as commercially available recombinant IL-12 and IL-2 protein. HEK293FT cells were infected with HSV-IL12 or HSV-IL2 at an MOI of 1 in serum-free cell culture medium for 24-hours. Cell culture supernatants were harvested and IL-12 and IL-2 concentrations were determined by ELISA. As indicated in FIGS. 2A-2B, serial dilutions of supernatants from HEK293FT infections with HSV-IL12/HSV-IL2 were added to murine BALB/c splenocytes (FIG. 2A) or HEK-Blue™-IL2 cells (FIG. 2B), to determine bioactivity of vector-derived IL-12 or IL-2, respectively. For splenocyte stimulations, commercially available murine αCD3/αCD28 coated beads were washed and added to cultures to induce stimulation and Interferon (IFN)γ secretion. Commercially available recombinant IL-12 protein (FIG. 2A) or IL-2 protein (FIG. 2B) was added to some cultures at similar concentrations for comparison. 24-hours post-stimulation, murine splenocyte or HEK-Blue™-IL2 cell culture supernatants were harvested and assayed by IFNγ ELISA (FIG. 2A) or for secreted embryonic alkaline phosphatase (SEAP) activity (FIG. 2B).

As shown in FIG. 2A, the addition of αCD3/αCD28 coated beads resulted in the release of IFNγ from murine splenocytes. In comparison, the addition of viral supernatant from HSV-IL12 to the αCD3/αCD28 stimulated splenocytes induced greater levels of IFNγ secretion when compared to αCD3/αCD28 stimulation alone. Further, the HSV-IL12 dependent release of IFNγ was comparable to that induced by recombinant IL-12. As shown in FIG. 2B, the HSV-IL2 dependent SEAP activity was comparable to that of recombinant IL-2 in HEK-Blue™ IL-2 reporter cells.

Taken together, these results suggested the IL-12 and IL-2 protein made from the recombinant herpes simplex viruses were as bioactive as commercially available recombinant IL-12 and IL-2 protein, respectively.

### Example 3: Intradermal Administration and In Vivo Evaluation of HSV-IL12/HSV-IL2 in Healthy Mice

As the above studies confirmed IL-12 and IL-2 transgene bioactivity, the next step was to test HSV-IL12/HSV-IL2 in vivo. The present study evaluated IL-12 and/or IL-2 expression, following a single dose (8×10⁸ PFU) of HSV-IL12/HSV-IL2 administered intradermally to C57BL/6 animals. IL-12 and IL-2 expression was assessed by nucleic acid and protein analysis in skin tissue at the indicated time points following vector administration. All procedures conducted were in compliance with applicable animal welfare acts and were approved by the local Institutional Animal Care and Use Committee (IACUC).

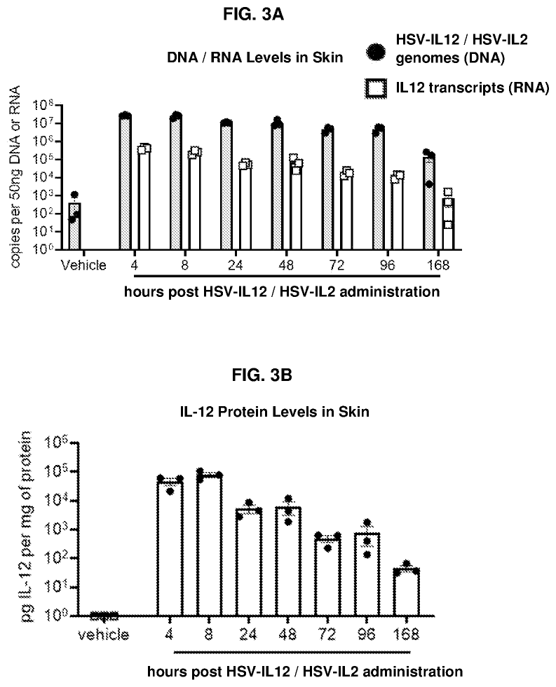

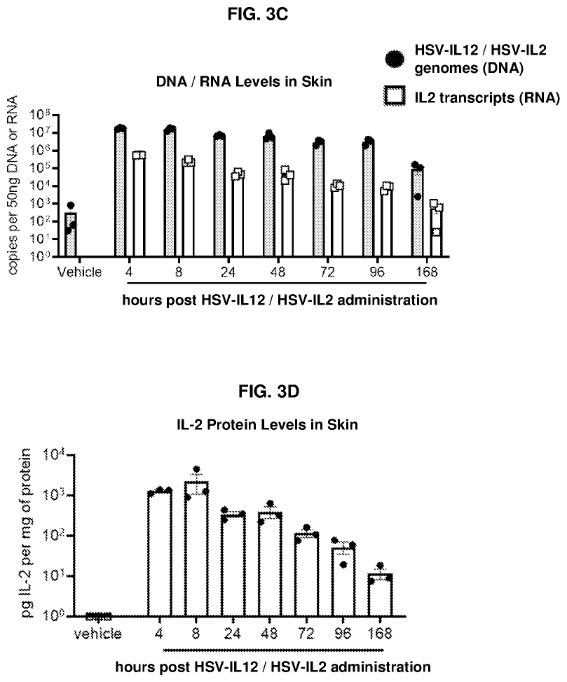

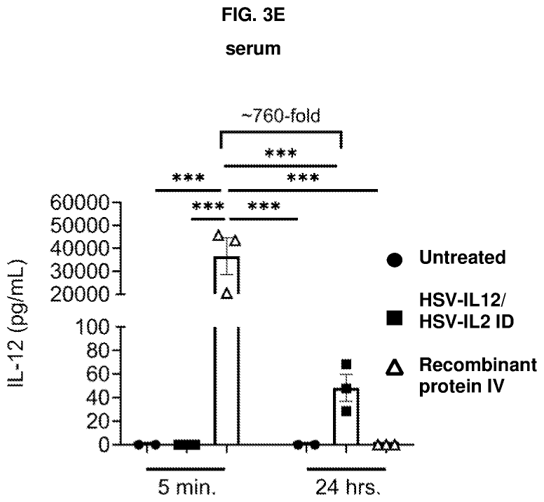

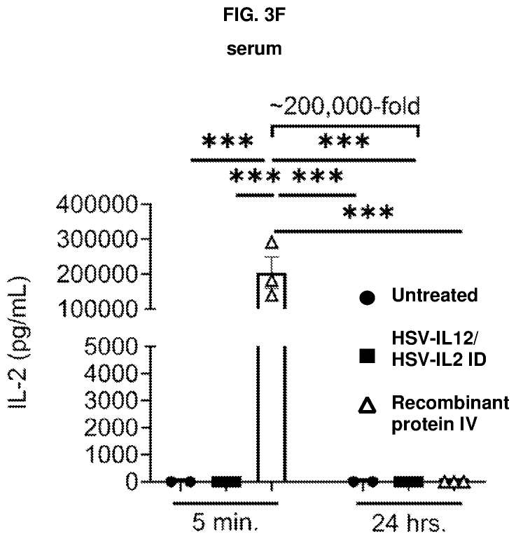

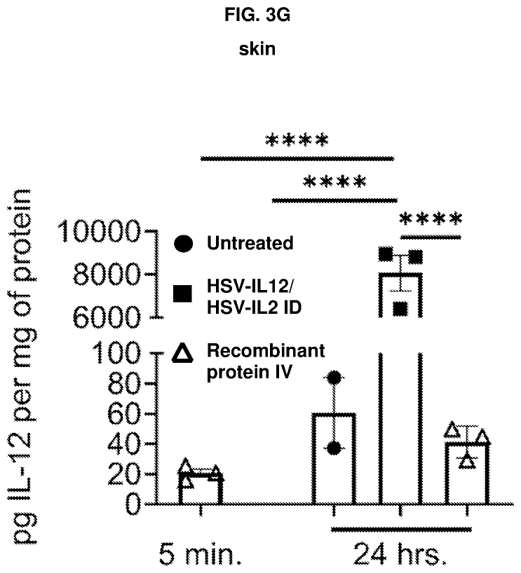

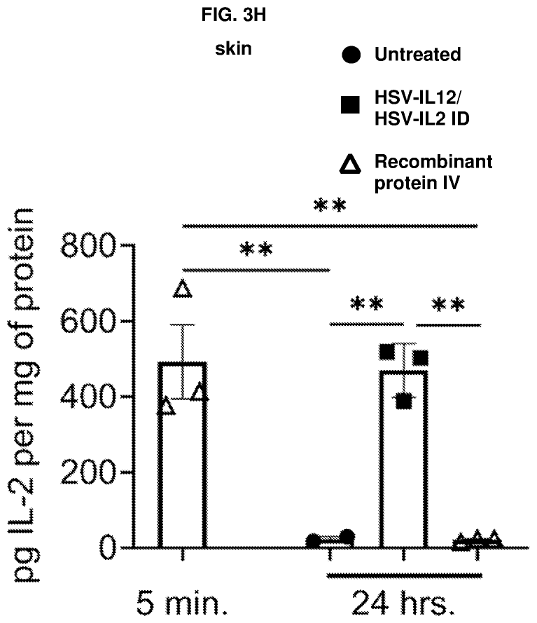

Post-sacrifice nucleic acid analysis of skin tissue indicated that IL12 (FIG. 3A) and IL2 (FIG. 3C) vector genomes and transcripts were detectable to at least 168-hours post-administration. In addition, skin biopsies were homogenized for IL-12 (FIG. 3B) and IL-2 (FIG. 3D) protein analysis by ELISA. Peak cytokine protein levels were observed at 8-hours post-injection, and these protein levels remained detectable through day 7 post-HSV-IL12/HSV-IL2 intradermal (ID) administration.

Another study was conducted to compare local effector concentrations in the skin and systemic cytokine exposure following ID administration of HSV-IL12/HSV-IL2 versus recombinant IL-12 and IL-2 proteins administered intravenously (FIGS. 3E-3H). C57BL/6 mice were treated with HSV-IL12/HSV-IL2 via ID injection (~10⁸ total PFU) or IL-12 and IL-2 recombinant protein (125 ng and 600 ng, respectively) intravenously at murine equivalent doses to human IL-12 and IL-2 that demonstrated clinical efficacy and toxicity in humans. Untreated animals served as negative controls. Serum samples and skin biopsies were taken at five minutes or 24-hours post-treatment for analysis by ELISA. As shown in FIGS. 3E-3H, there was minimal systemic cytokine exposure (FIGS. 3E-3F) following ID administration of HSV-IL12/HSV-IL2, coupled with robust expression of vector-derived IL-12 and IL-2 observed in skin biopsies, compared to intravenous (IV) recombinant protein therapy (FIGS. 3G-3H). Taken together, these data indicate vector-mediated intradermal delivery of IL-12 and IL-2 minimizes systemic cytokine exposure while enhancing local effector concentrations as compared to recombinant proteins administered intravenously at clinically-relevant doses.

### Example 4: Intratracheal Administration and In Vivo Evaluation of HSV-IL12/HSV-IL2 in Healthy Mice

The objective of this study was, in part, to evaluate transgene expression in the lungs following intratracheal (IT) administration of HSV-IL12/HSV-IL2.

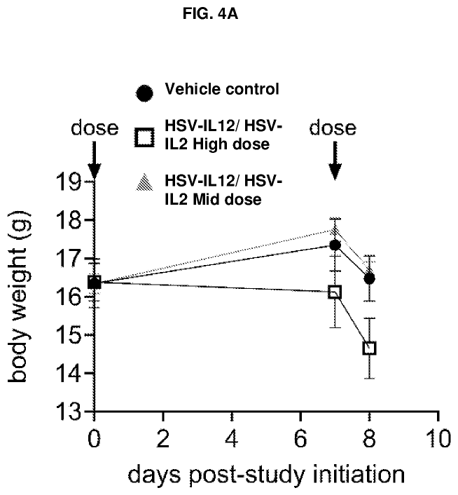

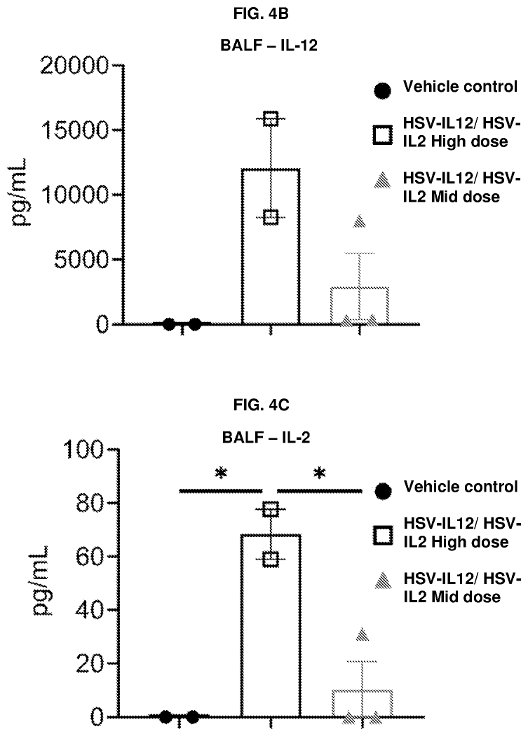

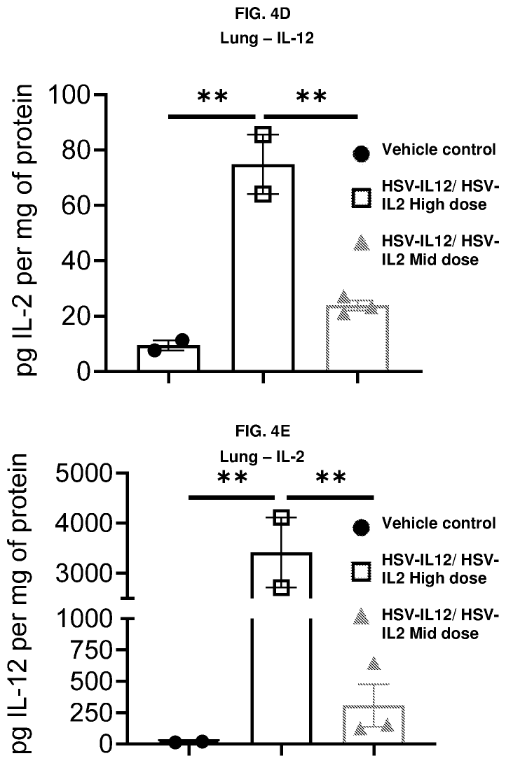

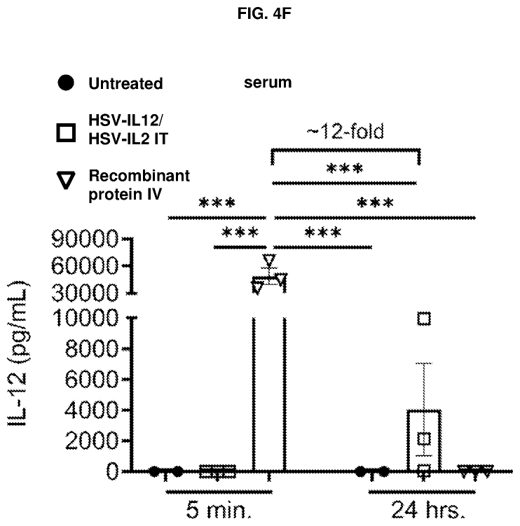

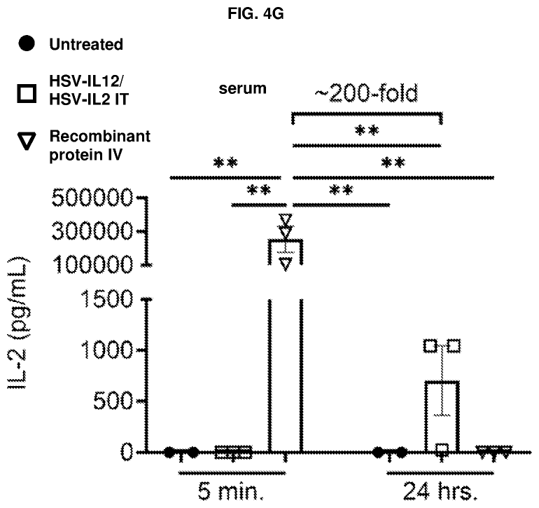

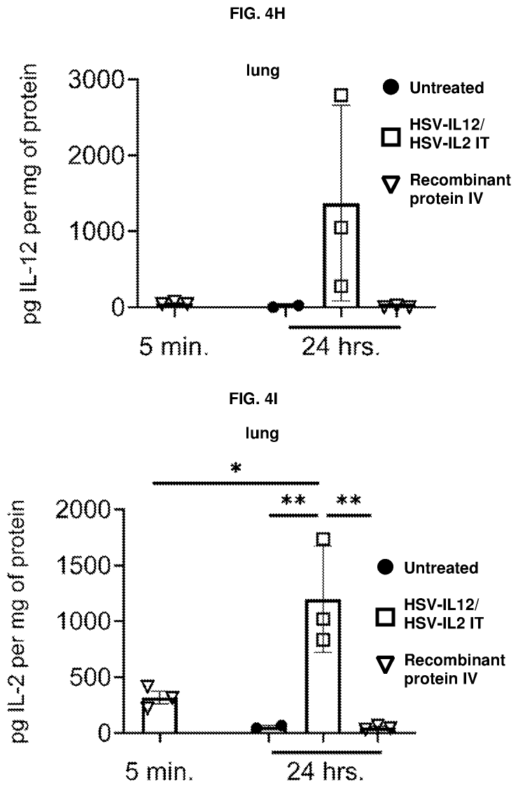

BALB/c animals were administered a single dose of HSV-IL12/HSV-IL2 intratracheally, at one of two doses (high or mid dose; ~10⁸ or ~10⁷ total PFU, respectively) on days 0 and 7 of the study. Animals were sacrificed on day 8, 24-hours following their last dose. Body weights were taken once weekly to assess toxicity, and IL-12 and IL-2 protein expression was assessed in lung tissue and bronchoalveolar lavage fluid (BALF) by ELISA. Results from this study indicate no significant differences in bodyweights between groups (FIG. 4A). With regards to effector expression, IL-12 and IL-2 protein levels in the BALF and lung tissue were significantly higher in animals treated with HSV-IL12/HSV-IL2 compared to vehicle control (FIGS. 4B-4D). Taken together, these data suggest HSV-IL12/HSV-IL2 administered intratracheally is well-tolerated and results in detectable and dose-dependent IL-12 and IL-2 expression in the lungs.

Next, a study was conducted to compare local effector concentrations in the lung and systemic cytokine exposure following IT administration of HSV-IL12/HSV-IL2 versus recombinant IL-12 and IL-2 proteins administered intravenously. BALB/c mice were treated with HSV-IL12/HSV-IL2 (~10⁷ total PFU) intratracheally or with IL-12 and IL-2 recombinant proteins (125 ng and 600 ng, respectively) intravenously at murine equivalent doses to human IL-12 and IL-2 that demonstrated clinical efficacy and toxicity in humans. Untreated animals served as negative controls. Serum samples and lungs were taken at five minutes or 24-hours post-treatment for analysis by ELISA (FIGS. 4F-41). As shown in FIGS. 4F-4/there was minimal systemic cytokine exposure (FIGS. 4F-4G) following administration of HSV-IL12/HSV-IL2 compared to IV recombinant protein treatment, in addition to more robust expression of IL-12 and IL-2 in lung homogenates (FIGS. 4H-4/). Taken together, these data indicate vector-mediated IT delivery of IL-12 and IL-2 minimizes systemic cytokine exposure while augmenting IL-12 and IL-2 concentrations in the lungs compared to recombinant proteins administered intravenously at clinically-relevant doses.

### Example 5: HSV-IL12/HSV-IL2 Efficacy in an In Vivo Murine Model of Melanoma

The objective of this study was, in part, to evaluate the efficacy of intradermal administration of HSV-IL12/HSV-IL2 therapy at inhibiting B16F10 melanoma tumor growth in the skin.

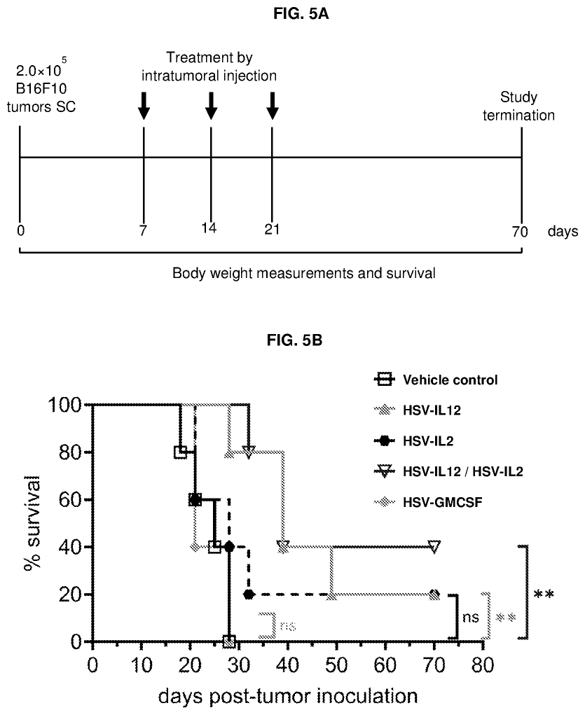

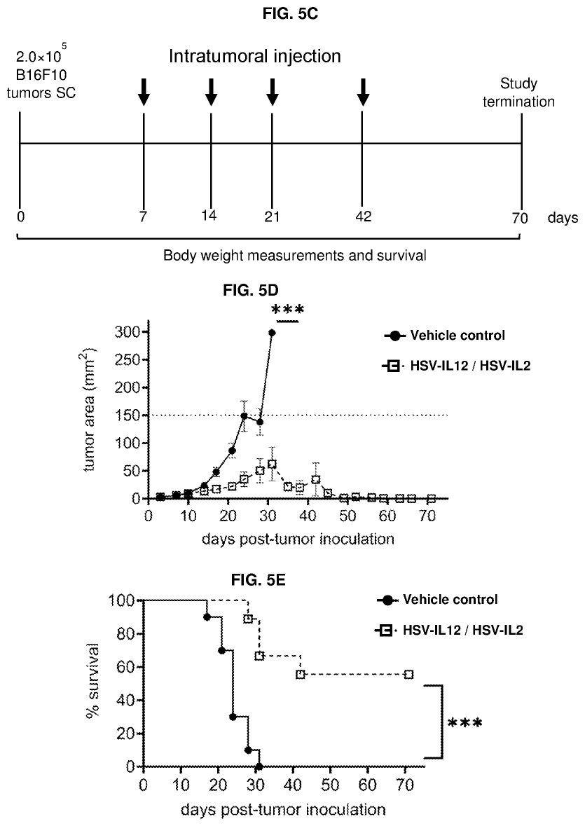

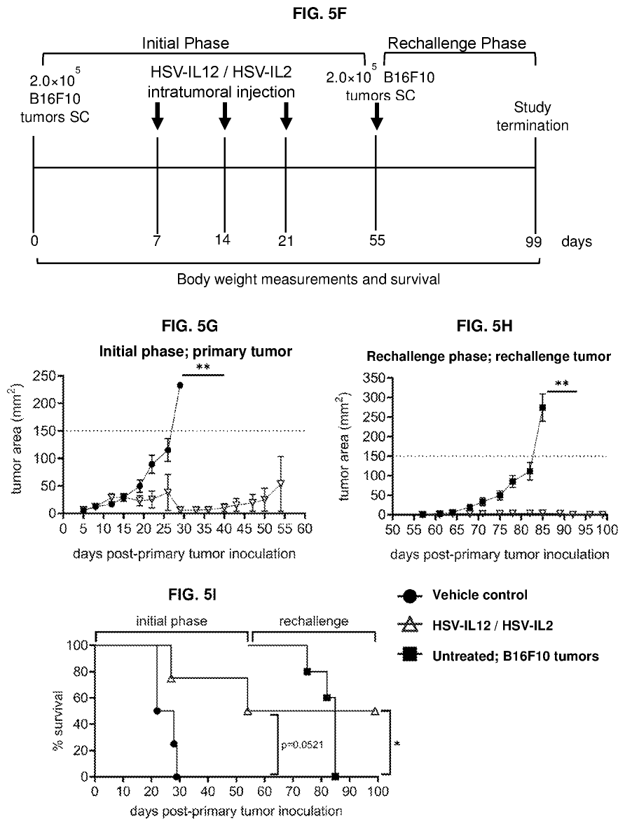

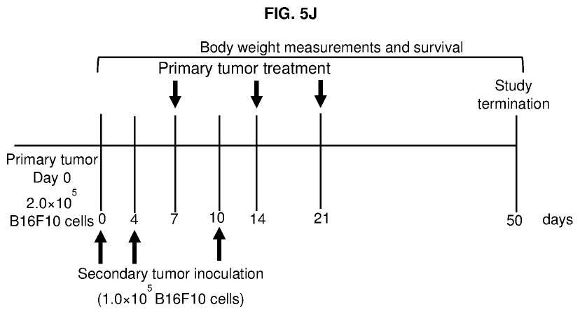

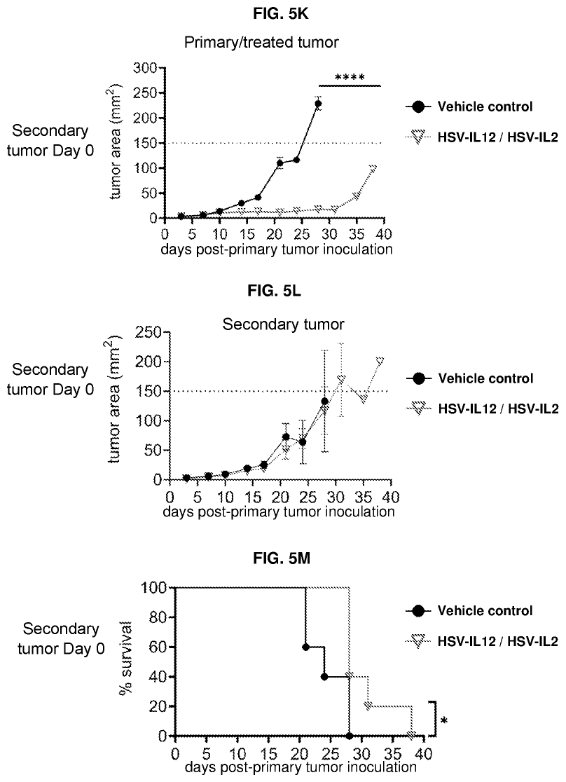

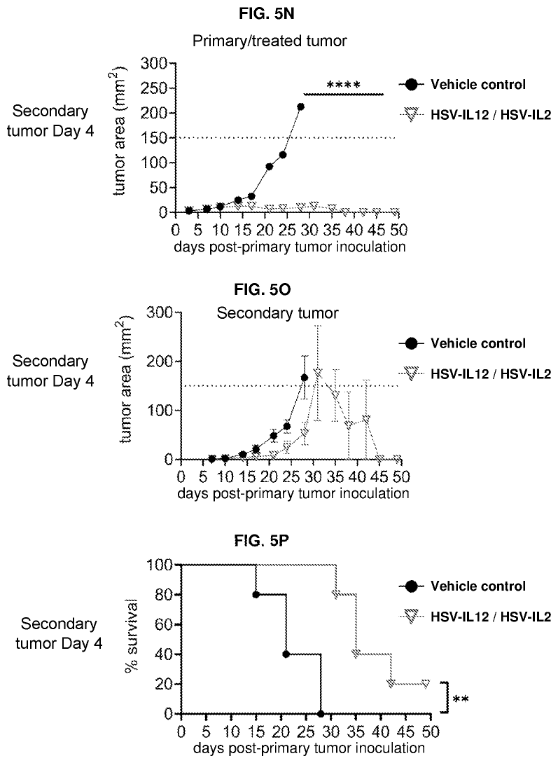

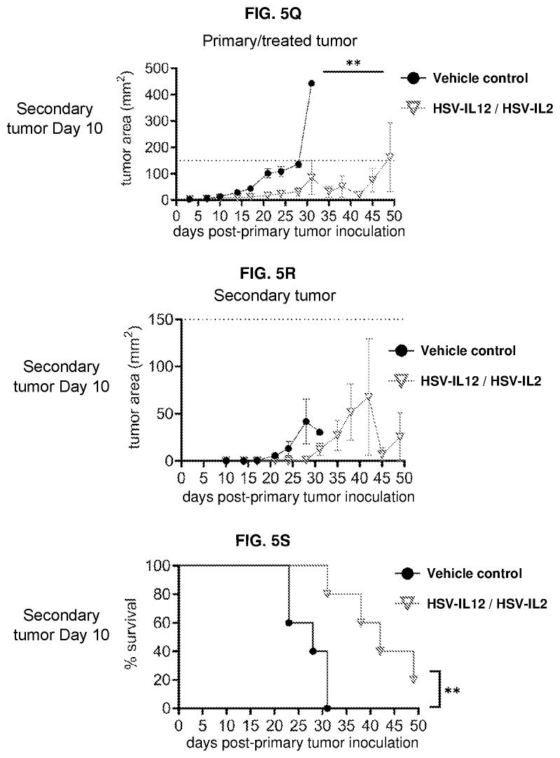

The B16F10 model of melanoma following subcutaneous (SC) administration is well established. As shown in FIG. 5A, animals were dosed (~10⁸ PFU) via intratumoral injection with the indicated vector at 7—, 14-, and 21-days following SC administration of B16F10 cells. As shown in FIG. 5B, the greatest survival benefit was observed in animals treated with the combination HSV-IL12/HSV-IL2 compared to groups treated with single vectors or vehicle control. Further, and as shown in FIGS. 5C-5E, this survival benefit (FIG. 5E) was potentiated when mice received a maintenance dose (FIG. 5C) of HSV-IL12/HSV-IL2 at day 42 post SC administration of B16F10 cells. Notably, tumor area (FIG. 5D) was significantly reduced in the HSV-IL12/HSV-IL2 treatment group compared to vehicle control. These data indicate HSV-IL12/HSV-IL2 intratumoral injection improves survival of B16F10 melanoma-bearing mice.

Next, a study was performed to assess whether HSV-IL12/HSV-IL2 treatment of primary B16F10 melanomas results in control of tumor recurrence (FIGS. 5F-5/). As shown in FIG. 5F, animals were dosed (~10⁸ PFU) via intratumoral injection with the indicated agent on days 7, 14, and 21 following SC administration of B16F10 cells (initial phase). Following this initial phase, an additional SC administration of B16F10 cells was given to surviving mice on day 55 following the initial dose of B16F10 tumors to mimic tumor recurrence (rechallenge phase). As shown in FIGS. 5G-5I, intratumoral administration of HSV-IL12/HSV-IL2 resulted in a significant reduction in tumor area compared to vehicle control or untreated tumors in both the initial phase (FIG. 5G) and the rechallenge phase (FIG. 5H), respectively. Notably, an HSV-IL12/HSV-IL2 dependent survival benefit was observed in both the initial and the rechallenge phases (FIG. 5/). Accordingly, this study suggests that HSV-IL12/HSV-IL2 treatment of primary B16F10 melanomas results in control of rechallenge melanoma tumors suggesting that vector-derived IL-12 and IL-2 treatment induces a durable anti-tumor memory response.

Next, a study was performed to determine whether HSV-IL12/HSV-IL2 treatment of a primary B16F10 melanoma results in an abscopal effect against a secondary B16F10 tumor (FIGS. 5J-5S). As shown in FIG. 5J, animals were dosed (~10⁸ PFU) via intratumoral injection with the indicated vector at 7-, 14-, and 21-days following SC administration of B16F10 cells (primary tumor). In addition, secondary tumor inoculation (1.0×10⁵ B16F10 cells) occurred either on day 0, 4, or 10 post-study initiation (FIG. 5J). Animals treated with HSV-IL12/HSV-IL2 displayed a reduction in primary tumor outgrowth, independent of secondary tumor administration (FIGS. 5K, 5N, and 5Q). Notably, an abscopal effect was observed with HSV-IL12/HSV-IL2 treatment in groups that received secondary tumors either on day 4 (FIG. 5O) or day 10 (FIG. 5R) post-study initiation; no effect was demonstrated in day 0 secondary tumor recipients (FIG. 5L). Overall, a survival benefit in all groups was observed in mice receiving HSV-IL12/HSV-IL2 treatment compared to vehicle control (FIGS. 5M, 5P, and 5S). Taken together, these data suggest HSV-IL12/HSV-IL2 treatment of primary melanoma tumors results in an abscopal effect against a secondary melanoma tumor.

### Example 6: HSV-IL12/HSV-IL2 Efficacy in an In Vivo Murine Model of Osteosarcoma

The objective of this study was, in part, to evaluate the efficacy of intratracheal administration of HSV-IL12/HSV-IL2 therapy at inhibiting metastatic osteosarcoma lung tumor growth.

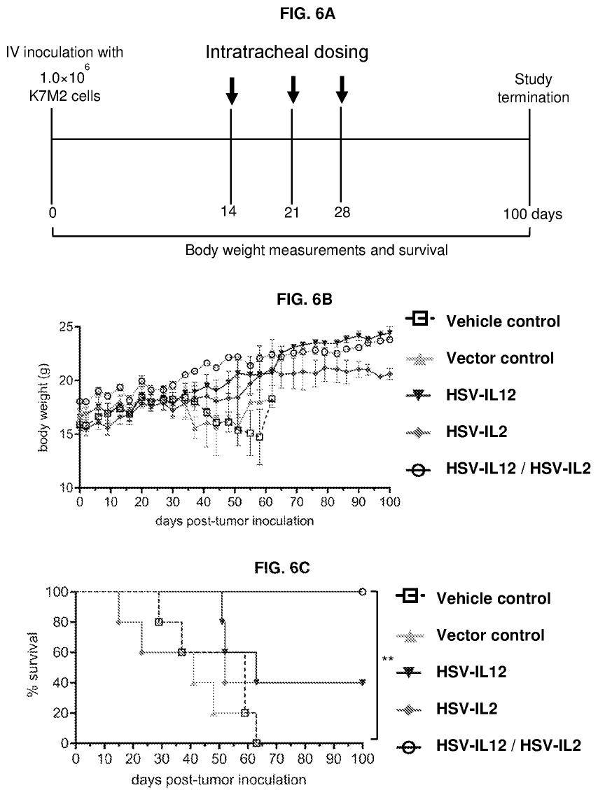

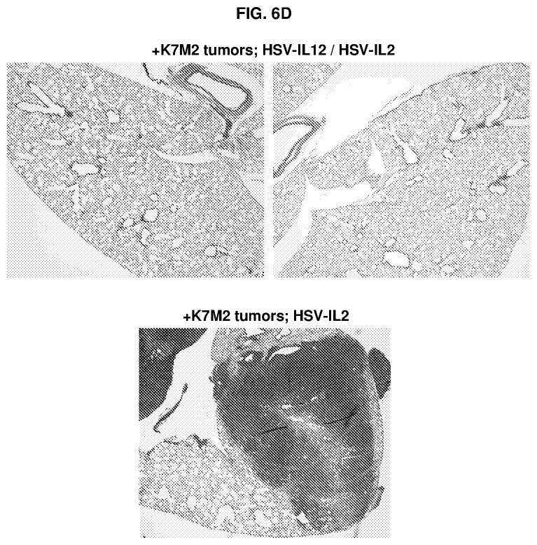

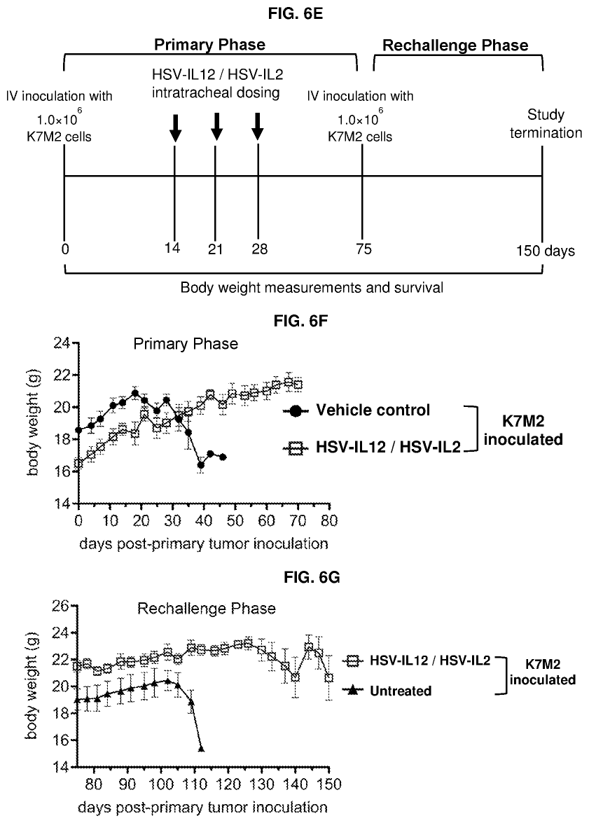

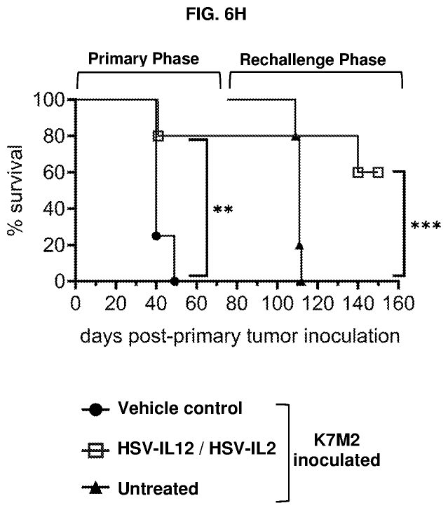

The K7M2 BALB/c model of osteosarcoma lung metastasis following intravenous (IV) administration is well established. As shown in FIG. 6A, animals were dosed (~10⁷ total PFU) via IT administration with the indicated vector at 14, 21, and 28 days following IV administration of K7M2 cells. Body weights for animals that received cytokine-expressing vectors were higher compared to those of vehicle and vector control (FIG. 6B) recipient animals. With respect to survival, a survival benefit was observed with single agent HSV-IL12 or HSV-IL2 treatment. Unexpectedly, combinatorial therapy with HSV-encoded IL-12 and IL-2 resulted in a synergistic effect with 100% survival of this group of animals for 100 days post-tumor instillation. (FIG. 6C). Evidence of tumor regression in the lung was also observed histologically (FIG. 6D). Taken together, these data indicate combinatorial HSV-IL12/HSV-IL2 administered intratracheally enhances tumor regression and survival in the K7M2 osteosarcoma lung metastasis model compared to control or single vector treatment.

Next, an additional study was performed to address whether IT administration of HSV-IL12/HSV-IL2 could minimize initial lung tumor outgrowth and delay tumor recurrence without additional therapeutic intervention. As shown in FIG. 6E, animals were dosed (~10⁷ total PFU) with the indicated vectors at 14, 21, and 28 days following IV administration of K7M2 cells (primary phase). At day 75, surviving animals received a second inoculation of K7M2 cells in the absence of any additional therapeutic intervention (rechallenge phase). As shown in FIGS. 6F-6G, animal body weight, in both the primary phase and rechallenge phase, was higher in HSV-IL12/HSV-IL2 treated animals compared to controls. Notably, the survival benefit observed in the HSV-IL12/HSV-IL2 group was maintained in the rechallenge phase (FIG. 6H). Taken together, these data indicate IT administration of HSV-IL12/HSV-IL2 not only minimizes initial K7M2 lung tumor outgrowth, but also delays tumor recurrence in the absence of additional therapeutic intervention.

### Example 7: A Recombinant Nucleic Acid as a Linear DNA Encoding IL-12 and IL-2

A first recombinant nucleic acid is engineered as a linear DNA to include a recombinant polynucleotide encoding a human IL-12. A second recombinant nucleic acid is engineered as a linear DNA to include a recombinant polynucleotide encoding a human IL-2. The recombinant nucleic acids are tested for their in vitro and/or in vivo expression of IL-12 and/or IL-2, as well as their efficacy as a cancer therapeutic. These results are not available at the time of filing.

A recombinant nucleic acid is engineered as a linear DNA to include a recombinant polynucleotide encoding a human IL-12 and a recombinant polynucleotide encoding a human IL-2. The recombinant nucleic acid is tested for its in vitro and/or in vivo expression of IL-12 and/or IL-2, as well as its efficacy as a cancer therapeutic. These results are not available at the time of filing.

### Example 8: A Recombinant Nucleic Acid as a Circular DNA Encoding IL-12 and IL-2

A first recombinant nucleic acid is engineered as a circular DNA to include a recombinant polynucleotide encoding a human IL-12. A second recombinant nucleic acid is engineered as a circular DNA to include a recombinant polynucleotide encoding a human IL-2. The recombinant nucleic acids are tested for their in vitro and/or in vivo expression of IL-12 and/or IL-2, as well as their efficacy as a cancer therapeutic. These results are not available at the time of filing.

A recombinant nucleic acid is engineered as a circular DNA to include a recombinant polynucleotide encoding a human IL-12 and a recombinant polynucleotide encoding a human IL-2. The recombinant nucleic acid is tested for its in vitro and/or in vivo expression of IL-12 and/or IL-2, as well as its efficacy as a cancer therapeutic. These results are not available at the time of filing.

### Example 9: A Recombinant Nucleic Acid as a Close Ended DNA Encoding IL-12 and IL-2

A first recombinant nucleic acid is engineered as a close ended DNA to include a recombinant polynucleotide encoding a human IL-12. A second recombinant nucleic acid is engineered as a close ended DNA to include a recombinant polynucleotide encoding a human IL-2. The recombinant nucleic acids are tested for their in vitro and/or in vivo expression of IL-12 and/or IL-2, as well as their efficacy as a cancer therapeutic. These results are not available at the time of filing.

A recombinant nucleic acid is engineered as a close ended DNA to include a recombinant polynucleotide encoding a human IL-12 and a recombinant polynucleotide encoding a human IL-2. The recombinant nucleic acid is tested for its in vitro and/or in vivo expression of IL-12 and/or IL-2, as well as its efficacy as a cancer therapeutic. These results are not available at the time of filing.

### Example 10: A Recombinant Nucleic Acid as an RNA Encoding IL-12 and IL-2

A first recombinant nucleic acid is engineered as an RNA to include a recombinant polynucleotide encoding a human IL-12. A second recombinant nucleic acid is engineered as an RNA to include a recombinant polynucleotide encoding a human IL-2. The recombinant nucleic acids are tested for their in vitro and/or in vivo expression of IL-12 and/or IL-2, as well as their efficacy as a cancer therapeutic. These results are not available at the time of filing.

A recombinant nucleic acid is engineered as an RNA to include a recombinant polynucleotide encoding a human IL-12 and a recombinant polynucleotide encoding a human IL-2. The recombinant nucleic acid is tested for its in vitro and/or in vivo expression of IL-12 and/or IL-2, as well as its efficacy as a cancer therapeutic. These results are not available at the time of filing.

### Example 11: A Recombinant Nucleic Acid as an mRNA Encoding IL-12 and IL-2

A first recombinant nucleic acid is engineered as an mRNA to include a recombinant polynucleotide encoding a human IL-12. A second recombinant nucleic acid is engineered as an mRNA to include a recombinant polynucleotide encoding a human IL-2. The recombinant nucleic acids are tested for their in vitro and/or in vivo expression of IL-12 and/or IL-2, as well as their efficacy as a cancer therapeutic. These results are not available at the time of filing.

A recombinant nucleic acid is engineered as an mRNA to include a recombinant polynucleotide encoding a human IL-12 and a recombinant polynucleotide encoding a human IL-2. The recombinant nucleic acid is tested for its in vitro and/or in vivo expression of IL-12 and/or IL-2, as well as its efficacy as a cancer therapeutic. These results are not available at the time of filing.

### Example 12: A Recombinant Nucleic Acid as an mmRNA Encoding IL-12 and IL-2

A first recombinant nucleic acid is engineered as an mmRNA to include a recombinant polynucleotide encoding a human IL-12. A second recombinant nucleic acid is engineered as an mmRNA to include a recombinant polynucleotide encoding a human IL-2. The recombinant nucleic acids are tested for their in vitro and/or in vivo expression of IL-12 and/or IL-2, as well as their efficacy as a cancer therapeutic. These results are not available at the time of filing.

A recombinant nucleic acid is engineered as an mmRNA to include a recombinant polynucleotide encoding a human IL-12 and a recombinant polynucleotide encoding a human IL-2. The recombinant nucleic acid is tested for its in vitro and/or in vivo expression of IL-12 and/or IL-2, as well as its efficacy as a cancer therapeutic. These results are not available at the time of filing.

### Example 13: A Recombinant Nucleic Acid as Described in any One or More of Examples 7-12 in a Recombinant Adenovirus Genome

A recombinant nucleic acid is engineered as described in any one or more of examples 7-12 in a recombinant adenovirus genome, optionally in an adenovirus gene delivery vehicle, and is tested for its in vitro and/or in vivo expression of IL-12 and/or IL-2, as well as its efficacy as a cancer therapeutic. These results are not available at the time of filing.

### Example 14: A Recombinant Nucleic Acid as Described in any One or More of Examples 7-12 in a Recombinant Retrovirus Genome

A recombinant nucleic acid is engineered as described in any one or more of examples 7-12 in a recombinant retrovirus genome, optionally in a retrovirus gene delivery vehicle, and is tested for its in vitro and/or in vivo expression of IL-12 and/or IL-2, as well as its efficacy as a cancer therapeutic. These results are not available at the time of filing.

### Example 15: A Recombinant Nucleic Acid as Described in any One or More of Examples 7-12 in a Recombinant Adeno-Associated Virus Genome

A recombinant nucleic acid is engineered as described in any one or more of examples 7-12 in a recombinant adeno-associated virus genome, optionally in an adeno-associated virus gene delivery vehicle, and is tested for its in vitro and/or in vivo expression of IL-12 and/or IL-2, as well as its efficacy as a cancer therapeutic. These results are not available at the time of filing.

### Example 16: A Recombinant Nucleic Acid as Described in any One or More of Examples 7-12 in a Recombinant Poxvirus Virus Genome

A recombinant nucleic acid is engineered as described in any one or more of examples 7-12 in a recombinant poxvirus genome, optionally in a poxvirus gene delivery vehicle, and is tested for its in vitro and/or in vivo expression of IL-12 and/or IL-2, as well as its efficacy as a cancer therapeutic. These results are not available at the time of filing.

### Example 17: A Recombinant Nucleic Acid as Described in any One or More of Examples 7-12 in a Recombinant Bacteriophage Genome

A recombinant nucleic acid is engineered as described in any one or more of examples 7-12 in a recombinant bacteriophage genome, optionally in a bacteriophage gene delivery vehicle, and is tested for its in vitro and/or in vivo expression of IL-12 and/or IL-2, as well as its efficacy as a cancer therapeutic. These results are not available at the time of filing.

### Example 18: A Recombinant Nucleic Acid as Described in any One or More of Examples 7-12 in a Recombinant Alphavirus Genome

A recombinant nucleic acid is engineered as described in any one or more of examples 7-12 in a recombinant alphavirus genome, optionally in an alphavirus gene delivery vehicle, and is tested for its in vitro and/or in vivo expression of IL-12 and/or IL-2, as well as its efficacy as a cancer therapeutic. These results are not available at the time of filing.

### Example 19: A Recombinant Nucleic Acid as Described in any One or More of Examples 7-12 in a Recombinant Picornavirus Genome

A recombinant nucleic acid is engineered as described in any one or more of examples 7-12 in a recombinant picornavirus genome, optionally in a picornavirus gene delivery vehicle, and is tested for its in vitro and/or in vivo expression of IL-12 and/or IL-2, as well as its efficacy as a cancer therapeutic. These results are not available at the time of filing.

### Example 20: A Recombinant Nucleic Acid as Described in any One or More of Examples 7-12 in a Recombinant Iridovirus Genome

A recombinant nucleic acid is engineered as described in any one or more of examples 7-12 in a recombinant iridovirus genome, optionally in an iridovirus gene delivery vehicle, and is tested for its in vitro and/or in vivo expression of IL-12 and/or IL-2, as well as its efficacy as a cancer therapeutic. These results are not available at the time of filing.

### Example 21: A Recombinant Nucleic Acid as Described in any One or More of Examples 7-12 in a Recombinant Newcastle Disease Virus Genome

A recombinant nucleic acid is engineered as described in any one or more of examples 7-12 in a recombinant Newcastle disease virus genome, optionally in a Newcastle disease virus gene delivery vehicle, and is tested for its in vitro and/or in vivo expression of IL-12 and/or IL-2, as well as its efficacy as a cancer therapeutic. These results are not available at the time of filing.

### Example 22: A Recombinant Nucleic Acid as Described in any One or More of Examples 7-12 in a Recombinant Baculovirus Genome

A recombinant nucleic acid is engineered as described in any one or more of examples 7-12 in a recombinant baculovirus genome, optionally in a baculovirus gene delivery vehicle, and is tested for its in vitro and/or in vivo expression of IL-12 and/or IL-2, as well as its efficacy as a cancer therapeutic. These results are not available at the time of filing.

### Example 23: A Recombinant Nucleic Acid as Described in any One or More of Examples 7-12 in a Recombinant Geminivirus Genome

A recombinant nucleic acid is engineered as described in any one or more of examples 7-12 in a recombinant geminivirus genome, optionally in a geminivirus gene delivery vehicle, and is tested for its in vitro and/or in vivo expression of IL-12 and/or IL-2, as well as its efficacy as a cancer therapeutic. These results are not available at the time of filing.

### Example 24: A Recombinant Nucleic Acid as Described in any One or More of Examples 7-12 in a Recombinant Caulimovirus Genome

A recombinant nucleic acid is engineered as described in any one or more of examples 7-12 in a recombinant caulimovirus genome, optionally in a caulimovirus gene delivery vehicle, and is tested for its in vitro and/or in vivo expression of IL-12 and/or IL-2, as well as its efficacy as a cancer therapeutic. These results are not available at the time of filing.

### Example 25: A Recombinant Nucleic Acid as Described in any One or More of Examples 7-12 in a Recombinant Anellovirus Genome

A recombinant nucleic acid is engineered as described in any one or more of examples 7-12 in a recombinant anellovirus genome, optionally in an anellovirus gene delivery vehicle, and is tested for its in vitro and/or in vivo expression of IL-12 and/or IL-2, as well as its efficacy as a cancer therapeutic. These results are not available at the time of filing.

### Example 26: A Recombinant Nucleic Acid as Described in any One or More of Examples 7-12 in One or More Chemical Compounds, Bacteria, Mammalian Cells, and/or Physical Delivery System

A recombinant nucleic acid is engineered as described in any one or more of examples 7-12 in one or more chemical compounds, bacteria, mammalian cells, and/or physical delivery systems as a non-viral gene delivery vehicle. The one or more chemical compounds, bacteria, mammalian cells, and/or physical delivery systems are tested for its in vitro and/or in vivo expression of IL-12 and/or IL-2, as well as its efficacy as a cancer therapeutic. These results are not available at the time of filing.

### Example 27: A Recombinant Nucleic Acid as Described in any One or More of Examples 7-12 in One or More Polymer Compounds, Lipid Compounds, and/or Inorganic Compounds

A recombinant nucleic acid is engineered as described in any one or more of examples 7-12 in one or more polymer compounds, lipid compounds, and/or inorganic compounds as a non-viral gene delivery vehicle. The one or more polymer compounds, lipid compounds, and/or inorganic compounds are tested for its in vitro and/or in vivo expression of IL-12 and/or IL-2, as well as its efficacy as a cancer therapeutic. These results are not available at the time of filing.

### Example 28: a recombinant nucleic acid as described in any one or more of examples 7-12 in one or more natural polymers, synthetic polymers, biopolymers, biodegradable polymers, cationic polymers, protein polymers, and/or polysaccharide polymers

A recombinant nucleic acid is engineered as described in any one or more of examples 7-12 in one or more natural polymers, synthetic polymers, biopolymers, biodegradable polymers, cationic polymers, protein polymers, and/or polysaccharide polymers as a non-viral gene delivery vehicle. The one or more natural polymers, synthetic polymers, biopolymers, biodegradable polymers, cationic polymers, protein polymers, and/or polysaccharide polymers are tested for its in vitro and/or in vivo expression of IL-12 and/or IL-2, as well as its efficacy as a cancer therapeutic. These results are not available at the time of filing.

### Example 29: A Recombinant Nucleic Acid as Described in any One or More of Examples 7-12 in One or More Natural Polymers

A recombinant nucleic acid is engineered as described in any one or more of examples 7-12 in one or more natural polymers as a non-viral gene delivery vehicle. The one or more natural polymers are tested for its in vitro and/or in vivo expression of IL-12 and/or IL-2, as well as its efficacy as a cancer therapeutic. These results are not available at the time of filing.

### Example 30: A Recombinant Nucleic Acid as Described in any One or More of Examples 7-12 in One or More Synthetic Polymers

A recombinant nucleic acid is engineered as described in any one or more of examples 7-12 in one or more synthetic polymers as a non-viral gene delivery vehicle. The one or more synthetic polymers are tested for its in vitro and/or in vivo expression of IL-12 and/or IL-2, as well as its efficacy as a cancer therapeutic. These results are not available at the time of filing.

### Example 31: A Recombinant Nucleic Acid as Described in any One or More of Examples 7-12 in One or More Biopolymers

A recombinant nucleic acid is engineered as described in any one or more of examples 7-12 in one or more biopolymers as a non-viral gene delivery vehicle. The one or more biopolymers are tested for its in vitro and/or in vivo expression of IL-12 and/or IL-2, as well as its efficacy as a cancer therapeutic. These results are not available at the time of filing.

### Example 32: A Recombinant Nucleic Acid as Described in any One or More of Examples 7-12 in One or More Biodegradable Polymers

A recombinant nucleic acid is engineered as described in any one or more of examples 7-12 in one or more biodegradable polymers as a non-viral gene delivery vehicle. The one or more biodegradable polymers are tested for its in vitro and/or in vivo expression of IL-12 and/or IL-2, as well as its efficacy as a cancer therapeutic. These results are not available at the time of filing.

### Example 33: A Recombinant Nucleic Acid as Described in any One or More of Examples 7-12 in One or More Cationic Polymers

A recombinant nucleic acid is engineered as described in any one or more of examples 7-12 in one or more cationic polymers as a non-viral gene delivery vehicle. The one or more cationic polymers are tested for its in vitro and/or in vivo expression of IL-12 and/or IL-2, as well as its efficacy as a cancer therapeutic. These results are not available at the time of filing.

### Example 34: A Recombinant Nucleic Acid as Described in any One or More of Examples 7-12 in One or More Protein Polymers

A recombinant nucleic acid is engineered as described in any one or more of examples 7-12 in one or more protein polymers as a non-viral gene delivery vehicle. The one or more protein polymers are tested for its in vitro and/or in vivo expression of IL-12 and/or IL-2, as well as its efficacy as a cancer therapeutic. These results are not available at the time of filing.

### Example 35: A Recombinant Nucleic Acid as Described in any One or More of Examples 7-12 in One or More Polysaccharide Polymer

A recombinant nucleic acid is engineered as described in any one or more of examples 7-12 in one or more polysaccharide polymers as a non-viral gene delivery vehicle. The one or more polysaccharide polymers are tested for its in vitro and/or in vivo expression of IL-12 and/or IL-2, as well as its efficacy as a cancer therapeutic. These results are not available at the time of filing.

### Example 36: A Recombinant Nucleic Acid as Described in any One or More of Examples 7-12 in One or More Nanoparticles, Lipid Nanoparticles, Liposomes, Cationic Liposome, Solid Lipid Nanoparticle, Lipid Emulsions, Lipidoids, Cytofectins, Surfactants, and/or Gemini Surfactants

A recombinant nucleic acid is engineered as described in any one or more of examples 7-12 in one or more nanoparticles, lipid nanoparticles, liposomes, cationic liposome, solid lipid nanoparticle, lipid emulsions, lipidoids, cytofectins, surfactants, and/or gemini surfactants as a non-viral gene delivery vehicle. The one or more nanoparticles, lipid nanoparticles, liposomes, cationic liposome, solid lipid nanoparticle, lipid emulsions, lipidoids, cytofectins, surfactants, and/or gemini surfactants are tested for its in vitro and/or in vivo expression of IL-12 and/or IL-2, as well as its efficacy as a cancer therapeutic. These results are not available at the time of filing.

### Example 37: A Recombinant Nucleic Acid as Described in any One or More of Examples 7-12 in One or More Nanoparticles

A recombinant nucleic acid is engineered as described in any one or more of examples 7-12 in one or more nanoparticles as a non-viral gene delivery vehicle. The one or more nanoparticles are tested for its in vitro and/or in vivo expression of IL-12 and/or IL-2, as well as its efficacy as a cancer therapeutic. These results are not available at the time of filing.

### Example 38: A Recombinant Nucleic Acid as Described in any One or More of Examples 7-12 in One or More Lipid Nanoparticles

A recombinant nucleic acid is engineered as described in any one or more of examples 7-12 in one or more lipid nanoparticles as a non-viral gene delivery vehicle. The one or more lipid nanoparticles are tested for its in vitro and/or in vivo expression of IL-12 and/or IL-2, as well as its efficacy as a cancer therapeutic. These results are not available at the time of filing.

### Example 39: A Recombinant Nucleic Acid as Described in any One or More of Examples 7-12 in One or More Liposomes

A recombinant nucleic acid is engineered as described in any one or more of examples 7-12 in one or more liposomes as a non-viral gene delivery vehicle. The one or more liposomes are tested for its in vitro and/or in vivo expression of IL-12 and/or IL-2, as well as its efficacy as a cancer therapeutic. These results are not available at the time of filing.

### Example 40: A Recombinant Nucleic Acid as Described in any One or More of Examples 7-12 in One or More Cationic Liposomes

A recombinant nucleic acid is engineered as described in any one or more of examples 7-12 in one or more cationic liposomes as a non-viral gene delivery vehicle. The one or more cationic liposomes are tested for its in vitro and/or in vivo expression of IL-12 and/or IL-2, as well as its efficacy as a cancer therapeutic. These results are not available at the time of filing.

### Example 41: A Recombinant Nucleic Acid as Described in any One or More of Examples 7-12 in One or More Solid Lipid Nanoparticles

A recombinant nucleic acid is engineered as described in any one or more of examples 7-12 in one or more solid lipid nanoparticles as a non-viral gene delivery vehicle. The one or more solid lipid nanoparticles are tested for its in vitro and/or in vivo expression of IL-12 and/or IL-2, as well as its efficacy as a cancer therapeutic. These results are not available at the time of filing.

### Example 42: A Recombinant Nucleic Acid as Described in any One or More of Examples 7-12 in One or More Lipid Emulsions

A recombinant nucleic acid is engineered as described in any one or more of examples 7-12 in one or more lipid emulsions as a non-viral gene delivery vehicle. The one or more lipid emulsions are tested for its in vitro and/or in vivo expression of IL-12 and/or IL-2, as well as its efficacy as a cancer therapeutic. These results are not available at the time of filing.

### Example 43: A Recombinant Nucleic Acid as Described in any One or More of Examples 7-12 in One or More Lipidoids

A recombinant nucleic acid is engineered as described in any one or more of examples 7-12 in one or more lipidoids as a non-viral gene delivery vehicle. The one or more lipidoids are tested for its in vitro and/or in vivo expression of IL-12 and/or IL-2, as well as its efficacy as a cancer therapeutic. These results are not available at the time of filing.

### Example 44: A Recombinant Nucleic Acid as Described in any One or More of Examples 7-12 in One or More Cytofectins

A recombinant nucleic acid is engineered as described in any one or more of examples 7-12 in one or more cytofectins as a non-viral gene delivery vehicle. The one or more cytofectins are tested for its in vitro and/or in vivo expression of IL-12 and/or IL-2, as well as its efficacy as a cancer therapeutic. These results are not available at the time of filing.

### Example 45: A Recombinant Nucleic Acid as Described in any One or More of Examples 7-12 in One or More Surfactants

A recombinant nucleic acid is engineered as described in any one or more of examples 7-12 in one or more surfactants as a non-viral gene delivery vehicle. The one or more surfactants are tested for its in vitro and/or in vivo expression of IL-12 and/or IL-2, as well as its efficacy as a cancer therapeutic. These results are not available at the time of filing.

### Example 46: A Recombinant Nucleic Acid as Described in any One or More of Examples 7-12 in One or More Gemini Surfactants

A recombinant nucleic acid is engineered as described in any one or more of examples 7-12 in one or more gemini surfactants as a non-viral gene delivery vehicle. The one or more gemini surfactants are tested for its in vitro and/or in vivo expression of IL-12 and/or IL-2, as well as its efficacy as a cancer therapeutic. These results are not available at the time of filing.

### Example 47: A Recombinant Nucleic Acid as Described in any One or More of Examples 7-12 in One or More Nanocarriers, DNA Nanclews, Gold Nanoparticles, Carbon Nanotubes, Graphenes, Quantum Dots, Up-Conversion Nanoparticles, Silica Nanoparticles, Iron Oxides, and/or Ferritins

A recombinant nucleic acid is engineered as described in any one or more of examples 7-12 in one or more nanocarriers, DNA nanclews, gold nanoparticles, carbon nanotubes, graphenes, quantum dots, up-conversion nanoparticles, silica nanoparticles, iron oxides, and/or ferritins as a non-viral gene delivery vehicle. The one or more nanocarriers, DNA nanclews, gold nanoparticles, carbon nanotubes, graphenes, quantum dots, up-conversion nanoparticles, silica nanoparticles, iron oxides, and/or ferritins are tested for its in vitro and/or in vivo expression of IL-12 and/or IL-2, as well as its efficacy as a cancer therapeutic. These results are not available at the time of filing.

### Example 48: A Recombinant Nucleic Acid as Described in any One or More of Examples 7-12 in One or More Nanocarriers

A recombinant nucleic acid is engineered as described in any one or more of examples 7-12 in one or more nanocarriers as a non-viral gene delivery vehicle. The one or more nanocarriers are tested for its in vitro and/or in vivo expression of IL-12 and/or IL-2, as well as its efficacy as a cancer therapeutic. These results are not available at the time of filing.

### Example 49: A Recombinant Nucleic Acid as Described in any One or More of Examples 7-12 in One or More DNA Nanclews

A recombinant nucleic acid is engineered as described in any one or more of examples 7-12 in one or more DNA nanclews as a non-viral gene delivery vehicle. The one or more DNA nanclews are tested for its in vitro and/or in vivo expression of IL-12 and/or IL-2, as well as its efficacy as a cancer therapeutic. These results are not available at the time of filing.

### Example 50: A Recombinant Nucleic Acid as Described in any One or More of Examples 7-12 in One or More Gold Nanoparticles

A recombinant nucleic acid is engineered as described in any one or more of examples 7-12 in one or more gold nanoparticles as a non-viral gene delivery vehicle. The one or more gold nanoparticles are tested for its in vitro and/or in vivo expression of IL-12 and/or IL-2, as well as its efficacy as a cancer therapeutic. These results are not available at the time of filing.

### Example 51: A Recombinant Nucleic Acid as Described in any One or More of Examples 7-12 in One or More Carbon Nanotubes

A recombinant nucleic acid is engineered as described in any one or more of examples 7-12 in one or more carbon nanotubes as a non-viral gene delivery vehicle. The one or more carbon nanotubes are tested for its in vitro and/or in vivo expression of IL-12 and/or IL-2, as well as its efficacy as a cancer therapeutic. These results are not available at the time of filing.

### Example 52: A Recombinant Nucleic Acid as Described in any One or More of Examples 7-12 in One or More Graphenes

A recombinant nucleic acid is engineered as described in any one or more of examples 7-12 in one or more graphenes as a non-viral gene delivery vehicle. The one or more graphenes are tested for its in vitro and/or in vivo expression of IL-12 and/or IL-2, as well as its efficacy as a cancer therapeutic. These results are not available at the time of filing.

### Example 53: A Recombinant Nucleic Acid as Described in any One or More of Examples 7-12 in One or More Quantum Dots

A recombinant nucleic acid is engineered as described in any one or more of examples 7-12 in one or more quantum dots as a non-viral gene delivery vehicle. The one or more quantum dots are tested for its in vitro and/or in vivo expression of IL-12 and/or IL-2, as well as its efficacy as a cancer therapeutic. These results are not available at the time of filing.

### Example 54: A Recombinant Nucleic Acid as Described in any One or More of Examples 7-12 in One or More Up-Conversion Nanoparticles

A recombinant nucleic acid is engineered as described in any one or more of examples 7-12 in one or more up-conversion nanoparticles as a non-viral gene delivery vehicle. The one or more up-conversion nanoparticles are tested for its in vitro and/or in vivo expression of IL-12 and/or IL-2, as well as its efficacy as a cancer therapeutic. These results are not available at the time of filing.

### Example 55: A Recombinant Nucleic Acid as Described in any One or More of Examples 7-12 in One or More Silica Nanoparticles

A recombinant nucleic acid is engineered as described in any one or more of examples 7-12 in one or more silica nanoparticles as a non-viral gene delivery vehicle. The one or more silica nanoparticles are tested for its in vitro and/or in vivo expression of IL-12 and/or IL-2, as well as its efficacy as a cancer therapeutic. These results are not available at the time of filing.

### Example 56: A Recombinant Nucleic Acid as Described in any One or More of Examples 7-12 in One or More Iron Oxides

A recombinant nucleic acid is engineered as described in any one or more of examples 7-12 in one or more iron oxides as a non-viral gene delivery vehicle. The one or more iron oxides are tested for its in vitro and/or in vivo expression of IL-12 and/or IL-2, as well as its efficacy as a cancer therapeutic. These results are not available at the time of filing.

### Example 57: A Recombinant Nucleic Acid as Described in any One or More of Examples 7-12 in One or More Ferritins

A recombinant nucleic acid is engineered as described in any one or more of examples 7-12 in one or more ferritins as a non-viral gene delivery vehicle. The one or more ferritins are tested for its in vitro and/or in vivo expression of IL-12 and/or IL-2, as well as its efficacy as a cancer therapeutic. These results are not available at the time of filing.

### Example 58: A Recombinant Nucleic Acid as Described in any One or More of Examples 7-12 Delivered by One or More of Electroporation, Gene Gun, Jet Gun, Ultrasound, Nucleofection, Hydrodynamic Gene Delivery, Needle Injection, Microinjection, Ballistic DNA Injection, Sonoporation, Photoporation, Laser Pulse, Magnetofection, Magnetoporation, Magnetic Particles, and/or Hydroporation

A recombinant nucleic acid is engineered as described in any one or more of examples 7-12 delivered by one or more of electroporation, gene gun, jet gun, ultrasound, nucleofection, hydrodynamic gene delivery, needle injection, microinjection, ballistic DNA injection, sonoporation, photoporation, laser pulse, magnetofection, magnetoporation, magnetic particles, and/or hydroporation as a non-viral gene delivery vehicle. The delivery by one or more of electroporation, gene gun, jet gun, ultrasound, nucleofection, hydrodynamic gene delivery, needle injection, microinjection, ballistic DNA injection, sonoporation, photoporation, laser pulse, magnetofection, magnetoporation, magnetic particles, and/or hydroporation is tested for its in vitro and/or in vivo expression of IL-12 and/or IL-2, as well as its efficacy as a cancer therapeutic. These results are not available at the time of filing.

### Example 59: A Recombinant Nucleic Acid as Described in any One or More of Examples 7-12 Delivered by Electroporation

A recombinant nucleic acid is engineered as described in any one or more of examples 7-12 delivered by electroporation as a non-viral gene delivery vehicle. The delivery by electroporation is tested for its in vitro and/or in vivo expression of IL-12 and/or IL-2, as well as its efficacy as a cancer therapeutic. These results are not available at the time of filing.

### Example 60: A Recombinant Nucleic Acid as Described in any One or More of Examples 7-12 Delivered by Gene Gun

A recombinant nucleic acid is engineered as described in any one or more of examples 7-12 delivered by gene gun as a non-viral gene delivery vehicle. The delivery by gene gun is tested for its in vitro and/or in vivo expression of IL-12 and/or IL-2, as well as its efficacy as a cancer therapeutic. These results are not available at the time of filing.

### Example 61: A Recombinant Nucleic Acid as Described in any One or More of Examples 7-12 Delivered by Jet Gun

A recombinant nucleic acid is engineered as described in any one or more of examples 7-12 delivered by jet gun as a non-viral gene delivery vehicle. The delivery by jet gun is tested for its in vitro and/or in vivo expression of IL-12 and/or IL-2, as well as its efficacy as a cancer therapeutic. These results are not available at the time of filing.

### Example 62: A Recombinant Nucleic Acid as Described in any One or More of Examples 7-12 Delivered by Ultrasound

A recombinant nucleic acid is engineered as described in any one or more of examples 7-12 delivered by ultrasound as a non-viral gene delivery vehicle. The delivery by ultrasound is tested for its in vitro and/or in vivo expression of IL-12 and/or IL-2, as well as its efficacy as a cancer therapeutic. These results are not available at the time of filing.

### Example 63: A Recombinant Nucleic Acid as Described in any One or More of Examples 7-12 Delivered by Nucleofection

A recombinant nucleic acid is engineered as described in any one or more of examples 7-12 delivered by nucleofection as a non-viral gene delivery vehicle. The delivery by nucleofection is tested for its in vitro and/or in vivo expression of IL-12 and/or IL-2, as well as its efficacy as a cancer therapeutic. These results are not available at the time of filing.

### Example 64: A Recombinant Nucleic Acid as Described in any One or More of Examples 7-12 Delivered by Hydrodynamic Gene Delivery

A recombinant nucleic acid is engineered as described in any one or more of examples 7-12 delivered by hydrodynamic gene delivery as a non-viral gene delivery vehicle. The delivery by hydrodynamic gene delivery is tested for its in vitro and/or in vivo expression of IL-12 and/or IL-2, as well as its efficacy as a cancer therapeutic. These results are not available at the time of filing.

### Example 65: A Recombinant Nucleic Acid as Described in any One or More of Examples 7-12 Delivered by Needle Injection

A recombinant nucleic acid is engineered as described in any one or more of examples 7-12 delivered by needle injection as a non-viral gene delivery vehicle. The delivery by needle injection is tested for its in vitro and/or in vivo expression of IL-12 and/or IL-2, as well as its efficacy as a cancer therapeutic. These results are not available at the time of filing.

### Example 66: A Recombinant Nucleic Acid as Described in any One or More of Examples 7-12 Delivered by Microinjection

A recombinant nucleic acid is engineered as described in any one or more of examples 7-12 delivered by microinjection as a non-viral gene delivery vehicle. The delivery by microinjection is tested for its in vitro and/or in vivo expression of IL-12 and/or IL-2, as well as its efficacy as a cancer therapeutic. These results are not available at the time of filing.

### Example 67: A Recombinant Nucleic Acid as Described in any One or More of Examples 7-12 Delivered by Ballistic DNA Injection

A recombinant nucleic acid is engineered as described in any one or more of examples 7-12 delivered by ballistic DNA injection as a non-viral gene delivery vehicle. The delivery by ballistic DNA injection is tested for its in vitro and/or in vivo expression of IL-12 and/or IL-2, as well as its efficacy as a cancer therapeutic. These results are not available at the time of filing.

### Example 68: A Recombinant Nucleic Acid as Described in any One or More of Examples 7-12 Delivered by Sonoporation

A recombinant nucleic acid is engineered as described in any one or more of examples 7-12 delivered by sonoporation as a non-viral gene delivery vehicle. The delivery by sonoporation is tested for its in vitro and/or in vivo expression of IL-12 and/or IL-2, as well as its efficacy as a cancer therapeutic. These results are not available at the time of filing.

### Example 69: A Recombinant Nucleic Acid as Described in any One or More of Examples 7-12 Delivered by Photoporation

A recombinant nucleic acid is engineered as described in any one or more of examples 7-12 delivered by photoporation as a non-viral gene delivery vehicle. The delivery by photoporation is tested for its in vitro and/or in vivo expression of IL-12 and/or IL-2, as well as its efficacy as a cancer therapeutic. These results are not available at the time of filing.

### Example 70: A Recombinant Nucleic Acid as Described in any One or More of Examples 7-12 Delivered by Laser Pulse

A recombinant nucleic acid is engineered as described in any one or more of examples 7-12 delivered by laser pulse as a non-viral gene delivery vehicle. The delivery by laser pulse is tested for its in vitro and/or in vivo expression of IL-12 and/or IL-2, as well as its efficacy as a cancer therapeutic. These results are not available at the time of filing.

### Example 71: A Recombinant Nucleic Acid as Described in any One or More of Examples 7-12 Delivered by Magnetofection

A recombinant nucleic acid is engineered as described in any one or more of examples 7-12 delivered by magnetofection as a non-viral gene delivery vehicle. The delivery by magnetofection is tested for its in vitro and/or in vivo expression of IL-12 and/or IL-2, as well as its efficacy as a cancer therapeutic. These results are not available at the time of filing.

### Example 72: A Recombinant Nucleic Acid as Described in any One or More of Examples 7-12 Delivered by Magnetoporation

A recombinant nucleic acid is engineered as described in any one or more of examples 7-12 delivered by magnetoporation as a non-viral gene delivery vehicle. The delivery by magnetoporation is tested for its in vitro and/or in vivo expression of IL-12 and/or IL-2, as well as its efficacy as a cancer therapeutic. These results are not available at the time of filing.

### Example 73: A Recombinant Nucleic Acid as Described in any One or More of Examples 7-12 Delivered by Magnetic Particles

A recombinant nucleic acid is engineered as described in any one or more of examples 7-12 delivered by magnetic particles as a non-viral gene delivery vehicle. The delivery by magnetic particles is tested for its in vitro and/or in vivo expression of IL-12 and/or IL-2, as well as its efficacy as a cancer therapeutic. These results are not available at the time of filing.

### Example 74: A Recombinant Nucleic Acid as Described in any One or More of Examples 7-12 Delivered by Hydroporation

A recombinant nucleic acid is engineered as described in any one or more of examples 7-12 delivered by hydroporation as a non-viral gene delivery vehicle. The delivery by hydroporation is tested for its in vitro and/or in vivo expression of IL-12 and/or IL-2, as well as its efficacy as a cancer therapeutic. These results are not available at the time of filing.

What is claimed is:

1. A composition comprising a first recombinant polynucleotide encoding an Interleukin (IL)-2 polypeptide and a second recombinant polynucleotide encoding an IL-12 polypeptide.

2. A pharmaceutical composition comprising the first recombinant polynucleotide and the second recombinant polynucleotide of claim 1.

3. The pharmaceutical composition of claim 2, wherein the pharmaceutical composition comprises a recombinant nucleic acid comprising the first polynucleotide and a recombinant nucleic acid comprising the second polynucleotide.

4. The pharmaceutical composition of claim 3, wherein the recombinant nucleic acid comprising the first polynucleotide and the recombinant nucleic acid comprising the second polynucleotide are the same recombinant nucleic acid.

5. The pharmaceutical composition of claim 3, wherein the recombinant nucleic acid comprising the first polynucleotide and the recombinant nucleic acid comprising the second polynucleotide are different recombinant nucleic acids.

6. The pharmaceutical composition of any one of claims 2-5, wherein the pharmaceutical composition comprises one or more gene delivery vehicles comprising the first polynucleotide and the second polynucleotide.

7. The pharmaceutical composition of any one of claims 2-6, wherein the first polynucleotide and the second polynucleotide are contiguous.

8. The pharmaceutical composition of any one of claims 2-6, wherein the first polynucleotide and the second polynucleotide are non-contiguous.

9. The pharmaceutical composition of any one of claims 2-8, wherein the first polynucleotide and/or the second polynucleotide comprises a deoxyribonucleic acid (DNA).

10. The pharmaceutical composition of any one of claims 2-9, wherein the DNA is a linear DNA, a circular DNA, or a close ended DNA (ceDNA).

11. The pharmaceutical composition of any one of claims 2-8, wherein the first polynucleotide and/or the second polynucleotide comprises a ribonucleic acid (RNA), a messenger RNA (mRNA), or a modified mRNA (mmRNA).

12. The pharmaceutical composition of claim 11, wherein the mRNA and/or the mmRNA further comprises a 5′ untranslated region (UTR), a 3′ UTR, a polyadenylation (poly(A)) tail, and/or a 5′ cap analog.

13. The pharmaceutical composition of claim 12, wherein the 5′ cap analog is 7mG(5′)ppp(5′)NImpNp.

14. The pharmaceutical composition of any one of claims 11-13, wherein the mRNA and/or the mmRNA further comprises a 1-methylpseudouridine modification and/or a 1-ethylpseudouridine modification.

15. The pharmaceutical composition of any one of claims 2-14, wherein the first polynucleotide and/or the second polynucleotide are codon-optimized.

16. The pharmaceutical composition of any one of claims 2-15, wherein the first polynucleotide and/or the second polynucleotide are codon-optimized for human codon usage.

17. The pharmaceutical composition of any one of claims 2-16, wherein the IL-2 polypeptide is a human IL-2 polypeptide.

18. The pharmaceutical composition of any one of claims 2-17, wherein the IL-2 polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 1.

19. The pharmaceutical composition of any one of claims 2-18, wherein the IL-12 polypeptide is a human IL-12 polypeptide.

20. The pharmaceutical composition of any one of claims 2-19, wherein the IL-12 polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NOs: 2-4.

21. The pharmaceutical composition of any one of claims 6-20, wherein the one or more gene delivery vehicles are one or more viral gene delivery vehicles and/or one or more non-viral gene delivery vehicles.

22. The pharmaceutical composition of claim 21, wherein the one or more viral gene delivery vehicles comprise a recombinant viral genome.

23. The pharmaceutical composition of claim 22, wherein the recombinant viral genome comprises the first polynucleotide and/or the second polynucleotide.

24. The pharmaceutical composition of claim 22 or claim 23, wherein the recombinant viral genome is replication competent or replication defective.

25. The pharmaceutical composition of any one of claims 22-24, wherein the recombinant viral genome is a recombinant oncolytic virus genome, or wherein the recombinant viral genome is not a recombinant oncolytic virus genome.

26. The pharmaceutical composition of any one of claims 22-25, wherein the recombinant viral genome is selected from the group consisting of a recombinant adenovirus genome, a recombinant retrovirus genome, a recombinant adeno-associated virus genome, a recombinant herpes virus genome, a recombinant poxvirus genome, a recombinant bacteriophage genome, a recombinant alphavirus genome, a recombinant picornavirus genome, a recombinant iridovirus genome, a recombinant Newcastle disease virus genome, a recombinant baculovirus genome, a recombinant geminivirus genome, a recombinant caulimovirus genome, a recombinant anellovirus genome, and a combination thereof.

27. The pharmaceutical composition of claim 26, wherein the recombinant adenovirus genome is selected from the group consisting of a recombinant Atadenovirus genome, a recombinant Aviadenovirus genome, a recombinant Ichtadenovirus genome, a recombinant Mastadenovirus genome, a recombinant Siadenovirus genome, a recombinant Testadenovirus genome, and any derivatives thereof.

28. The pharmaceutical composition of claim 26, wherein the recombinant retrovirus genome is selected from the group consisting of a recombinant Alpharetrovirus genome, a recombinant Betaretrovirus genome, a recombinant Gammaretrovirus genome, a recombinant Deltaretrovirus genome, a recombinant Epsilonretrovirus genome, a recombinant Lentivirus genome, a recombinant Bovispumavirus genome, a recombinant Equispumavirus genome, a recombinant Felispumavirus genome, a recombinant Prosimiispumavirus genome, a recombinant Simiispumavirus genome, and any derivatives thereof.

29. The pharmaceutical composition of claim 26, wherein the recombinant adeno-associated virus genome is selected from the group consisting of a recombinant Adeno-associated dependoparvovirus A genome, a recombinant Adeno-associated dependoparvovirus B genome, and any derivates thereof.

30. The pharmaceutical composition of claim 29, wherein the recombinant adeno-associated virus (AAV) genome is a recombinant AAV serotype 1 genome, a recombinant AAV serotype 2 genome, a recombinant AAV serotype 3 genome, a recombinant AAV serotype 4 genome, a recombinant AAV serotype 5 genome, a recombinant AAV serotype 6 genome, a recombinant AAV serotype 7 genome, a recombinant AAV serotype 8 genome, a recombinant AAV serotype 9 genome, a recombinant AAV serotype 10 genome. a recombinant AAV serotype 11 genome, or a combination thereof.

31. The pharmaceutical composition of claim 26, wherein the recombinant herpes virus genome is selected from the group consisting of a recombinant herpes simplex virus genome, a recombinant varicella zoster virus genome, a recombinant human cytomegalovirus genome, a recombinant herpesvirus 6A genome, a recombinant herpesvirus 6B genome, a recombinant herpesvirus 7 genome, a recombinant Epstein-Barr virus genome, a recombinant Kaposi's sarcoma-associated herpesvirus genome, and any derivatives thereof.

32. The pharmaceutical composition of claim 31, wherein the recombinant herpes simplex virus genome is a recombinant herpes simplex virus type 1 (HSV-1) genome, a recombinant herpes simplex virus type 2 (HSV-2) genome, or any derivatives thereof.

33. The pharmaceutical composition of claim 26, wherein the recombinant poxvirus genome is selected from the group consisting of a recombinant smallpox virus genome, a recombinant vaccinia virus genome, a recombinant cowpox virus genome, a recombinant monkeypox virus genome, a recombinant orf virus genome, a recombinant pseudocowpox virus genome, a recombinant bovine papular stomatitis virus genome, a recombinant tanapox virus genome, a recombinant yaba monkey tumor virus genome, a recombinant molluscum contagiosum virus genome, and any derivatives thereof.

34. The pharmaceutical composition of claim 26, wherein the recombinant bacteriophage genome is selected from the group consisting of a recombinant 186 phage genome, a recombinant Escherichia virus Lambda genome, a recombinant Pseudomonas virus phi6 genome, a recombinant Bacillus virus φ29 genome, a recombinant Escherichia virus φX174 genome, a recombinant Bacteriophage φCb5 genome, a recombinant Escherichia virus G4 genome, a recombinant Escherichia virus M13 genome, a recombinant Emesvirus zinderi genome, a recombinant N4 phage genome, a recombinant Escherichia virus P1 genome, a recombinant Escherichia virus P2 genome, a recombinant Enterobacteria phage P4 genome, a recombinant R17 phage genome, a recombinant Enterobacteria phage T2 genome, a recombinant Escherichia virus T4 genome, a recombinant Escherichia virus T7 genome, a recombinant Bacteriophage T12 genome, and any derivatives thereof.

35. The pharmaceutical composition of claim 26, wherein the recombinant alphavirus genome is selected from the group consisting of a recombinant Aura virus genome, a recombinant Barmah Forest virus genome, a recombinant Bebaru virus genome, a recombinant Caaingua virus genome, a recombinant Cabassou virus genome, a recombinant Chikungunya virus genome, a recombinant Eastern equine encephalitis virus genome, a recombinant Eilat virus genome, a recombinant Everglades virus genome, a recombinant Fort Morgan virus genome, a recombinant Getah virus genome, a recombinant Highlands J virus genome, a recombinant Madariaga virus genome, a recombinant Mayaro virus genome, a recombinant Middelburg virus genome, a recombinant Mosso das Pedras virus genome, a recombinant Mucambo virus genome, a recombinant Ndumu virus genome, a recombinant O'nyong'nyong virus genome, a recombinant Pixuna virus genome, a recombinant Rio Negro virus genome, a recombinant Ross River virus genome, a recombinant Salmon pancreas disease virus genome, a recombinant Semliki Forest virus genome, a recombinant Sindbis virus genome, a recombinant Southern elephant seal virus genome, a recombinant Tonate virus genome, a recombinant Trocara virus genome, a recombinant Una virus genome, a recombinant Venezuelan equine encephalitis virus genome, a recombinant Western equine encephalitis virus genome, a recombinant Whataroa virus genome, and any derivatives thereof.

36. The pharmaceutical composition of claim 26, wherein the recombinant picornavirus genome is selected from the group consisting of a recombinant Aalivirus genome, a recombinant Ailurivirus genome, a recombinant Ampivirus genome, a recombinant Antivirus genome, a recombinant Aphthovirus genome, a recombinant Aquamavirus genome, a recombinant Avihepatovirus genome, a recombinant Avisivirus genome, a recombinant Boosepivirus genome, a recombinant Bopivirus genome, a recombinant Caecilivirus genome, a recombinant Cardiovirus genome, a recombinant Cosavirus genome, a recombinant Crahelivirus genome, a recombinant Crohivirus genome, a recombinant Danipivirus genome, a recombinant Dicipivirus genome, a recombinant Diresapivirus genome, a recombinant Enterovirus genome, a recombinant Erbovirus genome, a recombinant Felipivirus genome, a recombinant Fipivirus genome, a recombinant Gallivirus genome, a recombinant Gruhelivirus genome, a recombinant Grusopivirus genome, a recombinant Harkavirus genome, a recombinant Hemipivirus genome, a recombinant Hepatovirus genome, a recombinant Hunnivirus genome, a recombinant Kobuvirus genome, a recombinant Kunsagivirus genome, a recombinant Limnipivirus genome, a recombinant Livupivirus genome, a recombinant Ludopivirus genome, a recombinant Malagasivirus genome, a recombinant Marsupivirus genome, a recombinant Megrivirus genome, a recombinant Mischivirus genome, a recombinant Mosavirus genome, a recombinant Mupivirus genome, a recombinant Myrropivirus genome, a recombinant Orivirus genome, a recombinant Oscivirus genome, a recombinant Parabovirus genome, a recombinant Parechovirus genome, a recombinant Pasivirus genome, a recombinant Passerivirus genome, a recombinant Pemapivirus genome, a recombinant Poecivirus genome, a recombinant Potamipivirus genome, a recombinant Pygoscepivirus genome, a recombinant Rabovirus genome, a recombinant Rafivirus genome, a recombinant Rajidapivirus genome, a recombinant Rohelivirus genome, a recombinant Rosavirus genome, a recombinant Sakobuvirus genome, a recombinant Salivirus genome, a recombinant Sapelovirus genome, a recombinant Senecavirus genome, a recombinant Shanbavirus genome, a recombinant Sicinivirus genome, a recombinant Symapivirus genome, a recombinant Teschovirus genome, a recombinant Torchivirus genome, a recombinant Tottorivirus genome, a recombinant Tremovirus genome, a recombinant Tropivirus genome, and any derivatives thereof.

37. The pharmaceutical composition of claim 26, wherein the recombinant iridovirus genome is a recombinant Invertebrate iridescent virus 6 (IIV-6) genome or a recombinant Invertebrate iridescent virus 31 (IIV-31) genome.

38. The pharmaceutical composition of claim 26, wherein the recombinant baculovirus genome is selected from the group consisting of a recombinant alphabaculovirus genome, a recombinant betabaculovirus genome, a recombinant deltabaculovirus genome, a recombinant gammabaculovirus genome, and any derivatives thereof.

39. The pharmaceutical composition of claim 26, wherein the recombinant geminivirus genome is selected from the group consisting of a recombinant Becurtovirus genome, a recombinant Begomovirus genome, a recombinant Capulavirus genome, a recombinant Citlodavirus genome, a recombinant Curtovirus genome, a recombinant Eragrovirus genome, a recombinant Grablovirus genome, a recombinant Maldovirus genome, a recombinant Mastrevirus genome, a recombinant Mulcrilevirus genome, a recombinant Opunvirus genome, a recombinant Topilevirus genome, a recombinant Topocuvirus genome, a recombinant Turncurtovirus genome, and any derivatives thereof.

40. The pharmaceutical composition of claim 26, wherein the recombinant caulimovirus genome is selected from the group consisting of a recombinant Angelica bushy stunt virus genome, a recombinant Atractylodes mild mottle virus genome, a recombinant Carnation etched ring virus genome, a recombinant Cauliflower mosaic virus genome, a recombinant Dahlia mosaic virus genome, a recombinant Figwort mosaic virus genome, a recombinant Horseradish latent virus genome, a recombinant Lamium leaf distortion virus genome, a recombinant Mirabilis mosaic virus genome, a recombinant Soybean Putnam virus genome, a recombinant Strawberry vein banding virus genome, a recombinant Thistle mottle virus genome, and any derivatives thereof.

41. The pharmaceutical composition of any one of claims 6-21, wherein the gene delivery vehicle is the one or more non-viral gene delivery vehicles.

42. The pharmaceutical composition of claim 41, wherein the one or more non-viral gene delivery vehicles is a chemical compound, a bacterium, a mammalian cell, or a physical delivery system.

43. The pharmaceutical composition of claim 42, wherein the chemical compound is selected from the groups consisting of a polymer compound, a lipid compound, an inorganic compound, and a combination thereof.

44. The pharmaceutical composition of claim 43, wherein the polymer compound is a natural polymer, a synthetic polymer, a biopolymer, a biodegradable polymer, a cationic polymer, a protein polymer, a polysaccharide polymer, or any combination thereof.

45. The pharmaceutical composition of claim 43 or claim 44, wherein the polymer compound is selected from the group consisting of poly-L-lactide (PLA), chitosan, pectin, polyethylenimine (PEI), poly-L-lysine (PLL), poly 2-N-dimethylaminoethyl methacrylate (PDMAEMA), polyaminoamine, polyaminoesters, polyamidoamine, poly-vinylimidazole (PVI), Poly-beta-amino ester (PBAE), gelatin, albumin, chitosan, dextran, hyaluronic beta-cyclodextrin, acid, polyester, alpha-Pyrrolidinohexiophenone (PHP), Poly-2-acrylamidoglycolic acid (PAGA), Poly-delta-valerolactone (PVL), polyphenyl ethers (PPE), Poly-beta-amino-ester (PBAE), poly-amine-co-ester (PACE), poly-lactic-co-glycolic acid (PLGA), polycarbonates, polyurethanes, micelles, cationic emulsion, exosome, Reb Blood Cell membrane, DNA-protein complexes, plasmid DNA, naked DNA, pBR322, pUC, and any derivatives thereof.

46. The pharmaceutical composition of claim 43, wherein the lipid compound is selected from the group consisting of a nanoparticle, a lipid nanoparticle, a liposome, a cationic liposome, a solid lipid nanoparticle, a lipid emulsion, a lipidoid, a cytofectin, a surfactant, a gemini surfactant, and any derivatives thereof.

47. The pharmaceutical composition of claim 43, wherein the inorganic compound is selected from the group consisting of a nanocarrier, a DNA nanclew, a gold nanoparticle, a carbon nanotube, a graphene, a quantum dot, an up-conversion nanoparticle, a silica nanoparticle, an iron oxide, a ferritin, and any derivatives thereof.

48. The pharmaceutical composition of claim 42, wherein the physical delivery system is selected from the group consisting of electroporation, gene gun, jet gun, ultrasound, nucleofection, hydrodynamic gene delivery, needle injection, microinjection, ballistic DNA injection, sonoporation, photoporation, laser pulse, magnetofection, magnetoporation, magnetic particles, hydroporation, and any combination thereof.

49. Use of the pharmaceutical composition of any one of claims 2-48 in the manufacture of a medicament for treating cancer.

50. The use of claim 49, wherein the cancer is selected from the group consisting of acute myeloid leukemia, acute lymphoblastic leukemia, adrenocortical carcinoma, bladder urothelial cancer, brain stem glioma, brain lower grade glioma, brain tumor, breast cancer, bronchial tumors, Burkitt lymphoma, cancer of unknown primary site, carcinoid tumor, carcinoma of unknown primary site, central nervous system atypical teratoid/rhabdoid tumor, central nervous system embryonal tumors, cervical squamous cell carcinoma, endocervical adenocarcinoma cancer, childhood cancers, cholangiocarcinoma, chordoma, chronic lymphocytic leukemia, chronic myelogenous leukemia, chronic myeloproliferative disorders, colon cancer, colorectal cancer, craniopharyngioma, cutaneous T-cell lymphoma, endocrine pancreas islet cell tumors, endometrial cancer, ependymoblastoma, ependymoma, esophageal cancer, esthesioneuroblastoma, Ewing sarcoma, extracranial germ cell tumor, extragonadal germ cell tumor, extrahepatic bile duct cancer, gallbladder cancer, gastric cancer, gastrointestinal carcinoid tumor, gastrointestinal stromal cell tumor, gastrointestinal stromal tumor, gestational trophoblastic tumor, glioblastoma multiforme glioma, hairy cell leukemia, head and neck cancer, heart cancer, Hodgkin lymphoma, hypopharyngeal cancer, intraocular melanoma, islet cell tumors, Kaposi sarcoma, kidney cancer, Langerhans cell histiocytosis, laryngeal cancer, leptomeningeal cancer, lip cancer, liver cancer, Lymphoid Neoplasm Diffuse Large B-cell Lymphoma, malignant fibrous histiocytoma bone cancer, medulloblastoma, medulloepithelioma, melanoma, Merkel cell carcinoma, Merkel cell skin carcinoma, mesothelioma, metastatic squamous neck cancer with occult primary, mouth cancer, multiple endocrine neoplasia syndromes, multiple myeloma, multiple myeloma/plasma cell neoplasm, mycosis fungoides, myelodysplastic syndromes, myeloproliferative neoplasms, nasal cavity cancer, nasopharyngeal cancer, neuroblastoma, Non-Hodgkin lymphoma, nonmelanoma skin cancer, non-small cell lung cancer, oral cancer, oral cavity cancer, oropharyngeal cancer, osteosarcoma, other brain and spinal cord tumors, ovarian cancer, ovarian epithelial cancer, ovarian germ cell tumor, ovarian low malignant potential tumor, pancreatic cancer, papillomatosis, paranasal sinus cancer, parathyroid cancer, pelvic cancer, penile cancer, pharyngeal cancer, pheochromocytoma and paraganglioma, pineal parenchymal tumors of intermediate differentiation, pineoblastoma, pituitary tumor, plasma cell neoplasm/multiple myeloma, pleuropulmonary blastoma, primary central nervous system lymphoma, primary hepatocellular liver cancer, prostate cancer such as prostate adenocarcinoma, rectal cancer, renal cancer, renal cell cancer, renal cell cancer, respiratory tract cancer, retinoblastoma, rhabdomyosarcoma, salivary gland cancer, sarcoma, Sezary syndrome, skin cutaneous melanoma, small cell lung cancer, small intestine cancer, soft tissue sarcoma, squamous cell carcinoma, squamous neck cancer, stomach cancer, supratentorial primitive neuroectodermal tumors, T-cell lymphoma, testicular cancer testicular germ cell tumors, throat cancer, thymic carcinoma, thymoma, thyroid cancer, transitional cell cancer, transitional cell cancer of the renal pelvis and ureter, trophoblastic tumor, ureter cancer, urethral cancer, uterine cancer, uterine cancer, uveal melanoma, vaginal cancer, vulvar cancer, Waldenstrom macroglobulinemia, and Wilms' tumor.

51. A method of expressing, enhancing, increasing, augmenting, and/or supplementing the levels of an IL-2 polypeptide and/or an IL-12 polypeptide in one or more cells of a subject, the method comprising administering to the subject an effective amount of the pharmaceutical composition of any one of claims 2-48.

52. The method of claim 51, wherein the one or more cells are one or more cells of the respiratory tract, airway epithelia, and/or lung.

53. A method of providing prophylactic, palliative, or therapeutic relief of one or more signs or symptoms of cancer in a subject in need thereof, the method comprising administering to the subject an effective amount of the pharmaceutical composition of any one of claims 2-48.

54. A method of treating cancer in a subject in need thereof, the method comprising administering to the subject an effective amount of the pharmaceutical composition of any one of claims 2-48.

55. The method of claim 53 or claim 54, wherein the cancer is selected from the group consisting of acute myeloid leukemia, acute lymphoblastic leukemia, adrenocortical carcinoma, bladder urothelial cancer, brain stem glioma, brain lower grade glioma, brain tumor, breast cancer, bronchial tumors, Burkitt lymphoma, cancer of unknown primary site, carcinoid tumor, carcinoma of unknown primary site, central nervous system atypical teratoid/rhabdoid tumor, central nervous system embryonal tumors, cervical squamous cell carcinoma, endocervical adenocarcinoma cancer, childhood cancers, cholangiocarcinoma, chordoma, chronic lymphocytic leukemia, chronic myelogenous leukemia, chronic myeloproliferative disorders, colon cancer, colorectal cancer, craniopharyngioma, cutaneous T-cell lymphoma, endocrine pancreas islet cell tumors, endometrial cancer, ependymoblastoma, ependymoma, esophageal cancer, esthesioneuroblastoma, Ewing sarcoma, extracranial germ cell tumor, extragonadal germ cell tumor, extrahepatic bile duct cancer, gallbladder cancer, gastric cancer, gastrointestinal carcinoid tumor, gastrointestinal stromal cell tumor, gastrointestinal stromal tumor, gestational trophoblastic tumor, glioblastoma multiforme glioma, hairy cell leukemia, head and neck cancer, heart cancer, Hodgkin lymphoma, hypopharyngeal cancer, intraocular melanoma, islet cell tumors, Kaposi sarcoma, kidney cancer, Langerhans cell histiocytosis, laryngeal cancer, leptomeningeal cancer, lip cancer, liver cancer, Lymphoid Neoplasm Diffuse Large B-cell Lymphoma, malignant fibrous histiocytoma bone cancer, medulloblastoma, medulloepithelioma, melanoma, Merkel cell carcinoma, Merkel cell skin carcinoma, mesothelioma, metastatic squamous neck cancer with occult primary, mouth cancer, multiple endocrine neoplasia syndromes, multiple myeloma, multiple myeloma/plasma cell neoplasm, mycosis fungoides, myelodysplastic syndromes, myeloproliferative neoplasms, nasal cavity cancer, nasopharyngeal cancer, neuroblastoma, Non-Hodgkin lymphoma, nonmelanoma skin cancer, non-small cell lung cancer, oral cancer, oral cavity cancer, oropharyngeal cancer, osteosarcoma, other brain and spinal cord tumors, ovarian cancer, ovarian epithelial cancer, ovarian germ cell tumor, ovarian low malignant potential tumor, pancreatic cancer, papillomatosis, paranasal sinus cancer, parathyroid cancer, pelvic cancer, penile cancer, pharyngeal cancer, pheochromocytoma and paraganglioma, pineal parenchymal tumors of intermediate differentiation, pineoblastoma, pituitary tumor, plasma cell neoplasm/multiple myeloma, pleuropulmonary blastoma, primary central nervous system lymphoma, primary hepatocellular liver cancer, prostate cancer such as prostate adenocarcinoma, rectal cancer, renal cancer, renal cell cancer, renal cell cancer, respiratory tract cancer, retinoblastoma, rhabdomyosarcoma, salivary gland cancer, sarcoma, Sezary syndrome, skin cutaneous melanoma, small cell lung cancer, small intestine cancer, soft tissue sarcoma, squamous cell carcinoma, squamous neck cancer, stomach cancer, supratentorial primitive neuroectodermal tumors, T-cell lymphoma, testicular cancer testicular germ cell tumors, throat cancer, thymic carcinoma, thymoma, thyroid cancer, transitional cell cancer, transitional cell cancer of the renal pelvis and ureter, trophoblastic tumor, ureter cancer, urethral cancer, uterine cancer, uterine cancer, uveal melanoma, vaginal cancer, vulvar cancer, Waldenstrom macroglobulinemia, and Wilms' tumor.

56. The method of any one of claims 53-55, wherein the cancer is selected from the group consisting of a solid tumor, a hematologic cancer, bladder cancer, brain cancer, breast cancer, colon cancer, gastric cancer, glioma, head cancer, leukemia, liver cancer, lung cancer, lymphoma, myeloma, neck cancer, ovarian cancer, melanoma, pancreatic cancer, renal cancer, salivary cancer, stomach cancer, thymic epithelial cancer, and thyroid cancer.

57. The method of any one of claims 53-56, wherein the cancer is small-cell lung cancer, non-small cell lung cancer, adenocarcinoma of the lung, or squamous carcinoma of the lung.

58. The method of any one of claims 53-57, wherein the cancer is non-small cell lung cancer.

59. The method of any one of claims 51-58, wherein the subject is a human.

60. The method of any one of claims 51-59, wherein the pharmaceutical composition is administered topically, transdermally, subcutaneously, epicutaneously, intradermally, orally, sublingually, buccally, rectally, vaginally, intravenously, intraarterially, intramuscularly, intraosseously, intracardially, intraperitoneally, transmucosally, intravitreally, subretinally, suprachoroidally, intracranially, intrathecally, intraventricularly, intraarticularly, peri-articularly, intratumorally, locally, or via inhalation to the subject.

61. The method of any one of claims 51-60, wherein the pharmaceutical composition is administered orally, intranasally, intratracheally, or via inhalation to the subject.

62. The method of any one of claims 51-60, wherein the pharmaceutical composition is administered intratumorally to the subject.


SEQUENCE LISTING (31 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 3 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 4 - protein, 545 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 5 - DNA, 462 bp, Homo sapiens
  source 1..462: mol_type = genomic DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 6 - DNA, 762 bp, Homo sapiens
  source 1..762: mol_type = genomic DNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 7 - DNA, 987 bp, Homo sapiens
  source 1..987: mol_type = genomic DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 8 - DNA, 462 bp, synthetic construct
  source 1..462: mol_type = other DNA
atgtaccggatgcagctgctgagctgtatcgccctgtctctggccctggtcacaaatagcgcccctaccagcagcagcaccaagaaaacacagctgcaactggaacacctcctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctga

SEQ ID NO: 9 - DNA, 762 bp, synthetic construct
  source 1..762: mol_type = other DNA
atgtggcctcctggatctgcttctcagcctcctccatctcctgccgctgctacaggacttcatcctgccgcaagacccgtgtctctgcagtgcagactgagcatgtgccccgccagatctctgctgctggtggctacactggtgctgctggatcatctgagcctggccagaaacctgccagtggccacgcctgatcctggcatgtttccttgtctgcaccacagccagaacctgctgagagccgtgtccaacatgctgcagaaggccagacagaccctcgagttctacccctgcaccagcgaggaaatcgaccacgaggacatcaccaaggacaagaccagcaccgtggaagcctgcctgcctctggaactgaccaagaacgagagctgcctgaacagcagagagacaagcttcatcaccaacggctcttgcctggcctccagaaagacctccttcatgatggccctgtgcctgagcagcatctacgaggacctgaagatgtaccaggtcgagttcaagaccatgaacgccaagctgctgatggaccccaagcggcagatcttcctggaccagaatatgctggccgtgatcgacgagctgatgcaggccctgaacttcaacagcgagacagtgccccagaagtccagcctggaagaacccgacttctacaagaccaagatcaagctgtgcatcctgctgcacgccttccggatcagagccgtgaccatcgacagagtgatgagctacctgaacgcctcctga

SEQ ID NO: 10 - DNA, 987 bp, synthetic construct
  source 1..987: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgtagctaa

SEQ ID NO: 11 - DNA, 1638 bp, synthetic construct
  source 1..1638: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggcagcggaggtggtggatctagaaatctgccagtggccacgcctgatcctggcatgtttccttgtctgcaccacagccagaacctgctgagagccgtgtccaacatgctgcagaaggccagacagaccctcgagttctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtcgagttcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctga

SEQ ID NO: 12 - DNA, 75 bp, synthetic construct
  source 1..75: mol_type = other DNA
agggccaagaggggcagcggcgagggcaggggcagcctgctgacctgcggcgacgtggaggagaaccccggcccc

SEQ ID NO: 13 - DNA, 66 bp, synthetic construct
  source 1..66: mol_type = other DNA
ggaagcggagctactaacttcagcctgctgaagcaggctggagacgtggaggagaaccctggacct

SEQ ID NO: 14 - DNA, 69 bp, synthetic construct
  source 1..69: mol_type = other DNA
ggaagcggacagtgtactaattatgctctcttgaaattggctggagatgttgagagcaaccctggacct

SEQ ID NO: 15 - DNA, 75 bp, synthetic construct
  source 1..75: mol_type = other DNA
ggaagcggagtgaaacagactttgaattttgaccttctcaagttggcgggagacgtggagtccaaccctggacct

SEQ ID NO: 16 - protein, 25 aa, synthetic construct
RAKRGSGEGRGSLLTCGDVEENPGP

SEQ ID NO: 17 - protein, 22 aa, synthetic construct
GSGATNFSLLKQAGDVEENPGP

SEQ ID NO: 18 - protein, 23 aa, synthetic construct
GSGQCTNYALLKLAGDVESNPGP

SEQ ID NO: 19 - protein, 25 aa, synthetic construct
GSGVKQTLNFDLLKLAGDVESNPGP

SEQ ID NO: 20 - protein, 15 aa, synthetic construct
GGGGSGGGGSGGGGS

SEQ ID NO: 21 - protein, 18 aa, synthetic construct
GGSSRSSSSGGGGSGGGG

SEQ ID NO: 22 - protein, 20 aa, synthetic construct
GGGGSGGGGSGGGGSGGGGS

SEQ ID NO: 23 - protein, 15 aa, synthetic construct
CGGGSGGGGSGGGGS

SEQ ID NO: 24 - protein, 15 aa, synthetic construct
SHGGHGGGGSGGGGS

SEQ ID NO: 25 - protein, 15 aa, synthetic construct
MGGMSGGGGSGGGGS

SEQ ID NO: 26 - protein, 15 aa, synthetic construct
YGGYSGGGGSGGGGS

SEQ ID NO: 27 - protein, 15 aa, synthetic construct
WGGYSGGGGSGGGGS

SEQ ID NO: 28 - protein, 12 aa, synthetic construct
SVSVGMKPSPRP

SEQ ID NO: 29 - protein, 12 aa, synthetic construct
VISNHAGSSRRL

SEQ ID NO: 30 - protein, 12 aa, synthetic construct
PWIPTPRPTFTG

SEQ ID NO: 31 - protein, 11 aa, synthetic construct
RGRGRGRGRGR